(19) United States
(12) Patent Application Publication
(10) Pub. No.: US 2017/0189444 A1
(43) Pub. Date: Jul. 6, 2017

(54) POLYMERIC COMPOSITIONS AND RELATED SYSTEMS AND METHODS FOR REGULATING BIOLOGICAL HYDROGELS

(71) Applicant: CALIFORNIA INSTITUTE OF TECHNOLOGY, Pasadena, CA (US)

(72) Inventors: Rustem F. ISMAGILOV, ALTADENA, CA (US); Sujit S. DATTA, ALHAMBRA, CA (US); Asher Preska STEINBERG, PASADENA, CA (US); Said R. BOGATYREV, PASADENA, CA (US)

(21) Appl. No.: 15/399,711

(22) Filed: Jan. 5, 2017

Related U.S. Application Data
(60) Provisional application No. 62/309,753, filed on Mar. 17, 2016.
(60) Provisional application No. 62/275,757, filed on Jan. 6, 2016.

(51) Int. Cl. A61K 31/765

(52) U.S. Cl. CPC A61K 31/765

(57) ABSTRACT

Polymeric composition and related methods and systems for regulating the structure of hydrogels are described. In particular, by varying the physiochemical properties of the polymeric composition, the structure of the hydrogels can be reversibly compressed or decompressed.

### CROSS-REFERENCE TO RELATED APPLICATION

The present application claims priority to U.S. Provisional Application No. 62/275,757, entitled “Polymer-Induced Colonic Mucous Hydrogel Compression” filed on Jan. 6, 2016, with docket number P1829-USP, and to U.S. Provisional Application No. 62/309,753, entitled “Polymeric Compositions and Related Systems and Methods for Regulating Biological Hydrogels” filed on Mar. 17, 2016, with docket number P1829-USP2, the contents of each of which is incorporated herein by reference in its entirety.

### STATEMENT OF GOVERNMENT GRANT

The invention was made with Government support in part by DARPA Biological Robustness in Complex Settings (BRICS) contract HR0011-15-C-0093, the National Science Foundation's Emerging Frontiers in Research and Innovation Award under Grant No. 1137089 and NSF Graduate Research Fellowship DGE-1144469 (to A.P.S.).

### FIELD

The present disclosure relates to polymeric compositions and, in particular, polymers capable of regulating hydrogel structures and related systems and methods.

### BACKGROUND

Hydrogels are integral components of biological systems. Despite of the effort and advancement in this field, how the structures of hydrogels can be influenced and controlled remains unclear. Challenges remain in the development of materials with desired physicochemical properties that can regulate biological hydrogel structures with applications in various technical fields including biomedical, medical diagnostics and therapeutics.

### SUMMARY

Provided herein, are polymeric compositions, and related systems and methods for regulating the structure of biological hydrogels. In several embodiments, the polymeric compositions are capable of compressing and/or decompressing the biological hydrogel by changing the overall volume, thickness and/or mesh size of the biological hydrogel.

According to a first aspect, a method to control an overall volume of a biological hydrogel is described. The method comprises contacting the biological hydrogel with one or more polymers of a molecular weight from 100 Da to 5 MDa at a concentration from 0.05-80% w/v, the molecular weight and concentration selected to obtain a change in the overall volume of the biological hydrogel according to a Flory-Huggins model.

According to a second aspect, a method to control an overall volume, mesh size and/or thickness of a biological hydrogel having an elastic modulus is described. The method comprises contacting the biological hydrogel with one or more polymers having a molecular weight from 100 Da to 5 MDa at a concentration from 0.05-80% w/v. In the method, the molecular weight and the concentration are selected to modify an osmotic pressure difference between an external osmotic pressure externally applied to an external surface of the biological hydrogel and an internal osmotic pressure internally applied to the external surface of the biological hydrogel. An increased osmotic pressure difference results in a more compressed state while a decreased osmotic pressure difference results in a less compressed, i.e. more decompressed state.

According to a third aspect, a method to compress a biological hydrogel is described. In the method the biological hydrogel has an elastic modulus, a basal surface in contact with an epithelial cell and an external surface opposite to the basal surface, the external surface in contact with an external environment. In the biological hydrogel at a base state, an osmotic pressure difference between an external osmotic pressure externally applied to the external surface of the biological hydrogel and an internal osmotic pressure internally applied to the external surface of the biological hydrogel is less than 10% of the elastic modulus of the biological hydrogel.

The method comprises contacting the biological hydrogel with one or more polymers to provide, after the contacting, an external polymeric osmotic pressure externally applied to the external surface of the biological hydrogel and an internal polymeric osmotic pressure internally applied to the external surface of the biological hydrogel, with an osmotic pressure difference between the external polymeric osmotic pressure and the internal polymeric osmotic pressure, following the contacting, equal to or higher than about 10% of the elastic modulus of the biological hydrogel.

According to a fourth aspect, a method to compress a mucus layer under a physiological osmotic pressure is described. The method comprises contacting the colonic mucus hydrogel with one or more polymers selected to provide an osmotic pressure difference equal to or greater than 10% of the elastic modulus of the colonic mucus and a total osmotic pressure lower than the physiological osmotic pressure (0.74 MPa).

According to a fifth aspect, a polymeric composition to control a structure of a biological hydrogel is described. The polymeric composition comprises in a suitable vehicle, one or more polymers of a molecular weight from 100 Da to 5 MDa at a concentration from 0.05-80% w/v, the molecular weight and the concentration of the one or more polymers being selected to obtain a change in the overall volume, mesh size and/or thickness of the biological hydrogel according to methods herein described.

According to a sixth aspect, a system to control a structure of a biological hydrogel is described. The system comprises one or more polymeric compositions herein described and a look-up table connecting one or more molecular weight and/or one or more concentrations of one or more polymers in the one or more polymeric compositions with at least one of the one or more overall volumes, one or more mesh size and one or more thicknesses of the biological hydrogel, the connecting performed according to a method of the present disclosure.

According to a seventh aspect, a system to control a structure of a biological hydrogel is described. The system comprises one or more polymeric compositions herein described and a look-up table connecting one or more molecular weight and/or one or more concentrations of one or more polymers in the one or more polymeric compositions with a percentage of compression and/or a percentage of decompression of the biological hydrogel according to methods herein described.

Methods herein described and related systems and polymeric compositions are known or expected in several embodiments to affect the structure of biological hydrogels such as colonic mucus, airway mucus, nasal mucus, cervico-vaginal mucus, extracellular matrix in tissues or biofilms.

Methods and systems herein described and related systems and polymeric compositions, are known or expected in several embodiments to affect metabolism of dietary and/or therapeutic compounds, or the related processing by microbes in the gastrointestinal tract of an individual

Methods and systems herein described and related systems and polymeric compositions are known or expected in several embodiments to alter access of pathogens and/or toxins to the epithelium.

Methods herein described and related systems and polymeric compositions are known or expected in several embodiments to design polymer-based therapeutics to controllably and predictably alter the morphology of mucus.

Methods herein described and related systems and polymeric compositions herein described can be used in connection with various applications wherein control of the structure of a biological hydrogel is desired. For example, methods herein described and related polymeric compositions herein described can be used in several fields including basic biology research, applied biology, bio-engineering, medical research, medical diagnostics, therapeutics, in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure. Exemplary embodiments of the present disclosure will become more fully understood from the detailed description and the accompanying drawings, wherein:

FIG. 1 shows in one embodiment polymers compress colonic mucus hydrogel in vivo. Panel A: Schematic depicting visualization of adherent colonic mucus hydrogel. Panel B: Side view confocal micrograph showing FC oil-mucus interface (top of figure, in gray) separated from the epithelial surface (bottom of figure, in gray) by the adherent mucus hydrogel (depicted in black). Scale bars, 30 μm. Panel C: Schematic of side view shown in Panel B. Panel D: FC oil mucus thickness measurements for colonic explants taken from SPF mice fed ad libitum on either a standard chow diet, 5% w/v sucrose in 1× PBS, or 5% w/v sucrose with 7% w/v PEG 200 k in 1× PBS. Data show means ±SEM.

FIG. 2 shows in one embodiment polymers compress colonic mucus hydrogel ex vivo. Panel A: Bright-field (top), confocal reflectance (middle), and two-photon (bottom) micrographs of epithelial surface. Image levels were adjusted for clarity. Scale bars, 30 μm. (b, c, e) Left shows schematics, right shows side view confocal micrographs. Scale bars, 10 μm. Panel B: Penetration of mucus by low concentration (0.05% w/v) of mPEG-FITC 200k. Panel C: Exclusion from mucus of 1 μm microparticle probes. Panel D: Schematic depicts mucus mesh structure, with penetrating probes on the left and larger non-penetrating probe on the right. Panel E: Top shows probe size distributions measured using dynamic light scattering (left axis, arrows to the left) or optical microscopy (right axis, arrows to the right). Bottom panel shows minimal probe separation from epithelial surface. Horizontal positions and error bars show geometric mean±geometric SD of lognormal fits to size distributions. Vertical positions and error bars show mean±SD. Grey bar shows mean of FC oil measurements of in vivo thickness for mice fed chow. Penetration measurements used fluorescently labeled polymers at concentrations below those that cause mucus compression. Panel F: Compression of colonic mucus by 3.5% w/v PEG 200 k.

FIG. 3 shows in one embodiment that tunable compression of colonic mucus hydrogel can be qualitatively described by Flory-Huggins theory. Panel A: Theoretically-predicted and Panel B: experimentally-measured (using 1 μm microparticles) mucus compression for varying polymer concentrations and molecular weights. Bold curves in Panel A show model results for parameter values χ_{SM}=0 and χ_{MP}=0.3; less opaque and dashed curves show sensitivity to variations in these parameters (upper and lower less opaque curves, χ_{SM}=0.1 and −0.1; upper and lower dashed curves, χ_{MP}=0.2 and 0.4). All curves below the text “PEG 200 k” indicate model results with the molecular weight of polyethylene glycol equal to 200 kDa. The curves above the text “PEG 200 k” and below the text “PEG 6k” indicate model results with the molecular weight of polyethylene glycol equal to 6 kDa. The curves above the text “PEG 6 k” and below the text “PEG 400” indicate model results with the molecular weight of polyethylene glycol equal to 400 Da. All mice, except for those indicated by upward triangles, were male. Symbols in Panel B indicate different mouse types and experimental conditions: squares, C57BL/6 mice; circles, BALB/c mice; upward triangles, female C57BL/6 mice; vertical diamond, washed explants from GF mice; downward triangles, all solutions have added 2× Roche protease inhibitor cocktail; pentagons, all solutions have added 5 mM MgSO₄; horizontal diamonds, experiments performed at 37° C. instead of 22° C. using a heated microscope stage; stars, polyacrylic acid of ˜8 kDa average molecular weight instead of PEG; hexagons, HEPES buffer instead of PBS for all solutions. Markers with crosses through them or a single dot in the center indicate results of experiments performed with PEG 400 Da. Markers that are solid in color indicate results of experiments performed with PEG 6 kDa. Open face markers indicate results of experiments performed with PEG 200 kDa. Each data point represents the mean of a series of five measurements on a single explant; error bars represent measurement uncertainty. Panel C: Schematic showing one effect potentially underlying mucus compression: molecular weight-dependent partitioning of the polymer.

FIG. 4 shows in one embodiment that gut microbes can modulate mucus compression by modifying the polymeric composition of intestinal contents. Panel A: Mucus compression induced by dietary polymers, determined using the ex vivo microparticle method. Each data point represents the mean of a series of five measurements on a single explant; error bars represent measurement uncertainty. Markers with crosses through them indicate results of experiments performed with dextrin. Open face markers indicate results of experiments performed with pectin. Solid markers indicate results of experiments performed with pullulan. Inset shows data for pectin and pullulan with semilogarithmic axes. (b and c) Mucus (Panel B) thickness or (Panel C) compression measurements determined using (dark gray) ex vivo microparticle method or (light gray) FC oil method, for explants from SPF or GF mice. Last bar in (b) shows measurements for washed GF explants. Data are presented as means±SEM. Panel D: Schematic depicting how microbial degradation of polymers alters mucus compression.

FIG. 5 shows in one embodiment images of murine epithelium in the xy and xz planes. (Panel A) Two-photon and (Panel B) bright-field micrographs of unwashed epithelium from a mouse fed standard chow, imaged under FC oil. (c, d) Side views of lectin-stained epithelium washed with saline and imaged under aqueous solutions. Staining was performed by incubating a colon explant with 200 μL of a test solution of 2 mg/mL Rhodamine Ulex Europaeus Agglutinin I (Vector Laboratories, Burlingame, Calif., USA), which stains a-L-fucose residues on the surface of epithelial cells, in HEPES buffer in a sealed petri dish for 10 min at 4° C., then washing the exposed luminal side with several milliliters of ice-cold 1× PBS. The explant surface was then immediately imaged using (Panel C) confocal fluorescence microscopy (543 nm excitation/560 nm long-pass filter) and (Panel D) confocal reflectance microscopy (514 nm excitation/505 nm long-pass filter). Epithelial surface is indicated by arrows to the right of the figure, confirming that the position of the epithelium agrees between the different imaging modalities. The adherent mucus hydrogel overlies the epithelium in the direction of increasing z above the green arrows. All scale bars, 30 μm.

FIG. 6 illustrates in one embodiment false-color side view showing WGA-stained adherent mucus hydrogel. 1 μm diameter microparticles was first deposited onto the explant surface of a freshly excised, washed, and mounted colonic explant. After incubating for 1 h at 4° C., the colonic mucus was then stained with wheat germ agglutinin (WGA), a fluorescent lectin that specifically binds to sialic acid sugar residues in the mucins. 10 μg/mL of WGA-Oregon Green (Invitrogen, Grand Island, N.Y., USA) was prepared in 1× PBS, a ˜0.5 mL drop was placed on the exposed surface of the explant and the sealed petri dish was incubated for 5 min at room temperature. The exposed surface was then washed with several milliliters of ice-cold 1× PBS and the explant surface (lower surface labeled to the right of the figure with the text “Epithelium”) and the deposited 1 μm microparticles (upper circles that appear in white or light gray and are labeled with arrows and to the right of figure with the text “Particles”) were immediately imaged using confocal reflectance microscopy, and the stained mucus hydrogel imaged using confocal fluorescence microscopy (488 nm excitation/505 nm long-pass filter). The mucus hydrogel is indicated by the label “Adherent Mucus Hydrogel” to the right of the figure, and is the region between the white particles and the white regions that appear between the epithelium and hydrogel region. Image is a superimposition of two separate, parallel side views taken at two neighboring positions in the xy plane. It was observed that the position of the deposited microparticles agrees with the top of the stained mucus hydrogel. Scale bars, 30 μm.

FIG. 7 shows in one embodiment co-localization of signal from microparticle probes and epithelium from different imaging modalities. (Panel A) Brightfield, (Panel B) fluorescence excitation and (Panel C) reflectance images of 1 μm probes of the same xy slice. (Panel D) An xz side view of fluorescence signal from 1 μm probes. (Panel E) The same xz side view as in panel d but of the reflectance signal from 1 μm probes and epithelial surface. (Panel F) Brightfield and (Panel G) reflectance images of the epithelial surface of the same xy slice. The arrow linking panel (c) to panel (e) indicates the vertical position of the xy slice shown in panels (a)-(c). The arrow linking panel (g) to panel (e) indicates the vertical position of the xy slice shown in panels (f)-(g). Scale bars, 30 μm. This confirms that the positions of the microparticles given by confocal reflectance and confocal fluorescence microscopy agree.

FIG. 8 shows in one embodiment overview of image processing of confocal side views. To eliminate artifacts associated with staining and accelerate image acquisition, label-free confocal reflectance microscopy was used to simultaneously image the underlying epithelium (lower surface) and the microparticles deposited on the adherent mucus hydrogel (upper bright spots). To obtain the false-color side views, each side view was first thresholded; (Panel A) shows a representative xz side view before processing, while (Panel B) shows image after thresholding, with uniform enhancement of brightness and contrast across the entire image. The image was then split into two parts, and the epithelium was false-colorized green (shown in gray in the illustration of the panel) (Panel C) and the deposited microparticles or oil-mucus interface (for imaging of unwashed tissues with FC oil) were false-colorized magenta (shown in gray in the illustration of the panel) (Panel D). Dashed lines indicate where images (c)-(d) were split. Merging these two channels produced the side view images shown, exemplified by (Panel E). Scale bars, 30 μm. Unless otherwise noted, all of the experiments mapped z ranges spanning from below the epithelial surface to well above the mucus hydrogel surface. Each of the side view images presented in this paper was cropped and scaled in xz for clarity (indicated by the x and z scale bars), to focus on the region corresponding to the mucus hydrogel.

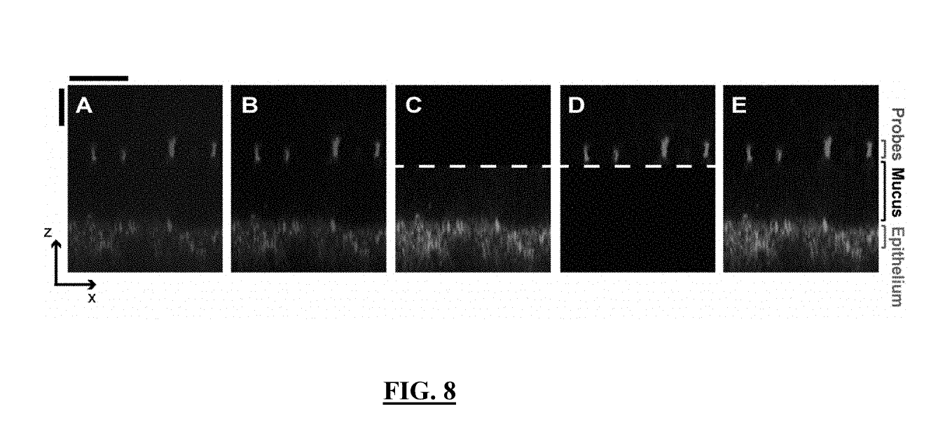

FIG. 9 demonstrates in one embodiment false-color side views (xz plane) of 3D stacks showing probes excluded from (top row) or penetrating (bottom row) the mucus hydrogel. (Panel A) Mixture of both 250 nm and 1 μm microparticles and (Panel B) 500 nm particles were excluded from the adherent mucus hydrogel. The probes (shown in gray, labeled on the right side of each image as “250 nm+1 micron Probes” and “500 nm Probes”, respectively) were unable to diffuse through the mucus, and instead deposited on top of the hydrogel. The probes and the epithelium were simultaneously imaged using (Panel A) 514 nm excitation/505 nm long-pass filter and (Panel B) 800 nm excitation/650 nm long-pass filter. (Panel C) Fluorescent PEG 200 kDa, (Panel D) fluorescent dextran 2 MDa, (Panel E) fluorescent 100 nm microparticle probes all penetrate the hydrogel. Note that for Panel C and D that the top of the epithelium is the white region that appears in the image and is further denoted by the text “Epithelium” on the right side of each image. Note that for Panel E, the epithelium is the dark region at the bottom of the image, that is further denoted by the text “Epithelium” on the right side of the image. Note that polymers in (Panel A) and (Panel B) were used at concentrations below those that cause mucus compression. The probes (shown in gray) diffused through the mucus and reached the underlying epithelium (shown in white), except for some isolated regions immediately adjacent to the epithelium observed in some experiments (dark patches). The probes were imaged using confocal fluorescence microscopy (488 nm excitation/505 nm long-pass filter) and the epithelium was imaged using confocal reflectance microscopy. The adherent mucus hydrogel overlies the epithelium in the direction of increasing z solid and dashed white lines in panel (Panel C) indicate the approximate average and maximal positions of the top of the mucus, measured using 1 μm microparticles. Scale bars, 30 μm. In each experiment using probes of different sizes, after placing the test solution onto the exposed luminal surface, the tissue was incubated at 4° C. for 1-2 h before imaging the explant. It was estimated that the time required for probes 100 nm or smaller to diffuse through the mucus is <10 min, and the time required for the 250 nm probes to diffuse across the vertical extent of the mucus in free solution as being ˜10 min, both much shorter than the incubation time. It was thus deduced that the fluorescent probes smaller than the measured mucus mesh size had sufficient time to diffuse through the mucus to the underlying epithelium, and that the measured exclusion of the larger probes reflects the presence of the adherent mucus hydrogel.

FIG. 10 demonstrates in one embodiment side view showing penetration of mucus hydrogel by polymers. The polymer self-diffusion coefficient in the free solution outside the mucus, D_{free}, is represented by D₀ for the dilute polymer solutions, and can be estimated as D_{free}≈D₀(c/c*)^{-7/4} for the polymer solutions that were above their overlap concentration c*. Our experiments spanned D₀≈10⁻¹¹-3×10⁻¹⁰ m²/s and c/c*≈0-10, therefore D_{free}≈2×10⁻¹³-3×10⁻¹⁰ m²/s. The characteristic time taken for the polymers to diffuse through the mucus can thus be estimated as ranging from ˜1 s to 1 h, shorter than the time taken to perform the experiments. It was thus assumed that the polymer molecules were able to diffuse through the mucus hydrogel before imaging commenced in all of the experiments. To study the steady-state penetration of the PEG into the adherent mucus hydrogel, two representative test solutions were imaged: (Panel A) 13% w/v PEG 6 k spiked with 0.5 mg/mL FITC-PEG 5 k, and (Panel B) 3% w/v PEG 200 k spiked with 0.6 mg/mL FITC-PEG 200 k. Consistent with the expectation, in both cases, the polymer penetrated through the adherent mucus hydrogel and reached the underlying epithelium. Traces show the spatial variation of the x-averaged probes fluorescence intensity for the region indicated by the dashed black box. The probes (gray) diffused through the mucus and reached the underlying epithelium (white or light gray). The probes were imaged using confocal fluorescence microscopy and the epithelium was imaged using confocal reflectance microscopy. The adherent mucus hydrogel overlies the epithelium in the direction of increasing z above the epithelium; solid and dashed white lines show the average and maximal positions of the top of the mucus, measured using 1 μm microparticles. Scale bars, 30 μm.

FIG. 11 shows in one embodiment fluorescence profiles of test solutions deposited on mucus hydrogel, before and after washing. It was expected that the carboxyl groups on the mucin sialic acid residues were negatively charged in our experiments (pH˜7), and therefore, complexation between the added PEG and the mucins is minimal. Moreover, PEG solutions are not exposed to light while being kept at low temperatures when not in use, to minimize oxidation. To confirm that labeled PEG molecules were not chemically cross-linked to the mucus hydrogel as they diffused through the hydrogel, four sets of fluorescence measurements were performed, using as test solutions (Panel A) 50 μM fluorescein, (Panel B) 15 μM FITC-PEG 350, (Panel C) 6 μM FITC-PEG 5 k, (Panel D) 15μM FITC-PEG 350 in 60% w/v PEG 400. Four different explants were incubated with 1 μm microparticles for >1 h, then imaged using confocal reflectance (to identify epithelial surface and microparticles on mucus) and confocal fluorescence (to quantify fluorescence of deposited test solution). Curves show fluorescence profiles of test solutions: horizontal axis shows measured fluorescence, averaged over a 450 μm×450 μm xy field of view, while vertical axis shows z position. Green and magenta arrows show average positions of epithelial surface and probes deposited on the mucus hydrogel surface. PBS was first used as the test solution to provide a measure of background fluorescence (indicated with the black line and text “Before”). Dyed test solution was then deposited on the mucus (indicated with the black line and text “Test”). The explant was then washed with saline (indicated with the black line and text “Wash”). Fluorescence profiles returned to background levels after washing, suggesting that strong chemical interactions (such as covalent reactions) between the labeled PEG and the mucus hydrogel do not occur. The same gain settings were used before and after.

FIG. 12 demonstrates in one embodiment that optical properties of polymer solutions do not appreciably affect z measurements. (Panel A) Schematic showing set up of control experiments, measuring separation between two parallel glass plates using the same confocal reflectance microscopy approach. The test solution infiltrated the open gap between the two plates. (Panel B) Separation was first quantified using PBS as the test solution filling the space between the two plates, and then either 10% PEG 200 k (test case 1), or 60% PEG 400 (test case 2) was used as the test solution. Introduction of the polymer solution did not change the measured z separation appreciably, indicating that optical effects due to the presence of the polymer solution did not significantly affect the z measurements.

FIG. 13 shows in one embodiment the sensitivity of model predictions to variations in numerical parameters. Each panel shows numerical calculations of the mucus hydrogel compression for different concentrations of PEG 400, PEG 6 k,and PEG 200 k.The molecular weight of the polymer of each curve(400, 6 k or 200 k) is indicated with arrows. Note that due to the constraint derived in the initial polymer-free case, some of the parameters are coupled and cannot be varied independently. (Panel A) v⁰_{M} values are varied and corresponding values of N_{M} are adjusted to satisfy the initial polymer-free constraint. Light, solid traces correspond to v⁰_{M}=0.07 and N_{M}=628, and light, dashed traces correspond to vhu 0_{M}=0.35 and N_{M}=2026. Note the overlap between the solid and dashed traces. (Panel B) χ_{SM} values are varied and corresponding values of N_{M} are adjusted to satisfy the initial polymer-free constraint. Light, solid traces correspond to χ_{SM}=−0.2 and N_{M}=715, and light, dashed traces correspond to χ_{SM}=0.45 and N_{M}=9425. Upper and lower less opaque curves in FIG. 13 (Panel B) which correspond to χ_{SM}=0.1 and −0.1, were characterized by N_{M}=1247 and N_{M}=833. (Panel C) The number of Kuhn segments y for each PEG molecule is varied. Light, solid traces correspond to y=1, 2, and 76, and light, dashed traces correspond to y=1, 11 and 611 for PEG 400, 6 k, and 200 k respectively. (Panel D) χ_{MP} is varied. Light, solid traces correspond to χ_{MP}=0 and light, dashed traces correspond to χ_{MP}=0.5. In each panel, the dark solid traces are the simulations presented in FIG. 13 (Panel D). In all cases, similar trends of compression was observed with polymer concentration and molecular weight as in the experiments. (Panel E) Numerical calculations showing the partitioning between the hydrogel and solution phase for PEG 400 (orange), 6 k (blue), and 200 k (green). The ratio of PEG inside and outside the hydrogel (v_{P}ⁱⁿ/φ, denoted “Partitioning”) is plotted against the PEG concentration outside the hydrogel. Consistent with our expectation, the higher molecular weight polymer is more likely to be excluded from the mucus hydrogel.

FIG. 14 shows in one embodiment gel permeation chromatography of luminal contents from SPF and GF mice. An Agilent 1100 HPLC with a binary pump and auto-sampler as used, which was connected to a Tosoh TSKgel G3000SWx1 column equilibrated with 1× PBS, pH 7.4, flow rate: 0.7 ml/min. For detection of the polymers, a Wyatt DAWN HELEOS light scattering instrument with a Wyatt Optilab Rex refractive index detector was used. Detected peaks were analyzed using ASTRA V software. For the pullulan standards, the Agilent PL 2090-0101 Pullulan polysaccharide calibration kit (Agilent, Wilmington, Del., USA) was used. An injection volume of 50 μL was used for each. All samples were prepared in lx phosphate buffered saline and run through a sterile syringe filter (Polyvinylidene Fluoride, 13 mm diameter, pore size of 0.22 μm, Fisherbrand, Pittsburgh, Pa., USA) before injection. For luminal contents, on the day of the experiment, frozen liquid fractions were warmed to room temperature for 10-20 min, then diluted two-fold with 1× PBS. Samples were centrifuged at 12,000 g at 4° C. for 2 h in sterile centrifugal filters (Polyvinylidene Fluoride, pore size 0.22 μm, from EMD Millipore, Billerica, Mass., USA). After centrifugation, samples were allowed to equilibrate to room temperature for 30 min before injection. For all liquid fraction samples, an injection volume of 10 μL was used. If multiple runs were performed on the same sample, the remaining sample volume was stored at 4° C. until prior runs were complete. (Panel A) Chromatograms of luminal contents from four, 3-month-old SPF males (labeled “SPF”) and two male and one female, 4-month-old GF (labeled “GF”) mice. Differential refractive index (dRI) is plotted against time (min). Both runs were run on the same day. (Panel B) Chromatograms of luminal contents of GF mice (labeled “GF” with arrow) and pullulan standards (labeled i-vii). Differential refractive index (dRI) is plotted against time (min). Concentrations and peak average MWs of the standards used were: (i) 5 mg/ml 180 Da, (ii) 8 mg/ml 667 Da, (iii) 4 mg/ml 6,100 Da, (iv) 4 mg/ml 9,600 Da, (v) 1 mg/ml 47,100 Da, (vi) 1 mg/ml 107,000 Da, (vii) 1 mg/ml 194,000 Da, 344,000 Da and 708,000 Da.

FIG. 15 shows published measurements of the osmotic pressure of PEG 400, PEG 6 k, and PEG 200 k solutions.

FIG. 16 shows experimental data for PEG 400, PEG 6000, and PEG 200 k, with the % compression as a function of the total osmotic pressure of each polymer solution. Solid markers are for PEG 200 k, markers with diagonal crosses are for PEG 6 k and open face markers are for PEG 400.

### DETAILED DESCRIPTION

Provided herein are polymeric compositions and related methods and systems to control the structure of biological hydrogels.

The term “polymeric compositions” as used herein refers to a composition comprising one or more polymeric molecules of molecular weight of at least 100 Da and a suitable vehicle. In polymeric compositions herein described, each of the one or more polymeric molecules comprises repeating structural units connected one with another to form chain of various lengths with or without branches. An oligomer as used herein is defined as a polymer containing a total of less 25 or less monomeric moieties.

In some embodiments herein described, polymers comprised in the polymeric compositions of the disclosure are hydrophilic or amphiphilic possessing both hydrophilic and lipophilic properties. In particular, in a polymer of the polymeric compositions herein described at least a portion is substantially water soluble, where the term “substantially water soluble” as used herein with reference to a polymer indicates the ability of the polymer to dissolve in water such that the amount of solubilized polymer has a target concentration as specified above (e.g. 0.05-80%) Appropriate amphiphilic or hydrophilic polymers can be identified by a skilled person in view of the present disclosure. For example solubility of the polymer can be identified in view of solubility parameters (δ) of the polymer backbone, as well as by determining the Flory-Huggins interaction parameter (χ) from the solubility parameters according to calculations described herein. In particular, an exemplary reference providing solubility parameters is the website www.sigmaaldrich.com/etc/medialib/docs/Aldrich/General_Information/polymer_solutions.Par.0 001.File.tmp/polymer_solutions.pdf at the time of filing of the present. More particularly, a skilled person will know that Sigma-Aldrich and other chemical companies provide exemplary tables showing exemplary solubility paramenter values for various compositions and polymers. A skilled person can also refer to sources such as the Polymer Handbook to find solubility parameter values (115). Substantially soluble water polymers comprise polar, positively charged and/or negatively charged polymers.

In embodiments of the present disclosures, polymers comprised in a polymeric composition include at least two monomeric moieties presenting a number n of functional groups, FG conferring hydrophilicity and charge character to the polymer.

The at least two monomeric moieties are selected to be biologically acceptable and therefore be compatible with the biological hydrogel to be controlled and the environment where the biological hydrogel to be controlled is located.

The term “functional group” as used herein indicates specific groups of atoms within a molecular structure that are responsible for the characteristic physical and/or chemical reactions of that structure and in particular to physical and/or chemical associative interactions of that structure. Exemplary functional groups that can provide hydrophilicity and charge character to a polymer comprise amino group provide positive charge and carboxyl group which provide negative charge character to the polymer under appropriate pH conditions, while hydroxyl group and glycol ether provide hydrophilicity.

In polymers herein described some functional groups presented on the polymer can be capable to bind to corresponding functional groups presented on another polymer or other molecule. As used herein, the term “corresponding functional group” or “complementary functional group” refers to a functional group that can react, and in particular physically or chemically associate, to another functional group. Thus, functional groups that can react, and in particular physically or chemically associate, with each other can be referred to as corresponding functional groups. Some of exemplary corresponding functional groups comprise for example, carboxylic acids with other carboxylic acids, carboxylic acids with amines, alcohols with amines, alcohols with carboxylic acids, diacetamidopyridine with thymine, the Hamilton Receptor with cyanuric acid, and others identifiable to a skilled person

In embodiments herein described, a polymer that can be provided in compositions methods and system of the disclosure can have formula I

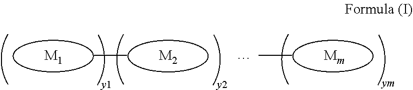

wherein M1 to Mm are monomers formed by a same or different biologically acceptable organic moiety capable to bind one another to form a polymer, wherein y1, y2 to ym are independently ≧0 and wherein at least one of the monomer M1 to Mm present one or more functional groups FG1 to FGn conferring hydrophilicity to the polymer.

In some embodiments, the polymers can have linear, branched, hyperbranched, bottlebrush structures or combinations thereof. In particular, bottlebrush polymers are type of branched or graft polymer with polymeric side-chains attached to a linear backbone. Bottlebrush polymer can have large size, exceeding lengths of 100 nm in some embodiments. Due to their large size and densely crowded side-chains, bottlebrush polymers provide unique characteristics including high entanglement molecular weight, ability to rapidly self-assemble of bottlebrush block copolymers into large domain structure, functionalization of bottlebrush side-chains for recognition, imaging or drug delivery in aqueous environments, and others as will be understood by a person skilled in the art.

Exemplary biologically acceptable organic moieties forming monomers in polymers herein described comprise natural or unnatural amino acids, nucleotides, monosaccharides, and ethylenic monomers.

As used herein the term “amino acid”, “amino acid monomer”, or “amino acid residue” refers to any of the naturally occurring amino acids, any non-naturally occurring amino acids, and any artificial amino acids, including both D and L optical isomers of all amino acid subsets. In particular, amino acid refers to organic compounds composed of amine (—NH2) and carboxylic acid (—COOH), and a side-chain specific to each amino acid connected to an alpha carbon. Different amino acids have different side chains and have distinctive characteristics, such as charge, polarity, aromaticity, reduction potential, hydrophobicity, and pKa. Amino acids can be covalently linked to forma polymer through peptide bonds by reactions between the amine group of a first amino acid and the carboxylic acid group of a second amino acid. Exemplary amino acids comprise any of the twenty naturally occurring amino acids, non-natural amino acids, and artificial amino acids and include both D and L optical isomers. In particular, non-natural amino acids include D- stereoisomers of naturally occurring amino acids (these including useful ligand building blocks because they are not susceptible to enzymatic degradation).

The term “unnatural amino acid” refers to a synthetic amino acid not normally found in a biological system. Examples of “unnatural amino acid ” include but are not limited to norleucine, norvaline, alloisoleucine, allothreonine, homoarginine, thioproline, dehydroproline, hydroxyproline, pipecolic acid, azetidine acid, homoserine, cyclohexylglycine, alpha-amino-n-butyric acid, cyclohexylalanine, aminophenylbutyric acid, phenylalanine mono and di-substituted at the positions ortho, meta and para of the aromatic ring, O-alkylated derivatives of serine, threonine and tyrosine, S-alkylated cysteine, epsilon-alkylated lysine, delta-alkylated ornithine, aromatic amino acids, substituted at the positions meta or para of the ring such as phenylalanine-nitrate, -sulfate, -phosphate, -acetate, -carbonate, -methylsulfonate,—methylphosphonate, tyrosine-sulfate, -phosphate, -sulfonate, -phosphonate, para-amido-phenylalanine, C-alpha,alpha-dialkylated, amino acids such as alpha,alpha-dimethylglycine (Aib), alpha-aminocyclopropanecarboxylic acid (Ac3c), alpha-aminocyclobutane-carboxylic acid (Ac4c), alphaminocyclopentanecarboxylic acid (Ac5c), alpha-aminocyclohexanecarboxylic acid (Ac6c), diethylglycine (Deg), dipropylglycine (Dpg), diphenylglycine (Dph). Examples of beta-amino acids are beta-alanine (beta-Ala), cis and trans 2,3-diaminopropionic acid (Dap).

The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Exemplary functional groups that can be comprised in an analog include methyl groups and hydroxyl groups and additional groups identifiable by a skilled person. Exemplary monomers of a polynucleotide comprise deoxyribonucleotide, ribonucleotides, LNA nucleotides and PNA nucleotides. The term “deoxyribonucleotide” refers to the monomer, or single unit, of DNA, or deoxyribonucleic acid. Each deoxyribonucleotide comprises three parts: a nitrogenous base, a deoxyribose sugar, and one or more phosphate groups. The nitrogenous base is typically bonded to the 1′ carbon of the deoxyribose, which is distinguished from ribose by the presence of a proton on the 2′ carbon rather than an -OH group. The phosphate group is typically bound to the 5′ carbon of the sugar. The term “ribonucleotide” refers to the monomer, or single unit, of RNA, or ribonucleic acid. Ribonucleotides have one, two, or three phosphate groups attached to the ribose sugar

The term “monoscaccharide” refers to a carbohydrate unit that is not decomposable into simpler carbohydrate units by hydrolysis, is classed as either an aldose or ketose, and contains one or more hydroxyl groups per molecule. Exemplary monosaccharides comprise glucose, fructose, or ribose. Additionally, exemplary amino monosaccharide includes but is not limited to N-acetylglucosamine, sialic acids such as neuraminic acid, D-galactosamine. Monosacchardides can occur naturally or be chemically synthesized. Monosaccharide monomers can be bound one to another by glycosidic bond which can be an alpha or a beta glycosidic bond. For example, cellobiose, Formula (IIa), consists of two glucose moieties linked by a beta (1→4) glycosidic bond.

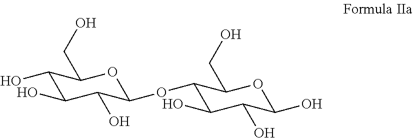 [SMILES: OCC1OC(OC2C(CO)OC(O)C(O)C2O)C(O)C(O)C1O]

In another example, alpha-maltose, Formula (IIb) consists of two glucose moieties linked by an alpha (1→4) glycosidic bond.

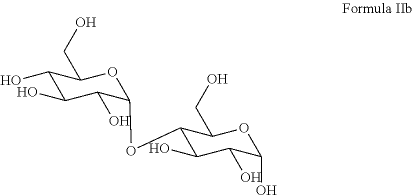 [SMILES: OCC1OC(OC2C(CO)OC(O)C(O)C2O)C(O)C(O)C1O]

In some embodiments, a polymer includes at least two monosaccharide moieties linked by at least one glycosidic bond. Exemplary neutral monosaccharide includes but is not limited to D-glucose, D-mannose, D-galactose, D-xylose, D-apiose, L-rhamnose, D-galactose, D-fructose, L-fucose, D-ribose, and L-arabinose. Exemplary carboxylic acid monosaccharide includes but is not limited to L-iduronic acid, 2-O-sulfo-L-iduronic acid (IdoA2S), D-glucopyranuronic acid, D-galacturonic acid.

An ethylenic monomer refers to an organic moiety formed by a substitute ethylene. Exemplary ethylenic monomers include vinyl pyrrolidone, alpha, beta-ethylenically unsaturated monocarboxylic acids such as acrylic acid, methacrylic acid, itaconic acid, and crotonic acid and their derivatives. Exemplary derivatives of alpha, beta-ethylenically unsaturated monocarboxylic acids include acrylamide, methacrylamide, alkyl acrylamides, dialkyl acrylamides, alkyl methacrylamides, dialkyl methacrylamides, alkyl acrylate, alkyl methacrylate.

An example of ethylenic monomers can be represented by Formula (IVa). R10 can be any one of FG1 to FGn or any group that presents FG1 to FGn of water soluble polymer P.

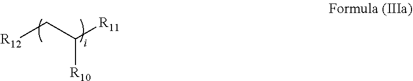

Wherein R₁₀ R₁₁ and R₁₂ i independently substituted or unsubstituted optionally heteroatom containing C1 to C10 alkly group.

The term “alkyl” as used herein refers to a linear, branched, or cyclic saturated hydrocarbon group typically although not necessarily containing 1 to about 10 carbon atoms, preferably 1 to about 6 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 6 carbon atoms. The term “cycloalkyl” intends a cyclic alkyl group, typically having 4 to 8, preferably 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl, respectively.

The term “heteroatom-containing” as in a “heteroatom-containing alky group” refers to a alkyl group in which one or more carbon atoms is replaced with an atom other than carbon, e.g., nitrogen, oxygen, sulfur, phosphorus, selenium or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. It should be noted that a “heterocyclic” group or compound may or may not be aromatic, and further that “heterocycles” can be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

In some embodiments, ethylenic monomers can be represented by Formula (IIIB).

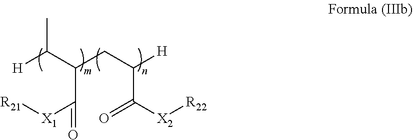

In which X1 and X2 can be independently selected from O or NH and R21 and R22 can be independently hydrogen or substituted or unsubstituted C1-C4 alkyl groups, m and n are independently selected from 1 to 100,000.

In some embodiments, ethylenic monomers can be represented by Formula (IIIc).

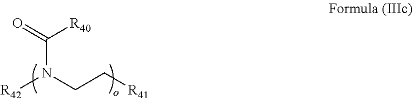

In Formula (IVd), each R₄₀, R₄₁ and R₄₂can be independently hydrogen or substituted or unsubstituted C1-C4 alkyl groups, o is selected from 1 to 100,000.

In various embodiments, one or more of the monomers herein described can present functional groups FG1 to FGn conferring the hydrophilicity and charge character to the polymer. For example, amino group and carboxyl group provide positive and negative charge character to the polymer under appropriate pH conditions, while hydroxyl group and glycol ether provide hydrophilicity.

In some embodiments, one or more monomers of a polymer herein described can be in free form with functional groups presented on the one or more monomers capable of performing coupling reactions and/or are otherwise reactive. In some embodiments, one or more monomers of a polymer herein described can be in a protected form in which the functional groups in the one or more monomers are not capable of performing coupling reactions and/or be otherwise reactive. A protected form can be converted to an unprotected form typically in a single chemical reaction step.

Exemplary polymers that can be used in compositions, methods and systems herein described are polypeptides, polynucleotides, polysaccharides, and synthetic polymers or semisynthetic polymers.

The term “polypeptide” as used herein indicates an organic linear, circular, or branched polymer composed of two or more amino acid monomers and/or analogs thereof. The term “polypeptide” includes amino acid polymers of any length including full length proteins and peptides, as well as analogs and fragments thereof. The term “amino acid analog” refers to an amino acid in which one or more individual atoms have been replaced, either with a different atom, isotope, or with a different functional group but is otherwise identical to original amino acid from which the analog is derived.

The term “polynucleotide” as used herein indicates an organic polymer composed of two or more monomers including nucleotides, nucleosides or analogs thereof. Accordingly, the term “polynucleotide” includes nucleic acids of any length, and in particular DNA, RNA, analogs thereof, such as LNA and PNA, and fragments thereof, possibly including non-nucleotidic or non-nucleosidic monomers, each of which can be isolated from natural sources, recombinantly produced, or artificially synthesized. Polynucleotides can typically be provided in single-stranded form or double-stranded form (herein also duplex form, or duplex).

The term “polysaccharide” as used herein refers to linear or branched polymeric carbohydrates composed of long chains of monosaccharide units bound together by glycosidic linkages. Polymeric carbohydrate molecules that can be comprised in polymeric composition herein described can be heteropolysaccharides in which more than one type of monosaccharide is present or homopolysaccharides in which all the monosaccharides in the polysaccharide are the same type. Oligosaccharides are carbohydrate polymers comprising three to ten monosaccharides. Polysaccharides comprise cellulose derivative, arabinoxylan, inulin, cereal β-glucans, alginic acid, guar gum, hydropropyl guar, Xanthan gum, chitosan, starch, dextrin, pectin, levan, elsinan, and pullulan. Modified starch as used here includes 1400 dextrin, 1401 acid-treated starch, 1402 alkaline-treated starch, 1403 bleached starch, 1404 oxidized starch, 1405 starches, enzyme-treated, 1410 monostarch phosphate, 1412 distarch phosphate, 1413 phosphated distarch phosphate, 1414 acetylated distarch phosphate, 1420 starch acetate, 1422 acetylated distarch adipate, 1440 hydroxypropyl starch, 1442 hydroxypropyl distarch phosphate, 1443 hydroxypropyl distarch glycerol, 1450 starch sodium octenyl succinate, 1451 acetylated oxidized starch, in which each four digit number, such as “1400” “1401”, refers to an E number, i.e. a code for a substance that is permitted to be used as a food additive for use within the European Union and Switzerland. Polymeric carbohydrate that can be used in compositions, methods and systems herein described can also comprise water soluble cellulose derivative such as methylcellulose, hydroxyethyl cellulose, hydroxypropyl cellulose (HPC), hydroxyethyl methyl cellulose, hydroxypropyl methyl cellulose (HPMC), carboxymethyl cellulose (CMC), sodium carboxymethyl cellulose, croscarmellose sodium.

Polysaccharides in the sense of the disclosure also comprise dietary fibers. The term “dietary fiber” refers to edible parts of plants or analogous carbohydrates that are resistant to digestion and absorption in the human small intestine with complete or partial fermentation in the large intestine. Analogous carbohydrates are synthesized dietary fibers that have demonstrated the physiological properties similar to those of naturally occurring dietary fibers. Exemplary dietary fiber includes polysaccharides, oligosaccharides, lignin, and associated plant substances.

Additional dietary fibers comprises polysaccharides, such as arabinoxylans, cellulose and other plant components such as resistant starch, resistant dextrins, inulin, lignin, chitins, pectins, beta-glucans and others that can be readily identified by a person skilled in the art.

Exemplary synthetic polymers include poly(N-vinylpyrrolidone), poly(acrylic acid), poly(methacrylic acid), poly(-hydroxyethyl methacrylate/methacrylic acid), Poly(ethylene/acrylic acid), poly(-hydroxypropyl methacrylate), poly(hydroxyethylmethacrylate), poly(-ethyl-2-oxazoline), polymethacrylamide, polyacrylamide, poly(N-iso-propylacrylamide), poly(-vinylpyridine), poly(-vinylpyridine N-oxide), poly(-vinylpyridine), poly(-vinylpyridine N-oxide), poly(-vinyl-1-methylpyridinium bromide), poly(ethylene oxide), poly(ethylene oxide-b-propylene oxide), poly(styrenesulfonic acid), sodium salt, poly(vinylsulfonic acid) sodium salt, poly(vinyl phosphoric acid), sodium salt, poly(vinyl alcohol), poly(allyl amine), poly(2-methacryloxyethyltrimethylammonium bromide), poly(N-vinylpyrrolidone/vinyl acetate), poly(butadiene/maleic acid).

In some embodiments, the polymer can be a polyethylene glycol (i.e. k=0), polypropylene glycol (i.e. j=0) or a polyethylene-b-polypropylene glycol (j>0, k>0) as illustrated by Formula (IVb).

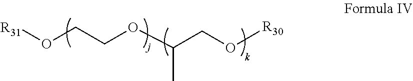

wherein the end groups R₃₀ and R₃₁ can be independently hydrogen or any substituted or unsubstituted C1-C6 alkyl or aromatic group.

In some embodiments, the polymers can be of animal origin, such as glycosaminoglycans, mucopolysaccharides, hyaluronan, chondroitin sulfate and others identifiable by a person skilled in the art. In some embodiments, the polymeric composition can contain polymers such as soluble mucins that are not crosslinked to form a mucus hydrogel network.

In embodiments herein described polymers have a molecular weight at least 100 Da, preferably in a range between 100 Da and 5 MDa, more preferably in a range between 200 kDa and 5 MDa.

In some embodiments, the polymeric composition used herein has a hydrodynamic radius in a range between 1 nm and 1000 nm. The term “hydrodynamic radius” used herein describes the size of a polymeric composition, and can be defined as:

$$\frac{1}{{R}_{hyd}} {=}^{def} \frac{1}{{N}^{2}} 〈 {\sum }_{i \neq j} \frac{1}{{r}_{ij}} 〉$$

wherein rᵢⱼ is the distance between two subparticles i and j, and the angular brackets represents an ensemble average over a collection of N subparticles. Hydrodynamic radius of a polymer can be mathematically calculated or measured using diffusion NMR, dynamic light scattering and others as will be understood by a person skilled in the art. The hydrodynamic radius of a polymer can be converted to radii of gyration using the Kirkwood-Riseman relationship (80-82) as will be understood by a person skilled in the art.

In some embodiments, the polymers composed in the polymeric composition have a “polydispersity index” or “dispersity” (PDI) of 1 to 20, determined by gel permeation chromatography (GPC), also referred to as size exclusion chromatography (SEC), as will be understood by a person skilled in the art. In this instance, PDI is defined as the weight average molecular weight (Mw) over the number average molecular weight (Mn), or PDI=Mw/Mn (99). In some embodiments, the PDI of the one or more polymers composed in the polymeric composition can also be determined by dynamic light scattering (DLS). The PDI will range from 0 to 0.7. If the PDI is greater than 0.7, the PDI of the polymers should be determined by an alternative method such as GPC, as will be understood by a person skilled in the art.

In the embodiments herein described, polymers herein used in suitable concentrations in polymeric compositions herein described are capable of controlling the structure of a hydrogel. The term “hydrogels” used herein refer to as crosslinked, three-dimensional networks of polymer chains that typically contain 80-99% water or aqueous solution filling the voids between polymer chains.

Biological hydrogels are hydrogels produced by a host individual or by bacteria in the host individual. The term “individual” as used herein includes a single biological organism wherein inflammation can occur including but not limited to animals and in particular higher animals and in particular vertebrates such as mammals and in particular human beings. Biological hydrogels typically surround biological functional entities such as cells, tissues, organs, or an entire organism and are in contact with or adhered to one or more cells possible organized in tissues and in particular an epithelial surface. The surface to which the biological hydrogels is attached can alter the physiochemical environment of the hydrogels by exchanging small molecules, solvent molecules, ions and additional compounds identifiable by a skilled person. In some embodiments, biological hydrogels establish and regulate the mechanical properties of cells and tissues and/or serve as lubricants in joints or on epithelial surfaces.

In biological hydrogel according to the present disclosure, the nature of the cross-linking connections among the polymer chains can be physical or chemical. The physical connections are weaker and more reversible compared to chemical connections. Polymer chains of hydrogels can be physically held together by electrostatic forces, hydrogen bonds, hydrophobic interactions or chain entanglements. The polymer chains of hydrogels can also be chemically connected by covalent bonds characterized by the sharing of pairs of electron between atoms, such as through disulfide bonds. There are also other types of non-specific chemical interactions among the polymer chains of the biological hydrogels, such as the hydrophobic-hydrophobic interaction. The combination of different types of crosslinking connections imparts complexity into the biological hydrogel structure.

In biological hydrogels according to the present disclosure, the crosslinked, three-dimensional networks of polymer chains comprise mostly protein-polysaccharide chains (i.e. glycoproteins). In particular, the polymer chains have protein backbones comprising amino acid sequences joined together. Thus, the polymer chains can adopt protein secondary structures such as alpha helices or beta sheets. The protein backbones can furthermore be attached with other molecules, such as polysaccharides. Examples of polymer strands composed in biological hydrogels include mucins, collagen, laminin, entactin and others as will be identified by a person skilled in the art upon reading of the present disclosure.

In biological hydrogels according to the present disclosure, the polymers can have linear, branched, hyperbranched, bottlebrush structures or combination thereof. For example, mucins comprise polymer strands having alternating hydrophobic, non-glycosylated region and hydrophilic, densely glycosylated bottlebrush region.

In some embodiments, biological hydrogels according to the present disclosure are associated with lipid bilayers. In particular, in some embodiments, the biological hydrogels are secreted by a mucous membrane, an epithelial tissue that lines body cavities and tubular organs of some individuals including the gut and respiratory passages of animals and in particular human beings.

In some embodiments, a biological hydrogel controlled by methods and systems herein described is a mucus or mucus layer lining and adhered to an epithelia.

The term “mucus” or “mucus layer” indicate a hydrogel layer rich in glycopeptide that coats wet epithelial surfaces in a body, including the oral cavity, airways, and gastrointestinal and urogenital tracts. Mucus is primarily composed of crosslinked, bundled, and entangled mucin fibers secreted by both goblet cells and the seromucinous glands of the lamina propria at the apical epithelium. Depending on the epithelia that mucus covers, the mucus thickness can vary between 10-700 μm, leading to different functions from a mechanical lubricant to a protective diffusion barrier. Mucin fibers, typically 10-40 MDa in size, are proteins glycosylated via proline, threonine, and/or serine residues by 0-linked N-acetyl galactosamine as well as N-linked sulfate-bearing glycans. Glycan coverage of mucins is dense, with 25-30 carbohydrate chains per 100 amino acid residues, and contributes up to 80% of the dry weight of mucus. Most mucin glycoproteins have a high sialic acid and sulfate content, which leads to a negative surface that increases the rigidity of the polymer via charge repulsion. The chemical content of the mucin fibers has been suggested to be correlated to mucus viscosity and elasticity. In some individuals, a mucus layer provides a lubricant between different tissues or organs of the individuals, protects body against pathogens as well as aids in the absorption of nutrients to facilitate their uptake by the epithelium. The term “epithelia” used herein refers to continuous sheets of cells, one or more layers thick, that cover the exterior surfaces of the body and line internal hollow organs that communicate with the outside environment such as the alimentary, respiratory and genitourinary tracts. Exemplary types of mucus comprise colonic mucus, cervicovaginal mucus, airway mucus, and other types of mucus as will be identified by a person skilled in the art. In particular, colonic mucus hydrogel refers to a mesh network of mucin proteins cross-linked either via reversible or irreversible chemical bonds or physical entanglements that is produced by the epithelial cells on the surface of a host colon.

In some embodiments, the biological hydrogels include extracellular matrix (“ECM”). The term “extracellular matrix” is a collection of extracellular molecules secreted by cells that provide structural and biochemical support to the surrounding cells. Cell adhesion, cell-to-cell communication and differentiation are common functions of the ECM. ECM can refer to a mesh network of collagen, laminin, entactin, elastin, fibronectin and other proteoglycans and glycoproteins that are crosslinked either via reversible or irreversible chemical bonds or physical entanglements that is found in connective tissue, tumors and basement membranes throughout mammalian host.

The extracellular matrix in animal includes the interstitial matrix and the basement membrane. Interstitial matrix is present between various animal cells, filled with gels of polysaccharides and fibrous proteins, which acts as a compression buffer against the stress placed on the ECM. Basement membranes are sheet-like depositions of ECM on which various epithelial cells rest. Each type of connective tissue in animals has a type of ECM: collagen fibers and bone mineral comprise the ECM of bone tissue; and reticular fibers and ground substance comprise the ECM of loose connective tissue.

In some embodiments, the biological hydrogels are biofilm extracellular polymeric substance. The term “biofilm” used herein indicates an aggregate of microorganisms in which cells adhere to each other on a surface. These adherent cells are frequently embedded within a self-produced matrix of extracellular polymeric substance (EPS). Biofilm extracellular polymeric substance is a polymeric conglomeration generally composed of extracellular DNA, proteins, and polysaccharides. Biofilms can form on living or non-living surfaces and can be found in natural, industrial and hospital settings.

In biological hydrogels according to the present disclosure the structure of the biological hydrogel and related modifications can be characterized by physiochemical parameters comprising molecular weight of the polymer chain forming the hydrogel, the mesh size (pore size), rheological measurements including viscosity (resistance to flow) and elasticity (stiffness), average number of Kuhn segments, the polymer volume fraction of the hydrogel in a swollen state and additional parameters identifiable by a skilled person.

In some embodiments, the structure of biological hydrogels according to the present disclosure, and related modifications can be characterized, for example, by viscoelasticity. In particular, the term “viscoelasticity” refers to the properties of the hydrogel that exhibits both viscous and elastic characteristics when undergoing deformation, such as compression or decompression, in responding to external stresses. The viscous characteristics refer to the hydrogel's resistance to flow and the elastic characteristics describe the ability of the hydrogel to return to the original shape upon removal of the stress. The viscoelastic properties of hydrogels can be measured by methods identifiable to a skilled person, such as rheological measurements (e.g. measurement of the storage and loss moduli of the hydrogel).

In some embodiments the structure of biological hydrogels according to the present disclosure, can be characterized by the related overall volume. The term “overall volume” referred to a biological hydrogel indicates the total volume occupied by the crosslinked polymer chains of the hydrogel and by a solvent filling the voids between the crosslinked polymer chains of the hydrogel. The overall volume of a hydrogel can be detected by various techniques identifiable by a skilled person such as microscopy and in particular by depositing microparticles of size larger than the hydrogel mesh size on the hydrogel surface, measuring the thickness between these and the epithelial surface on the other surface of the hydrogel and converting the thickness to the overall volume as well as additional techniques identifiable by a skilled person.

In some embodiments, the structure of biological hydrogels, according to the present disclosure, can be characterized by the related thickness. The term “thickness” referred to a biological hydrogel indicates the smallest of three dimensions of the biological hydrogel typically referred to the distance between opposing surfaces in a hydrogel layer. For example, in a mucous layer lining or adhered on an epithelium, the thickness of the mucous is the distance between the surface of the mucous adhered to the ephithelia and the surface of the mucous presented to the outside environment such as intestinal lumen and pathways of bronchi, lungs and female genital tract.

In some embodiments, the structure of biological hydrogels according to the present disclosure can be characterized by mesh size of the hydrogel. The term “mesh size” of biological hydrogels refers to the average distance between distinct polymer strands forming the biological hydrogels. The mesh size provides a measure of the space available between the chains of macromolecules comprised in the hydrogels. The mesh size can be determined theoretically or through the use of a variety of experimental techniques. In cases where the total polymer concentration and the length of individual polymer strands are known, this mesh size can be estimated mathematically. The mesh size can also be obtained from the analysis of electron micrographs, or using probe particles of difference size and monitoring with established techniques (e.g. fluorescence microscopy) whether the probes either penetrate or are excluded from the hydrogel, as well as other techniques as will be understood by a person skilled in the art.

In some embodiments, in biological hydrogels herein described, the average mesh size varies, ranging from 10 to 5000 nm. For example, the average mesh size of airway mucus ranges from 100 to 1440 nm. The average mesh size of murine colonic mucus ranges from 100 to 250 nm. The average mesh size of human cervicovaginal mucus ranges from 10 nm to greater than 500 nm. The variance in the mesh size of the biological hydrogels is related to the divergent permeability ability of different hydrogels.

In embodiments herein described, the mesh size of a biological hydrogel imparts to the hydrogel selective permeability properties which allow the hydrogel to form in the individual selective barriers that control the exchange of molecules between different compartments. For example, extracellular hydrogels externally coating plasma membrane of cells can prevent molecules or microscopic particles such as viruses or bacteria from reaching the plasma membrane. In those embodiments, the permeability properties of biological hydrogels allow the hydrogels to selectively filter molecules by size, also referred to as size filtering. In those embodiments, the mesh size of the hydrogel defines a molecular size cut-off. Molecules with a size smaller than the cut-off size are allowed to pass while molecules with a size larger than the cut-off size are rejected.

In some embodiments, selectively filtering of biological hydrogels herein described is affected by surface properties, through a process also referred to as interaction filtering. In those embodiments, molecules that engage in strong binding interactions with the hydrogel polymers become trapped in the hydrogel matrix independent of their size. The types of interactions between hydrogel polymers and the molecules can include electrostatic, hydrophobic interactions, hydrogen bonding, and other specific binding interactions.

In embodiments herein described, parameters that can be used to characterize the structure of a biological hydrogel according to methods and systems of the disclosure are interconnected one to the other as will be understood by a skilled person. For example, the polymer volume fraction indicates the volume of polymers/overall hydrogel volume fraction, the average number of Kuhn segments is indicative of the mesh size of a hydrogel in that the mesh size is proportional to (number of Kuhn segments) ̂(⅗), and the elasticity is related to volume fraction and number of Kuhn segments via Eq 7 herein described as will be understood by a skilled person. In another example, in a biological hydrogel layer such as mucus, the volume is equal to thickness multiplied cross sectional area as will be understood by a skilled person.

In some embodiments, the structure of a biological hydrogel can be controlled by contacting the hydrogel with one or more polymers herein described selected to have an averaged molecular weight and a concentration which are associated with a set structure of the biological hydrogel. In particular, the one or more polymers to be contacted with the biological hydrogel, the related averaged molecular weight and concentration, can be selected to provide the biological hydrogel with set structural features of the biological hydrogel, such as overall volume, mesh size and/or thickness, thus controlling the structure of the biological hydrogel.

“The term “contact” and “contacting” as used herein indicate an association between items resulting in a physical, chemical and/or biological interaction between the referenced items. In particular the term contact indicates the states or condition of physical touching as well as immediate proximity between referenced items including a distance allowing the referenced items to engage in one or more chemical and/or biological interactions.

In several embodiments, control of the structure of a biological hydrogel results in a compression or decompression of the biological hydrogel with respect to an original state before the contacting of the selected one or more polymers.

The term “compression” and “decompression” of the hydrogels respectively refers to a decrease or increase in the overall volume, the mesh size or the thickness of the biological hydrogels. In particular, a compression refers to a reduction in the overall volume of a hydrogel and decompression refers to an increase in the overall volume of the hydrogel.

In some embodiments, a reduction in the overall volume of the hydrogel results in the reduction in the thickness and the mesh size of the hydrogel, and therefore to a decreased thickness and a decreased mesh size. Consequently, the reduction in the mesh size of the hydrogel can further block the entrance of certain molecules and/or microscopic particles such as viruses or bacteria from passing through the hydrogels, thus preventing them from reaching the epithelial membrane.

In some embodiments, detection of a change in overall volume can be obtained by a direct measurement of change in volume, a measurement of change in the volume fraction taken up by the polymer chains composed in the hydrogel, a measurement of change in thickness and/or mesh size of the hydrogel. In embodiments where modification of biological hydrogel structure results in a compression of the biological hydrogel, the volume, thickness, and mesh size of the hydrogel decreases and the volume fraction taken up by the polymer chains composed in the hydrogel increases. In embodiments where modification of biological hydrogel structure results in a decompression of the biological hydrogel, the volume, thickness, and mesh size of the hydrogel increases and the volume fraction taken up by the polymer chains composed in the hydrogel decreases.

In some embodiments, detection of compression and/or decompression of a biological hydrogel can be performed by detecting structural features of the biological hydrogel before and after contracting the biological hydrogel with the one or more polymers. For example, the thickness of the biological hydrogel layer can be measured by microscopy such as confocal fluorescence microscopy, confocal reflectance microscopy or two-photon microscopy as will be recognized by a person skilled in the art (see Examples 2-5). The thickness of the biological hydrogel layer refers to the mean distance between the epithelial surface and the external surface of the hydrogel. In another example, detection of structural features of a biological hydrogel can be performed by taking an explant from a host, hydrating the explant with a saline solution, and adding probes to the hydrated explants, then measuring the separation between the probes excluded from the hydrogel and the host tissue as the thickness or volume of the hydrogel (see Examples 6-8).

In a further example, detection of structural features of biological hydrogel can be performed by taking explants from the host, covering them with a material that is immiscible with water and the hydrogel and capable of preserving the natural hydration of the hydrogel (e.g. fluorocarbon oil) and imaging the interface between the material and the surface of the tissue to measure the thickness or volume of the hydrogel (see Example 5).

In another example, detection of structural features of biological hydrogel can be performed by taking an explant from the host, fixing the explant in a preservative and imaging the hydrogel in the explant to measure the hydrogel volume, thickness, mesh size, or volume fraction (see Example 6)

In a further example, detection of structural features of biological hydrogel can be performed by taking a tissue from the host, fresh-freezing the tissue and imaging the hydrogel in the tissue to measure the hydrogel volume, thickness, mesh size, or volume fraction.

In some embodiments, control of an overall volume of a biological hydrogel provided by contacting the biological hydrogel with one or more polymers of a molecular weight from 100 Da to 5 MDa at a concentration from 0.05-80% w/v, the molecular weight and concentration selected to obtain a change in the overall volume of the biological hydrogel according to a Flory-Huggins model. The molecular weight and concentrations of the polymeric composition are referred to the molecular weight and concentration at the target site of the biological hydrogel.

In those embodiments, a Flory-Huggins model can be numerically solved for one or more set of overall volumes of the biological hydrogel and one or more polymers herein described. A percent of compression or decompression can be identified based on the numeric solution of the Flory-Huggins mode. A look-up table can then be provided connecting the one or more set of overall volumes of the biological hydrogel, the percent compression and/or the percent decompression with molecular weights and concentrations of the one or more polymers herein described. In particular, the look-up table can be provided based on parameters of the Flory-Huggins model related to the one or more polymers and associated with the one or more set overall volumes of the biological hydrogel in the numerically solved Flory-Huggins model. A specific combination of concentrations and molecular weights of the polymer corresponding to a specific set overall volume, percent compression and/or percent decompression can then be selected to provide a specific polymeric composition herein described. The polymeric composition can then be contacted to the biological hydrogel possibly replacing a preexisting composition in contact with the biological hydrogel to provide the biological hydrogel with the specific overall volume thus controlling the structure of the biological hydrogel.

A Flory-Huggins model used in embodiments herein described provides a mathematic model describing the free energy of mixing of polymers with solvent. The model considers the free energy of mixing (AG) in terms of two contributions, namely, the enthalpy of mixing (AH) and the entropy of mixing (AS). The entropy of mixing is determined by the volume fractions of solvent and polymer, whereas the enthalpy of mixing is determined by the Flory-Huggins interaction parameter x that gives a measure of the interaction of the polymer with the solvent molecules as well as the polymer-polymer interaction. In particular, the Flory-Huggins model of embodiments herein described can be implemented based on a mean-field theory, in which the interactions between molecules are assumed to be due to the interaction of a given molecule and an average field due to all the other molecules in the system. To aid in modeling, the solution is divided into a set of cells within which molecules or parts of molecules can be placed, also referred to as lattice model.

In Flory-Huggins models used in embodiments herein described, the polymer strands composed in the hydrogel network are treated as semi-flexible filaments. The supramolecular structuring such as stratified layers is not considered and it is also assumed that the hydrogel is isotropic. For simplicity, the electrostatic effects are also not considered in the Flory-Huggins model used herein given that in physiological solution, the Debye screening length is low (<1 nm).

In Flory-Huggins model used in embodiments herein described, the free energy cost of swelling a hydrogel network in a solution of free polymer is described using the elastic free energy, which accounts for deformations of individual network strands, and the free energy of mixing the polymer and solvent with the hydrogel. To describe the elastic free energy, a simple model based on the classical theory of rubber elasticity is used, which assumes affine deformations of the hydrogel network.

In particular, in the Flory-Huggins model used in embodiments herein described, the numerical calculations can be performed on the following four equations:

$$\begin{matrix}\frac{{μ}_{S}^{i n}}{RT} = \frac{1}{{N}_{M}} ( {v}_{M}^{1 / 3} {v}_{M}^{{0}^{2 / 3}} - \frac{{v}_{M}}{2} ) + ln {v}_{S}^{i n} + 1 - {v}_{S}^{i n} - \frac{{v}_{P}^{i n}}{y} + ( {χ}_{SM} {v}_{M} + {χ}_{SP} {v}_{P}^{i n} ) ( 1 - {v}_{S}^{i n} ) - {χ}_{MP} {v}_{M} {v}_{P}^{i n} & ( Eq . 1 ) \\ \frac{{μ}_{S}^{out}}{RT} = ln ( 1 - ϕ ) + ϕ ( 1 - \frac{1}{y} ) + {χ}_{SM} {ϕ}^{2} & ( Eq . 2 ) \\ \frac{{μ}_{P}^{i n}}{yRT} = \frac{1}{{N}_{M}} ( {v}_{M}^{1 / 3} {v}_{M}^{{0}^{2 / 3}} - \frac{{v}_{M}}{2} ) + \frac{1}{y} ln {v}_{P}^{i n} + \frac{1}{y} ( 1 - {v}_{P}^{i n} ) - {v}_{S}^{i n} + ( {χ}_{SP} {v}_{S}^{i n} + {χ}_{MP} {v}_{M} ) ( 1 - {v}_{P}^{i n} ) - {χ}_{SM} {v}_{S}^{i n} {v}_{M} & ( Eq . 3 ) \\ \frac{{μ}_{P}^{out}}{yRT} = \frac{1}{y} ln ϕ - 1 + ϕ + \frac{1}{y} ( 1 - ϕ ) + {{χ}_{SM} ( 1 - ϕ )}^{2} & ( Eq . 4 )\end{matrix}$$

wherein Vᵢ is the volume fraction of species i and xᵢⱼ is the Flory-Huggins interaction parameter between species i and j; here, solvent, mucus and free polymers are denoted as i=S, M, P, respectively. v_{M} is the hydrogel volume fraction, v_{M}⁰ is the hydrogel volume fraction in its initial preparation state, Vₛ is the molar volume of the solvent, N_{M} is the average number of Kuhn segments of the hydrogel network strands, which are the stiff segments making up each hydrogel network strand, y is the number of Kuhn segments of a polymer molecule composed in the polymeric composition, and φ is the volume fraction of the polymer in the polymeric composition, ranging from 0 to 1. It is also approximately the volume fraction of the polymer external to the hydrogel, assuming that the total volume of the hydrogel is much smaller than the volume of the polymer composition. The numerical calculations are performed on these equations at thermodynamic equilibrium, where μₛⁱⁿ=μₛ^{out} and μₚⁱⁿ=μₚ^{out}. Eq. 1-4 are also subject to the constraints v_{S}ⁱⁿ+v_{M}+v_{P}ⁱⁿ=1 and v_{S}^{out}+φ=1.

At polymer-free case (i.e. φ=0), which describes the initial swollen state of the mucus hydrogel, the system is described by Eq. 1 with μ_{S}ⁱⁿ=μₛ^{out}=0 and v_{P}ⁱⁿ=0, indicating there is no polymeric composition in the hydrogel and the chemical potentials of the solvent are equal inside and outside of the hydrogel network at equilibrium. Thus, a relationship between v_{M}⁰, χ_{SM}, N_{M}, and the mucus volume fraction in this initial swollen state, denoted as v_{M}^{s} can be defined as:

$$\frac{1}{{N}_{M}} ( {v}_{M}^{{S}^{1 / 3}} {v}_{M}^{{0}^{2 / 3}} - \frac{{v}_{M}^{S}}{2} ) + ln ( 1 - {v}_{M}^{S} ) + {v}_{M}^{S} + {χ}_{SM} {v}_{M}^{{S}^{2}} = 0 \qquad ( Eq . 5 )$$

The parameters N_{M}, χ_{SM}, v_{M}⁰, v_{M}^{s}, that characterize the swollen hydrogel (before exposure to polymer) can each be separately measured or calculated, while satisfying the constraint given by Eq. 5. A user can also measure or calculate three of them separately, and then calculate the fourth using Eq. 5.

Then, the case with added polymer (0<φ<1) is considered. Solving Equations 1-4 for v_{M} , with Eq 1=Eq 2 and Eq 3=Eq 4 at equilibrium, yields:

$$\begin{matrix}\frac{1}{{N}_{M}} ( {v}_{M}^{1 / 3} {v}_{M}^{{0}^{2 / 3}} - \frac{{v}_{M}}{2} ) + ln ( 1 - {v}_{M} - {v}_{P}^{i n} ) + {v}_{M} + {v}_{P}^{i n} - \frac{{v}_{P}^{i n}}{y} + ( {χ}_{SM} {v}_{M} + {χ}_{SP} {v}_{P}^{i n} ) ( {v}_{M} + {v}_{P}^{i n} ) - {χ}_{MP} {v}_{M} {v}_{P}^{i n} = ln ( 1 - ϕ ) + ϕ ( 1 - \frac{1}{y} ) + {χ}_{SM} {ϕ}^{2} & ( Eq . 6 ) \\ \frac{1}{{N}_{M}} ( {v}_{M}^{1 / 3} {v}_{M}^{{0}^{2 / 3}} - \frac{{v}_{M}}{2} ) + \frac{1}{y} ln {v}_{P}^{i n} + \frac{1}{y} ( 1 - {v}_{P}^{i n} ) - ( 1 - {v}_{M} - {v}_{P}^{i n} ) + ( {χ}_{SP} \cdot ( 1 - {v}_{M} - {v}_{P}^{i n} ) + {χ}_{MP} {v}_{M} ) ( 1 - {v}_{P}^{i n} ) - {χ}_{SM} ( 1 - {v}_{M} - {v}_{P}^{i n} ) {v}_{M} = \frac{1}{y} ln ϕ - 1 + ϕ + \frac{1}{y} ( 1 - ϕ ) + {{χ}_{SM} ( 1 - ϕ )}^{2} & ( Eq . 7 )\end{matrix}$$

In Eqs. 6-7, both the hydrogel volume fraction v_{M} and the concentration of polymer that goes inside the hydrogel v_{P}ⁱⁿ are unknown. To determine the level of compression for a given amount of added polymer φ, the user needs to numerically solve the combination of Eqs. 6-7 for these two unknowns v_{M} and v_{P}ⁱⁿ, inputting the values of φ and the other parameters that characterize the hydrogel in question—N_{M}, v_{M}⁰, y, χ_{SM}, χ_{SP}, χ_{MP}—into Eqs 6-7. The user can then calculate the percentage of compression as defined as:

Compressicn %=100%×(1−v_{M}^{s}/v_{M})

In some embodiments, the compression of a biological hydrogel upon in contact with a polymeric compositions both characterized by particular parameters can be calculated by numerically solving Eq. 6-7, wherein the compression is a reduction in the hydrogel volume less than or equal to 90% of its original volume.

In some embodiments, the polymers composed in a polymeric composition have a molecular weight in a range from 100 Da to 5 MDa with a number of Kuhn segments from 1 to 1000. The polymeric composition solution is provided at a concentration from 0.05-80% w/v, particularly from 0.05-20% w/v or 30-70% w/v or 65-70% w/v depending on the molecular weight of the polymers. In particular, for polymers having a number of Kuhn segment equal to 1, the polymers can have a molecular weight of 400 Da at a concentration from 65-70% w/v. For polymers having a number of Kuhn segment equal to 4, the polymers can have a molecular weight of about 6 kDa at a concentration from 30-70% w/v. For polymers having a number of Kuhn segment equal to 146, the polymers can have a molecular weight of about 200 kDa at a concentration from 0.05-20% w/v.

In some embodiments, the biological hydrogels have a number of Kuhn segments in a range from 20 to 10,000. The hydrogel volume fraction in its initial preparation state (v⁰ₘ) is in a range from 0.05-1. The Flory-Huggins interaction parameters between the hydrogel and the solvent (χₛₘ), between the hydrogel and the polymer of the polymeric compositions (χₘₚ) and between the solvent and the polymers of the polymeric compositions (χₛₚ) are in a range of −0.2-0.5, 0-0.5 and 0-0.5, respectively. Flory-Huggins interaction parameters can be experimentally measured by fitting scattering intensity profiles from small-angle neutron scattering, by fitting contact angle profiles of polymer blends, measuring interfacial width between homopolymer layers of different species using neutron reflectometry or inverse gas chromatography as will be understood by a person skilled in the art.

In some particular embodiments, polymers composed in a polymeric composition with a molecular weight of 400 Da, 6 kDa, and 200 kDa have a number of Kuhn segment of 1, 4, and 146 respectively. In some of these embodiments, a compression of a hydrogel can be obtained by providing the polymeric composition comprising such polymers at a concentration of 65-70% w/v for y=1, at a concentration of 10-70% w/v for y=4 and at a concentration of 0.05-20% w/v for y=146, when the other parameters in Eq. 6-7 are set as follows: Nₘ=999, Xₛₘ=0, v⁰ₘ=0.13, v^{s}ₘ=0.01, and Xₛₚ=0.45, Xₘₚ=0.3, or Nₘ=628, Xₛₘ=0, v₀ₘ=0.07, vₛₘ=0.01, χₛₚ=0.45, and Xₘₚ=0.3 or Nₘ=2026, Xₛₘ=0, v⁰ₘ=0.35, v^{s}ₘ=0.01, Xₛₚ=0.45, Xₘₚ=0.3.

In some of these embodiments, a compression of a hydrogel can be obtained by providing the polymeric composition comprising such polymers at a concentration of 55-70% w/v for y=1, at a concentration of 10-70% w/v for y=4, and at a concentration of 0.05-20% w/v for y=146 when the other parameters in Eq. 6-7 are set as follows: Nₘ=715, Xₛₘ=−0.2, v⁰ₘ=0.13, v^{s}ₘ=0.01, Xₛₚ=0.45, and Xₘₚ=0.3.

In some of these embodiments, a compression of a hydrogel can be obtained by providing the polymeric composition comprising such polymers at a concentration of 25-70% w/v for y=4 and at a concentration of 0.05-20% w/v for y=146 Nₘ=9425, Xₛₘ=0.45, ^{v0m}0.13, v^{s}ₘ=0.01, Xₛₚ=0.45, and Xₘₚ=0.3. In these embodiments, the compression is not obtained for polymeric composition comprising polymers having y=1.

In some of these embodiments, a compression of a hydrogel can be obtained by providing the polymeric composition comprising such polymers at a concentration of 10-70% w/v for y=4 and at a concentration of 0.05-20% w/v for y=146 when Nₘ=1247, Xₛₘ=0.1, vhu 0ₘ=0.13, v^{s}ₘ=0.01, Xₛₚ=0.45, and Xₘₚ=0.3. In these embodiments, the compression is not obtained for polymeric composition comprising polymers having y=1.

In some of these embodiments, a compression of a hydrogel can be obtained by providing the polymeric composition comprising such polymers at a concentration of 50-70% w/v for y=1, at a concentration of 10-70% w/v for y=4, and at a concentration of 0.05-20% w/v for y=146 when Nₘ=833, Xₛₘ=−0.1, vₘ⁰=0.13, v^{s}ₘ=0.01, Xₛₚ=0.45, Xₘₚ=0.3.

In some embodiments, polymers composed in a polymeric composition with a molecular weight of 400, 6 k, and 200 k have a number of Kuhn segment of 1, 2, and 76 respectively. In some of these embodiments, a compression of a hydrogel can be obtained by providing the polymeric composition comprising such polymers at a concentration of 65-70% w/v for y=1, at a concentration of 30-70% w/v for y=2 and at a concentration of 0.05-20% w/v for y=76, when Nₘ=1000, Xₛₘ=0, v⁰ₘ=0.13, v^{s}ₘ=0.01, Xₛₚ=0.45, and Xₘₚ=0.3.

In some embodiments, polymers composed in a polymeric composition with a molecular weight of 400, 6 k, and 200 k have a number of Kuhn segment of 1, 11, and 611 respectively. In some of these embodiments, a compression of a hydrogel can be obtained by providing the polymeric composition comprising such polymers at a concentration of 65-70% w/v for y=1, at a concentration of 2-70% w/v for y=11 and at a concentration of 0.05-20% w/v for y=611, when Nₘ=1000, Xₛₘ=0, vₘ⁰=0.13, v^{s}ₘ=0.01, Xₛₚ=0.45, and Xₘₚ=0.3.

In some embodiments, polymers composed in a polymeric composition with a molecular weight of 400, 6 k, and 200 k have a number of Kuhn segment of 1, 4, and 146 respectively. In some of these embodiments, a compression of a hydrogel can be obtained by providing the polymeric composition comprising such polymers at a concentration of 25-70% w/v for y=4 and at a concentration of 0.05-20% w/v for y=146, when Nₘ=1000, Xₛₘ=0, v⁰ₘ=0.13, v^{s}ₘ=0.01, Xₛₚ=0.45, Xₘₚ=0. In these embodiments, the compression is not obtained for polymeric composition comprising polymers having y=1.

In some of these embodiments, a compression of a hydrogel can be obtained by providing the polymeric composition comprising such polymers at a concentration of 40-70% w/v for y=1, at a concentration of 5-70% w/v for y=4, and at a concentration of 0.05-20% w/v for y=146, when Nₘ=1000, Xₛₘ=0, v⁰ₘ=0.13, v^{s}ₘ=0.01, Xₛₚ=0.45, Xₘₚ=0.5.

In some of these embodiments, a compression of a hydrogel can be obtained by providing the polymeric composition comprising such polymers at a concentration of 15-70% w/v for y=4, and at a concentration of 0.05-20% w/v for y=146, when Nₘ=1000, Xₛₘ=0, v⁰ₘ=0.13, v^{s}ₘ=0.01, Xₛₚ=0.45, Xₘₚ=0.2. In these embodiments, the compression is not obtained for polymeric composition comprising polymers having y=1.

In some of these embodiments, a compression of a hydrogel can be obtained by providing the polymeric composition comprising such polymers at a concentration of 50-70% w/v for y=1, at a concentration of 5-70% w/v for y=4, and at a concentration of 0.05-20% w/v for y=146, when Nₘ=1000, Xₛₘ=0, v⁰ₘ=0.13, v^{s}ₘ=0.01, Xₛₚ=0.45, Xₘₚ=0.4.

A user can use the Flory-Huggins model to derive concentrations at which a compression can occur for polymers and biological hydrogels characterized by different Flory-Huggins interaction parameters (Xₛₚ and Xₘₚ). Eq. 5-7 are numerically solved, wherein Nₘ=1000, Xₛₘ=0, vhu 0ₘ=0.13 v^{s}ₘ=0.01, Xₛₚ and Xₘₚ are varied.

For example, Table 1-3 show the polymer concentration ranges (% w/v) for a particular Xₛₚ and Xₘₚ combination when the polymer has a number of Kuhn segment equal to 1, 4 or 146, respectively.

TABLE 1
y = 1

| | X_{MP} = 0 | X_{MP} = 0.3 | X_{MP} = 0.5 |
|---|---|---|---|
| X_{SP} = 0 | n/a | 30-70 | 20-70 |
| X_{SP} = 0.2 | n/a | 50-70 | 25-70 |
| X_{SP} = 0.45 | n/a | 65-70 | 40-70 |

TABLE 2
y = 4

| | X_{MP} = 0 | X_{MP} = 0.3 | X_{MP} = 0.5 |
|---|---|---|---|
| X_{SP} = 0 | 10-70 | 5-70 | 15-70 |
| X_{SP} = 0.2 | 15-70 | 5-70 | 5-70 |
| X_{SP} = 0.45 | 25-70 | 10-78 | 5-70 |

TABLE 3
y = 146

| | X_{MP} = 0 | X_{MP} = 0.3 | X_{MP} = 0.5 |
|---|---|---|---|
| X_{SP} = 0 | 0.05-20 | 0.05-20 | 0.05-20 |
| X_{SP} = 0.2 | 0.05-20 | 0.05-20 | 0.05-20 |
| X_{SP} = 0.45 | 0.05-20 | 0.05-20 | 0.05-20 |

The above values for the molecular weight and concentrations of the polymers composed in the polymeric composition can be subject to further constraints when the polymeric composition is administered to a human body. For example, the polymeric composition solution, when in contact with the hydrogel at the target site or in transit through the host to the target site, is characterized by a total osmotic pressure either isotonic or hypertonic to the blood plasma, i.e. less than the physiological osmotic pressure (0.74 MPa), in order to prevent osmotic diarrhea and mitigate osmotic dehydration of tissues, or osmotic stress on tissues or commensal micro-organisms. Detailed description on how such constrains can affect the above calculated values can be found in Examples 15 and 16.

Under such physiological threshold (i.e. <0.74 MPa), the polymers composed in a polymeric composition have a molecular weight in a range from 100 Da to 5 MDa with a number of Kuhn segments from 1 to 1000. The polymeric composition solution is provided at a concentration from 0.05-30 w/v, particularly from 0.05-20%w/v or 5-20% w/v depending on the molecular weight of the polymers. For polymers having a number of Kuhn segment of approximately 4, the polymers can have a molecular weight in a range of 6 k Da at a concentration from 5 to 20% w/v. For polymers having a number of Kuhn segment of approximately 146, the polymers can have a molecular weight in a range of 200 kDa at a concentration from 0.5 to 20% w/v.

The network strands of the biological hydrogels have a number of Kuhn segments in a range from 20 to 10,000. The hydrogel volume fraction in its initial preparation state (V⁰ₘ) is in a range from 0.05-1. The Flory-Huggins interaction parameters between the hydrogel and the solvent (Xₛₘ), between the hydrogel and the polymer of the polymeric compositions (Xₘₚ) and between the solvent and the polymers of the polymeric compositions (Xₛₚ) are in a range of −0.2-05, 0-0.5 an 0-0.5, respectively.

A user can once again use the Flory-Huggins model to derive concentrations at which a compression can occur for polymers and biological hydrogels characterized by different Flory-Huggins interaction parameters (Xₛₚ and Xₘₚ), by further imposing the physiological threshold, i.e. the total polymer osmotic pressure less than 0.7 MPa.

For example, under the physiological osmotic pressure constraint, Table 4-6 show the polymer concentration ranges (% w/v) for a particular Xₛₚ and Xₘₚ combination when the polymer has a number of Kuhn segment equal to 1, 4 or 146, respectively. It is assumed that the polymers used herein will have an osmotic pressure similar to that of PEG.

TABLE 4
y = 1

| | X_{MP} = 0 | X_{MP} = 0.3 | X_{MP} = 0.5 |
|---|---|---|---|
| X_{SP} = 0 | n/a | n/a | n/a |
| X_{SP} = 0.2 | n/a | n/a | n/a |
| X_{SP} = 0.45 | n/a | n/a | n/a |

TABLE 5
y = 4

| | X_{MP} = 0 | X_{MP} = 0.3 | X_{MP} = 0.5 |
|---|---|---|---|
| X_{SP} = 0 | 10-20 | 5-20 | 15-20 |
| X_{SP} = 0.2 | 15-20 | 5-20 | 5-20 |
| X_{SP} = 0.45 | n/a | 10-20 | 5-20 |

TABLE 6
y = 146

| | X_{MP} = 0 | X_{MP} = 0.3 | X_{MP} = 0.5 |
|---|---|---|---|
| X_{SP} = 0 | 0.05-20 | 0.05-20 | 0.05-20 |
| X_{SP} = 0.2 | 0.05-20 | 0.05-20 | 0.05-20 |
| X_{SP} = 0.45 | 0.05-20 | 0.05-20 | 0.05-20 |

In some embodiments, control of an overall volume, mesh size and/or thickness of a biological hydrogel having an elastic modulus can be provided by a method wherein polymeric compositions herein described control an osmotic pressure on a surface of the hydrogel.

The terms “elastic modulus” or “modulus of elasticity”, indicates a parameter which measures a hydrogel's resistance to being deformed elastically when a force is applied to the hydrogel. The elastic modulus is defined as the ratio of the stress to the strain:

$$E = \frac{σ}{ɛ}$$

wherein the stress σ is defined as force per unit applied to the hydrogel (in MPa), the strain ε is the elongation or contraction per unit length (unitless or %) and the elastic modulus is in MPa. The elastic modulus of a particular biological hydrogel can be either numerically determined using a stress-strain diagram or experimentally measured using established techniques such as rheometry as will be understood by a person skilled in the art. For example, the elastic modulus of the colonic mucus hydrogel is about 10-100 Pa. The elastic modulus of basement membranes is about 450 Pa.

The term “osmotic pressure” Π used herein refers to a minimum pressure that needs to be applied to a solution to prevent the inward flow of water across a semipermeable membrane. In embodiments herein described, the osmotic pressure refers to a pressure applied to a surface of the biological hydrogel, typically an external surface which is a surface presented for contact with an externally provided composition. In some embodiments, where the biological hydrogel is adhered to an epithelium, the biological hydrogel has a basal surface through which the biological hydrogel is adhered to an epithelial surface and an external surface opposite to the basal surface through which the biological hydrogel is in contact with an external environment, such as the polymeric composition provided to the biological hydrogel for structural regulation.

In embodiments herein described wherein control of an overall volume, mesh size and/or thickness of a biological hydrogel is performed by controlling an osmotic pressure on a surface of the hydrogel, the method comprises contacting the biological hydrogel with selected one or more polymers having a molecular weight from 100 Da to 5 MDa at a concentration from 0.05%-80% w/v. In particular, the molecular weight and the concentration are selected to modify the difference between the external osmotic pressure externally applied to an external surface of the biological hydrogel and the internal osmotic pressure internally applied to the external surface of the biological hydrogel, also referred to as “an osmotic pressure difference” herein in the current disclosure. An increased osmotic pressure difference results in a more compressed state while a decreased osmotic pressure difference results in a less compressed, i.e. more decompressed state. The molecular weight and concentrations of polymeric composition are referred to the molecular weight and concentration at the target site of the hydrogel.

In embodiments of the methods herein described, the modification of the osmotic pressure can be either an increase or a decrease, which corresponds to a relative compression or decompression, respectively. In biological hydrogels herein described, when the biological hydrogels are not under compression by the one or more polymer of the disclosure, a base state, the osmotic pressure difference is less than 10% of the elastic modulus of the biological hydrogel. Accordingly, an osmotic pressure difference greater than or equal to 10% of the elastic modulus of the biological hydrogel causes a compression relative to the base state. For example, for hydrogels having an elastic modulus equal to 100 Pa, the polymers can be selected such that the osmotic pressure external to the hydrogel is 10 Pa large than the osmotic pressure internal to the hydrogel. In some embodiments, an increased osmotic pressure difference results in a more compressed hydrogel, while a decreased osmotic pressure difference results in a less compressed, i.e. more decompressed hydrogel.

In a biological hydrogel environment, the osmotic pressure can be affected by a variety of factors, including the molecular weight, the concentration of the solution and others factors such as the hydrodynamic radius (or radius of gyration) of the polymeric composition in the solution as will be identified by a person skilled in the art.

Increase of the concentration and/or molecular weight of the polymeric composition can alter the osmotic pressure of the polymeric composition solution. For example, in the dilute regime, the osmotic pressure of the polymeric composition is inversely proportional to the molecular weight of the polymers in the composition. In addition, the osmotic pressure of the solution can also be affected by hydrodynamic radius of the polymeric composition in the solution. For example, polymeric compositions having the same molecular weight and concentration can have different osmotic pressure, in particular, when one is linear and the other is branched, indicating that the hydrodynamic radius also plays a role in determining the osmotic pressure. In some cases, polymers having the same hydrodynamic radius have approximately the same osmotic pressure at a given concentration.

In some embodiments, the one or more polymers composed in the polymeric composition are selected such that the polymers have a size larger than the hydrogel mesh size and therefore do not penetrate into the hydrogel. The size of the polymers is defined as 2× radius of gyration. In such cases, the polymer composition can induce compression by elevating the external osmotic pressure outside the hydrogel without affecting the internal osmotic pressure. It is assumed that the internal osmotic pressure is zero when the polymers of the polymeric composition do not penetrate into the hydrogel. Thus, the osmotic pressure difference is equal to the external osmotic pressure outside the hydrogel.

In some embodiments, to select the polymers capable of controlling the overall volume, mesh size and/or thickness of a hydrogel by controlling an osmotic pressure of a hydrogel, a user can determine the elastic modulus of the hydrogel (G) using established techniques such as rheometry as will be understood by a person skilled in the art. The user can also determine the average mesh size of the biological hydrogel using various approaches. For example, the user can take an explant from the host, hydrate it with a saline solution, add probes of different sizes and monitor with established techniques (e.g. fluorescence microscopy) whether the probes either penetrate or are excluded from the hydrogel, by fixing the explant in a preservative or fresh-freezing the hydrogel and then imaging it. The user can further select a polymer having a size larger than the average mesh size of the hydrogel determined in the previous step.

The user can further select the polymers providing a desired osmotic pressure Π, by providing a look-up table connecting one or more concentrations of one or more polymers herein described with one or more corresponding external osmotic pressures for the biological hydrogel and selecting from the look-up table the one or more concentrations of the one or more polymers associated with a desired external osmotic pressure.

In some embodiments, the external osmotic pressure, which is applied externally by a polymeric composition solution to the hydrogel (Π), can be determined given its concentration c. For example, the osmotic pressure can be experimentally measured by a user skilled in the art using techniques such as vapor pressure depression, freezing point depression, or membrane osmometry as will be understood by a person skilled in the art.

The osmotic pressures can also be mathematically calculated. For a given concentration c, the user firstly checks whether the concentration is above or below the polymer overlap concentration c*. The polymer overlap concentration can be experimentally determined using viscometry or light scattering. It can also be calculated using the following equation:

c*=M_{w}/(4πN_{A}R_{g}³/3) (Eq. 8)

where Mw is the molecular weight of the polymer, N_{A} is Avogadro's number, and R_{g} is the radius of gyration of the polymer.

If the polymeric composition has concentration c below c*, the user can calculate the external osmotic pressure using van't Hoff s law:

Π=cRT (Eq. 9)

where R is the gas constant and T is temperature.

If the polymeric composition has a concentration c above c*, the user can calculate the osmotic pressure using des Cloizeaux's law:

Π=αc^{9/4}RT (Eq. 10)

where a is an experimentally-determined prefactor.

In some embodiments, the user can then determine the extent of compression by calculating the percentage of compression defined as % compression=100×Π/G, wherein Π is the external osmotic pressure of the polymeric composition and G is the elastic modulus of the biological hydrogel.

In some embodiments, the polymers composed in the polymeric composition can have a size smaller than the hydrogel mesh size and therefore can diffuse into the hydrogel, thus lowering the osmotic pressure difference. In such cases, the osmotic pressure difference results from non-uniform portioning of the polymers between the hydrogel exterior and interior, for example, due to entropic effects of polymer confinement inside the hydrogel or enthalpic interactions with the components of the hydrogel.

To determine the osmotic pressure difference in embodiments where one or more polymers have a size smaller than the mesh size, the external osmotic pressure, internal osmotic pressure and/or the osmotic pressure difference can be determined by experimentally detecting, for a given amount of added polymer, the ratio between the concentration of the polymers inside the hydrogel and the concentration of the polymers outside the hydrogel, and then calculating or detecting the corresponding internal osmotic pressure, external osmotic pressure and/or the related osmotic pressure difference based on the detected concentration of the polymers inside the hydrogel and the concentration of the polymers outside the hydrogel.

In some of those embodiments, firstly the user experimentally determines a “calibration curve”, which shows, for a given amount of added polymer (φ) the ratio between the concentration of the polymers inside the hydrogel and the concentration of the polymers outside the hydrogel (v_{P}ⁱⁿ/φ). The volume of polymeric composition used must be larger than the volume of the hydrogel such that the concentration of the polymers outside is approximately equal to φ.

In some embodiments herein described, the calibration curve can be construed by experimentally depositing different formulations of the polymers of a set concentration (characterized by polymer volume fraction co) onto the hydrogel. This will be seeded with a dilute amount of the same polymer, but fluorescently-labeled. For example, the polymers can be labeled with FITC dye, and the fluorescently-labeled fraction “spiked” into the polymer formulation will be at a concentration of 0.05% w/v. The user can then use fluorescence microscopy to directly image the hydrogel. Alternatively, the user can sample the polymer solution from inside and outside the hydrogel and analyze their fluorescence separately. The user can then determine the fluorescence levels of the polymer solution inside (fᵢₙ) and outside (fₒᵤₜ) the hydrogel. Separately, the user can determine the relationship between the fluorescence level of the labeled polymer solution and its concentration, by preparing different formulations of the labeled polymer at different concentrations and measuring their fluorescence levels. The user can then use this relationship to calculate the concentrations of the dyed polymer solution inside (c_{P}ⁱⁿ) and outside (c_{P}^{out}) the hydrogel, and use these values to obtain v_{P}ⁱⁿ/φ=c_{P}ⁱⁿ/c_{P}^{out}. The user can repeat the above procedure for different values of φ, thereby constructing the calibration curve described in the previous paragraph quantifying the relationship between v_{P}ⁱⁿ/φ and φ. The user can then use this calibration curve to determine, for a given φ, the corresponding value of v_{P}ⁱⁿ.

In some embodiments herein described, the osmotic pressures inside and outside the hydrogel, Πⁱⁿ and Π^{out}, can then be determined using the values of v_{P}ⁱⁿ and φ, respectively. The osmotic pressures corresponding to each of these concentrations can be experimentally determined by a user skilled in the art using vapor pressure depression, freezing point depression, membrane osmometry or other established techniques. The osmotic pressures can also be mathematically calculated according to Eq. 8-9 as previously described.

In some embodiments of the methods and systems herein described, related molecular weight and concentrations of the one or more polymers are selected to provide an external osmotic pressure and/or an osmotic pressure difference resulting in a compression of the biological hydrogel. In those embodiments, the osmotic pressure Π or the osmotic pressure difference is greater than or equal to 10% of the hydrogel elastic modulus, resulting in compression of the hydrogel to a volume less than or equal to 90% of its original volume. In some of those embodiments, the molecular weight of the polymer can also be selected so that polymer size=2× radius of gyration>hydrogel mesh size; where the radius of gyration can be measured and is also proportional to (molecular weight)̂(⅗).

In some of those embodiments, the user can further determine the elastic modulus of the hydrogel (G) using techniques such as rheometry as will be understood by a person skilled in the art, and then calculate the percentage of compression defined as % compression=100×(Π^{out}−Πⁱⁿ)/G

In embodiments of the methods herein described, contacting the one or more polymers herein described with a biological hydrogel can be performed by depositing a solution containing polymers herein described onto an external surface of a biological hydrogel ex vivo such as a luminal surface of the a colonic mucouse (see e.g. Example 12). The term “ex vivo” refers to experiments or measurements carried out in or on tissue from an organism in an external environment with the minimum alteration of natural condition. Alternatively, the combining can be performed by administering the polymeric composition to a subject in vivo through various administration routes including oral ingestion, inhalation, intranasal, rectal or vaginal administration, topical application, intravenous or subcutaneous injections and others as will be recognized by a person skilled in the art. The polymeric composition to be administrated can be in a form of an aqueous solution, cream, solid powder, tablets, aerosols or other forms as will be understood by a person skilled in the art.

In the embodiments herein described, the one or more polymers composed in the polymeric composition solution can be further subjected to physiological constraints in an individual. When the polymeric composition is administered to an individual, the polymeric composition used herein can be ingested by the individual without inducing toxicity or adverse physiological effects. In some embodiments, the polymeric composition cannot be degraded by or within the individual. In some other embodiments, the polymeric composition can be degraded by or within the individual to achieve a desired concentration or length or molecular weight or hydrodynamic radius at a target location including the colon, intranasal, rectal, vaginal area or other alimentary, respiratory and genitourinary tracts.

In some embodiments, the one or more polymers composed in the polymeric composition solution are selected to create an osmotic pressure less than 100 MPa such that it does not induce osmotic diarrhea when it reaches the colon of the individual.

In some embodiments, the one or more polymers composed in the polymeric composition solution herein described are selected to have a total osmotic pressure less than 0.74 MPa at the target site of the biological hydrogel or in transit through a host until it reaches the target site of the biological hydrogel. Such target osmotic pressure threshold is chosen such that the polymeric composition, when contacting the hydrogel at the target sit or in transit through the host to the target site, is isotonic or hypertonic to blood plasma that has a physiological osmotic pressure of 0.74 MPa. Therefore, osmotic diarrhea in the intestinal diarrhea can be prevented and osmotic dehydration of tissues, or osmotic stress on tissues or commensal micro-organisms can be mitigated.

In some embodiments, a method to compress colonic mucus hydrogel under a physiological osmotic pressure is described. The method comprises contacting the colonic mucus hydrogel with one or more polymers selected to provide an osmotic pressure difference equal to or greater than 10% of the elastic modulus of the colonic mucus and a total osmotic pressure lower than the physiological osmotic pressure (0.74 MPa). The colonic mucus has an elastic modulus, a basal surface in contact with an epithelial cell and an external surface opposite to the basal surface in contact with an external environment. In the colonic mucus at a base state, an osmotic pressure difference between an external osmotic pressure externally applied to the external surface of the colonic mucus and an internal osmotic pressure internally applied to the external surface of the colonic mucus is less than 10% of the elastic modulus of the colonic mucus.

In such embodiments, the one or more polymers composed in the polymeric composition are selected to have a molecular weight greater than 200 kDa and at a concentration from 0.05 to 20% w/v, thus resulting in an osmotic pressure difference at the target site greater than or equal to 10% of the colonic mucus elastic modulus and a total osmotic pressure less than the physiological osmotic pressure (0.74MPa). The total osmotic pressure in these embodiments can be calculated according to the following equation:

Π^{total}=(1/Vₜₒₜ)*(Π^{out}*Vₑₓₜ+Πⁱⁿ*Vᵢₙₜ)

wherein Π^{total} is the total osmotic pressure; Π^{out} is the external osmotic pressure; Πⁱⁿ is the internal osmotic pressure; Vₑₓₜ is the volume of the polymeric composition outside the hydrogel; Vᵢₙₜ is the volume of the polymeric composition inside the hydrogel; and Vᵢₙₜ is the sum of Vₑₓₜ and Vᵢₙₜ .

Similar to other aspects previously described, a user can determine the elastic modulus of the colonic mucus (G) using techniques such as rheometry and the osmotic pressure (Π) either experimentally or theoretically as will be understood by a person skilled in the art.

In some embodiments, a method to decompress a compressed biological hydrogel is described. That is, the compressed biological hydrogel can be reversed to a less compressed, i.e. more decompressed state of the hydrogel having an increased overall volume, thickness and/or mesh size relative to the initial, compressed state. In some embodiments, the decompression can be performed by decreasing the osmotic pressure difference.

In some embodiments, decompressing a compressed hydrogel can be performed by decreasing the concentration of the polymeric composition or by removing the polymeric composition from the compressed biological hydrogel, such as by selectively rinsing the biological hydrogel with water. Alternatively, such reverse can be performed by degrading the polymeric composition to a different polymeric composition having a decreased molecular weight, chain length or hydrodynamic radius.

Accordingly, in some embodiments, compositions can be designed to comprise microbes, enzymes and/or other molecules capable of degrading the polymers together with an acceptable vehicle and in various formulations suitable to deliver the microbes, enzymes and/or other molecules to the hydrogel of interest. In those embodiments, one or more active agents can be comprised in amounts effective to degrade polymers when added to the polymeric composition and/or a biological hydrogel herein described. In those embodiments compositions herein described can be contacted with the polymeric composition applied to the hydrogel and/or to the hydrogel to decompress the hydrogel.

In some embodiments, the decompressing can be performed by adding microbes to the mixture of the biological hydrogel and the polymeric composition solution, wherein the microbes are capable of degrading the polymeric composition. In particular, the microbes degrade the polymeric composition into polymer fragments having a smaller molecular weight and hydrodynamic radius. Such composition change induced by microbes can cause a decreased osmotic pressure difference between the hydrogel and the polymeric composition solution.

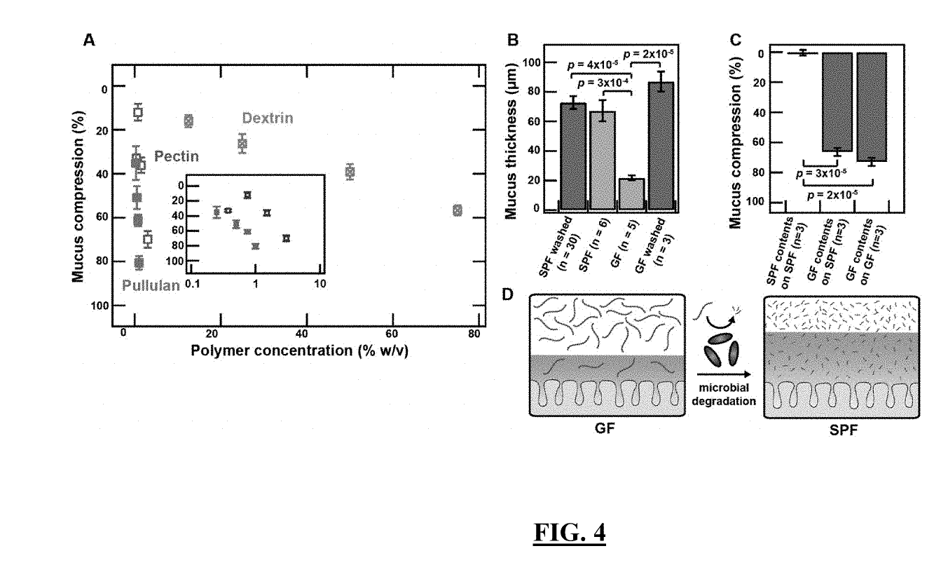

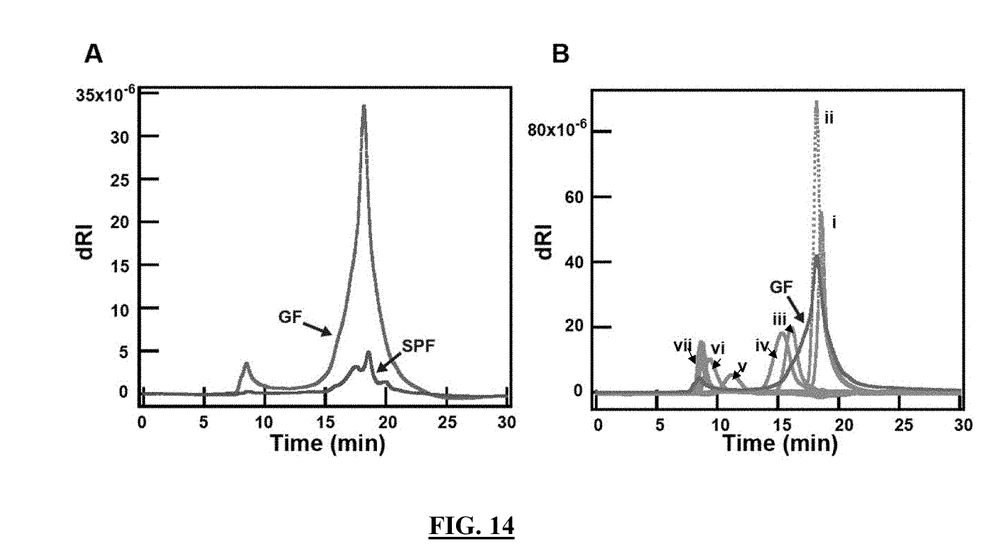

In some embodiments, the microbes, by modifying the polymeric composition of intestinal contents, can actively modulate the compression state of the colonic mucus hydrogel (see Example 14, FIGS. 4 and 14).

In some embodiments, the methods and compositions herein described can be used to decompress mucus hydrogels in the gastrointestinal system including the stomach, colon and small intestinal to promote colonization or to eliminate or disperse mucus-embedded pathogens such as Helicobacter pylori infection in the stomach, mucosal biofilms in small-intestinal bacterial overgrowth (SIBO) and cystic fibrosis (CF) or Clostridium difficile infection in the colon. In some of these embodiments, the decompression can be performed by adding polymer-degrading microorganisms or probiotics and/or polymer-degrading enzymes (including mucinases) to the mucus.

In some embodiments, the methods and compositions herein described can be used to decompress the cervicovaginal and vaginal mucus hydrogel to increase fertility and to aid sperm passage. In some of these embodiments, the decompression can be performed by adding polymer-degrading enzymes (including mucinases) or polymer-degrading microorganisms or probiotics to the cervicovaginal and vaginal mucus.

In some embodiments, the methods herein described can be used to decompress the mucus in the lower respiratory tract to promote mucociliary clearance in patients having CF, chronic obstructive pulmonary disease (COPD) or bronchial asthma. In some of these embodiments, the decompression can be preformed by administrating to the patient enzymes (including mucinases or DNases) in the form of solution aerosols or powder aerosols.

In some embodiments, the methods to control the structure of other types of biological mucus hydrogel, such as cervico-vaginal mucus, stomach mucus and tracheobronchial mucus, are described. The method comprises contacting the mucus hydrogel with one or more polymers of a molecular weight from 100 Da to 5 MDa at a concentration from 0.05-80% w/v, the molecular weight and the concentration of the one or more polymers selected to provide an osmotic pressure difference equal to or greater than 10% of the elastic modulus of the mucus hydrogel. In some embodiments, the polymeric composition solution containing the one or more polymers can be further subjected to physiological constraints in an individual, i.e. lower than the physiological osmotic pressure of 0.74 MPa. The mucus hydrogel has an elastic modulus, a basal surface in contact with an epithelial cell and an external surface opposite to the basal surface in contact with an external environment. In the mucus hydrogel at a base state, an osmotic pressure difference between an external osmotic pressure externally applied to the external surface of the mucus and an internal osmotic pressure internally applied to the external surface of the mucus is less than 10% of the elastic modulus of the mucus. In some particular embodiments, the one or more polymers in the polymeric composition comprise naturally derived polymers and/or synthetic polymers such as PEG.

In some embodiments, the biological mucus hydrogel is a cervico-vaginal mucus hydrogel having a mesh size of 340 ±70nm (100), an elastic modulus of about 100-300 Pa (101) and a chemical composition comprising MUC5AC, MUC5B, and MUC6 (102).

The mesh size of the cervico-vaginal mucus hydrogel can be controlled by the administration of chemical compositions. For example, it has been shown that before the administration of a detergent, the cervico-vaginal mucus hydrogel is permeable to objects smaller than 500 nm in diameter. After the administration of the detergent, the cervico-vaginal mucus hydrogel is impermeable to objects of 200-500 nm in diameter (101). It has also been shown that the cervico-vaginal mucus mesh can slow the diffusion of Herpes Simplex Virus as well as HIV virus-like particles by 10,000 fold compared to water.

In some of the embodiments herein described, a polymeric composition comprising one or more polymers of a molecular weight from 100 Da to 5 MDa at a concentration from 0.05-80%w/v can be used to control the mesh size of a biological mucus hydrogel such as the cervico-vaginal mucus hydrogel to selectively filter virus molecules by size. In particular, the polymeric composition can be administrated to an individual to obtain a desired molecular size cut-off. Molecules with a size smaller than the cut-off size can pass while molecules with a size larger than the cut-off size, such as Herpes Simplex Virus, HIV virus-like particles and other, are rejected.

In some embodiments, the biological mucus hydrogel is a tracheobronchial mucus hydrogel on top of the periciliary layer (“PCL”) having an elastic modulus of 2-80 Pa in dogs, rats and horses (106) with a chemical composition comprising primarily MUC5AC and MUC5B (103).

In some embodiments, the biological mucus hydrogel is a stomach mucus hydrogel having a mesh size larger than 1 micron (104), a thickness of about 80-150 microns in rats (105), an elastic modulus of about 3-30 Pa (106), and a chemical composition primarily comprising of MUCSAC, MUCSB, MUC6 and MUC2 (105).

In some embodiments, the one or more polymers herein described are comprised in a composition together with a suitable vehicle. The term “vehicle” as used herein indicates any of various media acting usually as solvents, carriers, binders or diluents for the one or more polymers that are comprised in the composition as an active ingredient. In some embodiments, vehicles herein described comprise diluents and excipients.

The term “excipient” as used herein indicates an inactive substance used as a carrier for the active ingredients of a medication. Suitable excipients for the pharmaceutical compositions herein described include any substance that enhances the ability of the body of an individual to absorb the one or more polymers or combinations thereof. Suitable excipients also include any substance that can be used to bulk up formulations with the peptides or combinations thereof, to allow for convenient and accurate dosage. In addition to their use in the single-dosage quantity, excipients can be used in the manufacturing process to aid in the handling of the peptides or combinations thereof concerned. Depending on the route of administration, and form of medication, different excipients can be used. Exemplary excipients include, but are not limited to, antiadherents, binders, coatings, disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluents include any substance that can decrease the viscosity of a medicinal preparation

The polymeric composition comprises in a suitable vehicle, one or more polymers of a molecular weight from 100 Da to 5 MDa at a concentration from 0.05-80% w/v, the one or more polymer the molecular weight and concentration selected to obtain a change in the overall volume, mesh size and/or thickness of the biological hydrogel according to methods herein described.

According to another aspect, a system to control a structure of a biological hydrogel is described. The system comprises one or more polymeric compositions herein described and a look-up table. In some embodiments, the look-up table connects one or more molecular weight and/or one or more concentrations of one or more polymers in the one or more polymeric compositions with at least one of the one or more overall volumes, one or more mesh size and one or more thicknesses of the biological hydrogel, the connection being established according to methods herein described. In other embodiments, the look-up table connects one or more molecular weight and/or one or more concentrations of one or more polymers in the one or more polymeric compositions with a percentage of compression and/or a percentage of decompression of the biological hydrogel according to methods herein described.

In embodiments described herein, the one or more polymeric compositions can be provided in various formulations alone or in combination with other components to compress a structure of a biological hydrogel.

In some embodiments, the biological hydrogel is a mucus in the gastrointestinal system, including stomach, colon, and small intestine. The polymeric composition can be formulated with dry/semi-dry/concentrated polymers (e.g., capsule with powder, chewable tablets, capsule with a liquid concentrate) that become rehydrated/dissolved in situ to a desired concentration with additional water intake or normal secretions. The polymers can also be provided as processed/solid food additives in quantities that will give a desired concentration assuming standard hydration of the food upon ingestion. The polymers can also be provided as non-alcoholic beverage additives at a desired concentration assuming standard water absorption/concentrating effect of the gastrointestinal system. The polymers can also be provided as additives to medical nutrition mixes (e.g., for administration through gastric tube), liquid diets and oral rehydration solutions in the absence of solid food intake for extended periods of time at a desired concentration to aid the mucosal barrier function. The polymers can also be provided as additives to infant formula and infant medical nutrition to aid the mucosal barrier properties (e.g., in premature babies, necrotizing enterocolitis (“NEC”)). The polymers can also be provided as additives to sports beverages and nutrition (e.g., gels, energy bars) to mitigate the effects of extensive physical loads/exercises and the overheating on the gastrointestinal barrier properties (107, 108, 109). In some embodiments, the polymers can also be provided by polymer (e.g. kefiran)-producing probiotics or in combination therewith, such as Lactobacillus bulgaricus and Lactobacillus kefiranofaciens to compress mucus (110, 111).

In embodiments wherein the biological hydrogel is a stomach mucus hydrogel, the polymers in the polymeric composition can be provided in combination with Helicobacter pylori eradication therapy such as antacid/antibiotics (for example, clarithromycin or metronidazole) and gastric protective agents (112) to inhibit the invasion of the pathogen through the mucus layer. The polymers can also be provided in combination with proton-pump inhibitors (PPIs) (for example, omeprazole) and other antisecretory drugs (for example, H2 receptor antagonists such as cimetidine and ranitidine) to aid the mucosal barrier function and to protect gastric mucosa, for example, from overgrowing microbes. The polymer can also be provided in combination with gastric protective agents (for example, sucralfate, bismuth compounds such as bismuth subsalicylate and bismuth subcitrate) to aid the treatment of ulcers in the upper gastrointestinal system. The polymers can also be provided in combination with antacids (for example, Na/Ca/Mg/Al-(bi)carbonate and Al/Mg-hydroxide) to aid the mucosal barrier function/to protect gastric mucosa. The polymers can also be provided in combination with mucolytic agents (for example, acetylcysteine or carbocysteine) given orally for respiratory therapy to aid the mucosal barrier function and to protect gastric mucosa. The polymers can also be provided in combination with nonsteroidal anti-inflammatory drug (NSAIDs) therapy, either enteral or parenteral, to protect gastric mucosa. The polymers can also be provided in combination with anti-GERD (gastroesophageal reflux disease) therapy to protect the tissue of Barrett's esophagus when mucus layer is present (113).

In embodiments wherein the biological hydrogel is a colonic mucus hydrogel, the polymeric composition can be formulated for oral administration or intrarectal administration. In cases of oral administration, the polymers can be formulated in tablets and capsules, in powder or concentrate, with an enteric coating for release in the lower gastrointestinal tract. The polymers can also be provided in combination with osmotic laxatives (for example, polymer-based osmotic laxatives such as PEG 3350 or salt-based osmotic laxatives such as Na-phosphate and Mg-hydroxide/sulfate/citrate or sugar-based osmotic laxatives such as lactulose) to aid the mucosal barrier function, to protect colonic mucus from swelling and to prevent microbial invasion. The polymers can also be provided in combination with antidiarrheal agents (for example, hygroscopic compounds such as carboxymethylcellulose, Ca-polycarbophil and Al/Mg-silicate, or bile acid sequestrants such as cholestyramine, colestipol and colesevelam) to aid the mucosal barrier function. The polymers can also be provided in combination with prokinetic agents (for example, metoclopramide or domperidone) to aid the mucosal barrier function. The polymers can also be provided in combination with antihelminthic drugs to inhibit the invasion of the parasites through the mucus layer. The polymers can also be provided in combination with antimicrobial drugs in the management of infectious colitis to inhibit the invasion of the pathogen through the mucus layer. The polymers can also be provided in combination with anti-IBD (inflammatory bowel disease) drugs (for example, sulfasalazine) to aid the mucosal barrier function. The polymers can also be provided in combination with anti-IBS (irritable bowel syndrome) drugs (for example, lubiprostone) to aid the mucosal barrier function. The polymers can also be provided in combination with gastrointestinal replacement therapies (for example, pancreatic enzymes or bile acids) to aid the mucosal barrier properties.

In cases of intrarectal administration, the polymers can be provided as additives to enema solutions to aid the mucosal barrier function, to protect colonic mucus from swelling and to prevent microbial invasion. The polymers can also be provided in combination with anti-IBD drugs with intrarectal route of administration such as Cortifoam® enema or foam. The polymers can also be provided in combination with fecal microbiota transplantation (FMT) to aid the mucosal barrier function, to protect colonic mucus from swelling and to prevent microbial invasion.

In some embodiments, the biological hydrogel is a cervicovaginal or vaginal mucus hydrogel. In such cases, the polymeric composition comprising the polymers herein described can be used to prevent or decrease the transmission of STDs of viral and microbial origin or as a birth control. The polymers can be formulated in various forms of local delivery such as gels, foams, creams, suppositories or film, prepared with a desired concentration or very little rehydration by natural secretions including semen. The desired concentration is selected according to the look-up table. The polymers can also be provided in combination with personal lubricants, with barrier contraceptives (such as condoms, diaphragms, sponges, etc.) or with spermicide compounds. In some embodiments, the polymers can also be provided by polymer-producing probiotics or in combination therewith to compress the cervicovaginal or vaginal mucus. Examples of polymer-producing probiotics include Lactobacillus crispatus that has shown to reduce HIV transport through CVM and can produce exopolysaccharide against Candida albicans (114).

In some embodiments, the polymeric composition herein described can be administrated to an individual to prevent or to decrease the transmission of airborne infections or the exposure to air pollution and air-borne allergens. In particular, the polymeric composition can be provided in solution aerosols formulated at a desired concentration, or in aerosols of concentrated polymer solution or powder, or further in combination with anti-asthma compounds such as bronchodilators or anti-inflammatory compounds.

Further effects and characteristics of the present disclosure will become more apparent hereinafter from the following detailed disclosure of by way of illustration only with reference to an experimental section.

### EXAMPLES

The methods and system herein disclosed are further illustrated in the following examples, which are provided by way of illustration and are not intended to be limiting.

In particular, the following examples illustrate exemplary polymeric compositions with a polyether (PEG) and polysaccharides (dextrin, pectin and pullulan) and related exemplary methods and systems to regulate volumes of exemplary hydrogels such as colonic mucus hydrogel.

A person skilled in the art will appreciate the applicability and the necessary modifications to adapt the features described in detail in the present section, to additional polymeric composition, hydrogels and to other compositions, methods and systems according to embodiments of the present disclosure.

### Example 1

### Polymer Probes for Hydrogel Thickness Detection

The following polymers were used as probes (all in 1× PBS) to detect the adherent mucus hydrogel, to measure the mucus thickness, or to help quantify the mucus mesh size in the initial swollen state (see Examples 6 and 10 below): Methoxyl polyethylene glycol-FITC (mPEG-FITC, Nanocs, Boston, Mass., USA), weight averaged molecular weight 350, 1.2×10⁻² mg/mL; mPEG-FITC (Nanocs), weight averaged molecular weight 5 kDa, 3.3×10⁻² mg/mL; mPEG-FITC (Nanocs), weight averaged molecular weight 200 kDa, 0.6 mg/mL; FITC-dextran (Sigma-Aldrich, St. Louis, Mo., USA), average molecular weight 2 MDa, 0.1 mg/mL; Fluorescent polystyrene microparticles (micromer, from micromod GmbH, Rostock, Germany), coated with PEG 300 to render them chemically inert (39), 0.02-0.2% volume fraction of manufacturer-reported average diameters 100 nm, 250 nm, 500 nm, 1 μm, or 5 μm. Penetration measurements used fluorescently labeled polymers at concentrations below those that cause mucus compression.

Probes or polymers 500 nm or smaller were characterized using dynamic light scattering performed on 200-500 μL of each sample with a Wyatt Dynapro NanoStar instrument. The data were collected and analyzed using Wyatt DYNAMICS software 7.1. Hydrodynamic radii were determined by fitting the data using a regularization analysis. The wavelength of the laser was 658 nm and the scattering angle was 90°. The microparticle solutions were unfiltered, while the polymer solutions were filtered using either a 0.2 μm Fisherbrand (PEG 400, PEG 6 kDa, PEG 200 kDa, fluorescent PEG 200 kDa, fluorescent dextran 2 MDa, fluorescent PEG 5 kDa) or a 0.45 μm Puradisc (pullulan, dextrin) syringe filter. All samples were dispersed in 1× PBS, and the following concentrations or volume fractions were used: 3 mg/mL (fluorescent PEG 200 kDa), 1 mg/mL (fluorescent dextran 2 MDa), 0.1% v/v (100 nm particles), 0.01% v/v (250 nm particles), 0.02% v/v (500 nm particles), 100 mg/mL (PEG 400), 10 mg/mL (PEG 6 kDa), 0.5 mg/mL (PEG 200 kDa), 10 mg/mL (pullulan), 10 mg/mL (dextrin), 0.25 mg/mL (fluorescent PEG 5 kDa). The acquisition time was 5 s, and 10-20 acquisitions were taken for each sample. The 1 μm and 5 μm microparticles were characterized using optical microscopy.

### Example 2

### Exemplary Polymers for Hydrogel Compression.

The following polymers were used to compress mucus hydrogel (see Examples 8 to 11), (all in 1× PBS): PEG 400, weight-averaged molecular weight 380-420 Da (Acros Organics, Pittsburgh, Pa., USA); PEG 6 k, weight-averaged molecular weight 5.6-6.6 kDa (Acros Organics); PEG 200 k, viscosity-averaged molecular weight 200 kDa (Sigma-Aldrich); Dextrin, average molecular weight between ˜1-70 kDa (40-43) (Walgreens, Deerfield, Ill., USA); Pullulan from Aureobasidium pullulans, average molecular weight between ˜50 kDa-4 MDa (44-48) (Sigma-Aldrich); Pectin from apple, weight averaged molecular weight ˜100 kDa (49) (Sigma-Aldrich).

### Example 3

### Animal Model for Hydrogel Detection

Except where otherwise noted, all mice were male or female specific pathogen free (SPF) or germ-free (GF) C57BL/6 mice between 2-6 months of age, fed a standard solid chow diet and given water ad libitum. The GF chow was autoclaved and was formulated to have similar nutritional profile after autoclaving as the SPF chow.

The mice given only sucrose or only sucrose+PEG were first raised on a standard solid chow diet and given water ad libitum, then maintained on a restricted diet consisting only of 5% sucrose or 5% sucrose+7% PEG 200 k in 1× phosphate buffered saline (PBS, pH 7.4, without calcium and magnesium, Corning, Corning, NY, USA) given ad libitum for the 24 h period preceding euthanasia. Four hours after administering each of the restricted liquid diets, each test mouse was moved to a new, clean cage to minimize the effects of coprophagy.

### Example 4

### Hydrogel Mucus Imaging

All imaging was performed using a Zeiss LSM 510 upright confocal microscope, using either confocal fluorescence microscopy (543 nm excitation/560 nm long-pass filter, or 488 nm excitation/505 nm long-pass filter), confocal reflectance microscopy (514 nm excitation/505 nm long-pass filter), or two-photon microscopy (800 nm excitation/650 nm long-pass filter). 3D stacks consisting of multiple xy slices at different z positions were collected.

### Example 5

### Hydrogel Thickness Detection in Unwashed Tissue

Each mouse was euthanized, the colon was removed and immediately flushed gently with Fluorinert FC 40 oil (3M, St. Paul, Minn., USA), which is immiscible with the aqueous contents of the colon. The colon segment was then immediately cut open along the longitudinal axis, and the opened tissue (luminal surface facing upward) was mounted onto a glass slide or a Petri dish using GLUture topical tissue adhesive (Abbott, Abbott Park, Ill., USA). ˜0.5-2 mL of additional FC 40 oil was then gently deposited onto the exposed luminal surface.

The FC 40 is immiscible with water and with the mucus hydrogel; this procedure thus retained the adherent mucus in its in vivo “unwashed” state and prevented it from dehydrating. The explant was imaged with two-photon microscopy. For some mice, multiple explant samples were taken and for some explant samples, multiple 3D stacks at different fields of view were collected. The hydrogel thickness was detected through imaging of the unwashed tissue.

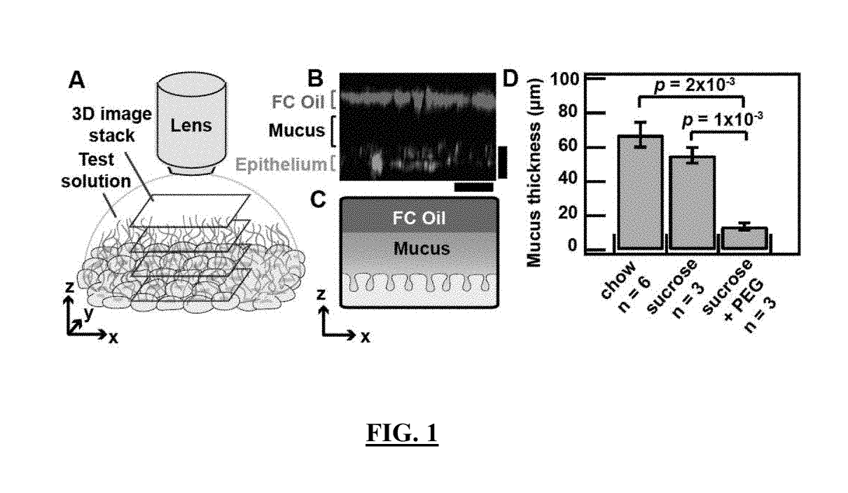

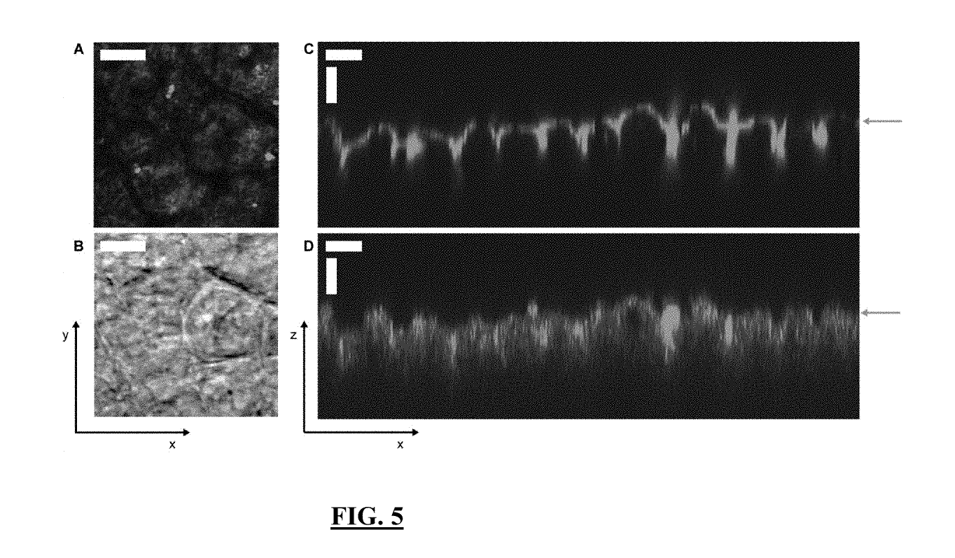

The mean mucus thickness (grey bars in FIG. 1 and FIG. 4) for each stack obtained from an explant was determined by measuring the distance between the epithelial surface (FIG. 5; Panel A-B) and the FC oil-hydrogel interface at five random positions in xy. In some cases this was repeated for multiple fields of view. When multiple colonic explants were obtained from a single mouse, the mean mucus thickness of an individual mouse was calculated. In FIG. 1 and FIG. 4, the thickness values reported are the mean values of the individual mice thicknesses. The error bar on each value reported in FIG. 1 and FIG. 4 is the standard error of the mean (SEM), calculated by taking the standard deviation of mucus thickness for a single mouse and dividing by (n, number of different mice).

### Example 6

### Hydrogel Thickness Detection in Washed Tissue

Each mouse was euthanized; the colon was removed and immediately flushed gently with ice-cold 1× PBS, and placed ˜1 cm long segments of the mid-colon in ice-cold PBS. The colon segments were then cut and mounted as described for unwashed tissues, always ensuring the explant surface was covered in PBS to prevent any dehydration or ionic imbalance, and surrounding (but not contacting) the tissue with >10˜10 μL drops of water to maintain a humid environment. The hydrogel thickness was detected through imaging of the washed tissue.

The measured mucus hydrogel thickness was consistent with the distance measured when imaged using FC oil and consistent with other reported measurements (3), did not change appreciably over an observation time of 2.5 h, and was similar for probes of other sizes (250 nm in diameter or larger), further confirming the validity of the approach. An additional ˜10-200 μL drop of test solution containing the fluorescent probes was then gently deposited onto the explant. The explant was imaged with confocal reflectance or two-photon microscopy.

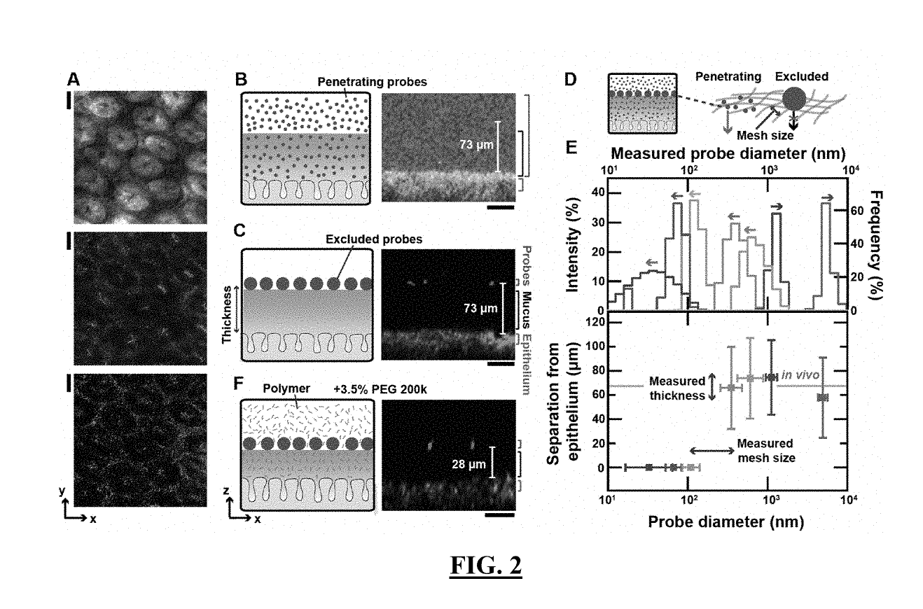

For some mice, multiple explant samples were taken and for some explant samples, multiple 3D stacks at different fields of view were collected. The levels of the images in FIG. 2, Panel A were non-linearly adjusted in Adobe Illustrator for clarity in print using the following input and output levels: 82, 1, 246/0, 255 (bright field), 34, 0.78, 172/0, 205 (confocal reflectance), 51, 0.91, 140/0, 255 (two-photon).

### Example 7

### Thickness Measurements of Washed Mucus Hydrogel

In each experiment, after placing a suspension of 1 μm diameter microparticles onto the exposed luminal surface, the tissue was incubated at 4° C. for 1-2 h, longer than the time required for the microparticles to diffuse across the vertical extent of the mucus in free solution (40 min). This ensured that the microparticles deposited onto the mucus hydrogel surface. both the epithelium and the deposited microparticles were simultaneously imaged using confocal or two-photon reflectance microscopy.

To determine the mean mucus thickness for tissue obtained from a single mouse (green, light blue, dark blue and pink points in the bottom graph of FIG. 2, Panel D), for each stack on a washed explant, the distance between the epithelial surface and the center of the deposited microparticles was measured at five random positions in xy spanning the entire field of view. In some cases this process was repeated for multiple fields of view. If multiple colonic explants were obtained from the same mouse, the thickness was measured in the same way. Each of these individual thickness measurements at each xy position from all the individual mice explants and fields of view was then taken, and the mean and standard deviation were calculated. The thickness values reported in FIG. 2, Panel D are these mean values, and the error bars are the associated standard deviation. The washed values and error bars reported FIG. 4 (purple bars), were determined as described in Example 5.

### Example 8

### Quantitative Detection of Polymer-Induced Compression of Washed Mucus Hydrogel

After measuring the initial washed mucus thickness, ˜0.2-2mL of the test polymer solution was gently deposited onto the exposed luminal surface and then collected the same 3D stacks at the same xy fields of view

To measure the “percent compression”, or the overall percentage change in the thickness, of the colonic mucus after exposure to the polymer solution, the thickness was measured before and after exposure to the solution at the same five xy positions, using the distance between the epithelial surface and the deposited microparticles in the 3D stacks.

To calculate the percentage compression, the percentage change in the thickness measured was calculated, as well as the measurement uncertainty (using the optical slice thickness as the experimental uncertainty in the measured thickness), at each of these five xy positions. The percentage compression was calculated as the mean of these five measured values. The error bars show the uncertainty in the percentage compression measurement, which was calculated using the experimental uncertainty in each of the five strain measurements.

### Example 9

### In Vivo Detection of the Thickness of Colonic Mucus Hydrogel

To probe the in vivo thickness of murine colonic mucus, a label-free technique was developed that eliminates evaporation and avoids the use of any washing, fixative, labeling, or dehydrating agents that could alter mucus structure, as described in the above examples.

Freshly-excised colon explants obtained from mice at least 8 weeks old were used—whose mucus hydrogel has been found to be fully-developed and stable (19). The luminal contents were gently removed using FC-40 oil, a fluorocarbon fluid that is immiscible with, and denser than, water. Each explant was opened along the intestinal axis and mounted flat, with its luminal surface facing upward and coated with FC oil. An upright confocal microscope equipped with a dry objective lens was then used to image, in three dimensions, the exposed epithelial surface and the oil overlying the adherent mucus hydrogel (FIG. 1, Panel A).

Both the epithelial surface (FIG. 5, Panel A-B) and the oil-mucus interface were first identified using confocal reflectance microscopy (FIG. 1, Panel B-C). The distance between the two provided a measure of the mucus hydrogel thickness. A comparable mucus thickness of 67±7 μm or 55±5 μm (mean±SEM, n=6 or 3, P=0.3) were measured for control mice fed a standard chow diet or a sucrose solution (FIG. 1, Panel D), consistent with previous measurements (3).

To investigate the role of polymers in altering mucus structure, mice was then fed with the same sucrose solution, with added polyethylene glycol (PEG), an uncharged polymer that is well-characterized, is often used as a therapeutic in the gut (8, 9), and has minimal chemical interactions with biomolecules (20). PEG of an average molecular weight ˜200 kDa was used, denoted as PEG 200 k. Unexpectedly, the mucus hydrogel was significantly thinner for these mice, 14±2 μm (mean±SEM, n=6, P=2×10⁴; FIG. 1, Panel D). This finding demonstrates that such polymers can in fact alter the structure of mucus.

### Example 10

### Ex Vivo Characterization of Colonic Mucus Hydrogel and Related Polymer Compression

To better understand hydrogel compression by polymers, the imaging approach was modified so the mucus hydrogel ex vivo can be directly imaged while the physicochemical composition of the aqueous solution to which mucus is exposed can be simultaneously controlled. Freshly-excised murine colon explants were used, and then cut open along the intestinal axis and mounted flat. Instead of using FC oil as the test solution, the luminal contents were cleared and the luminal surface was coated with cold saline to remove soluble components, including any polymers. A water-immersion objective lens was used to identify the epithelial surface (FIG. 2, Panel A) and corroborated with lectin staining (FIG. 5, Panel C-D).

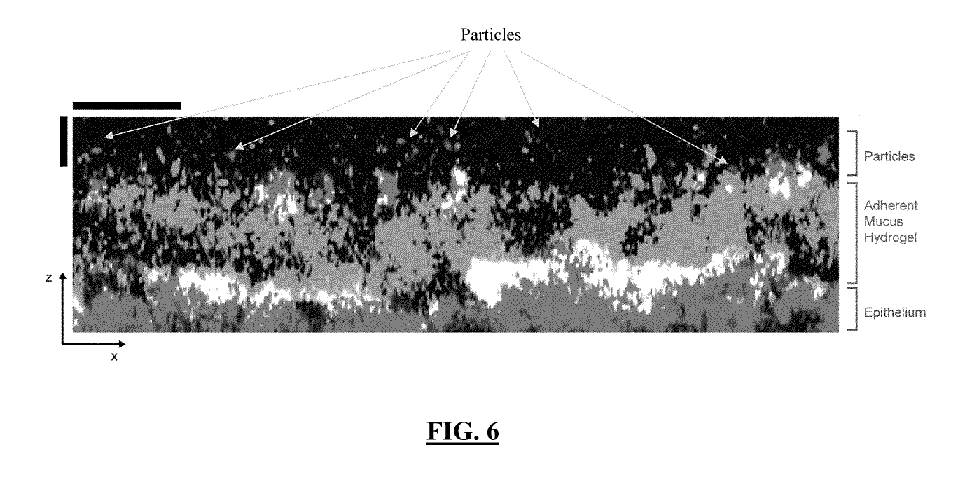

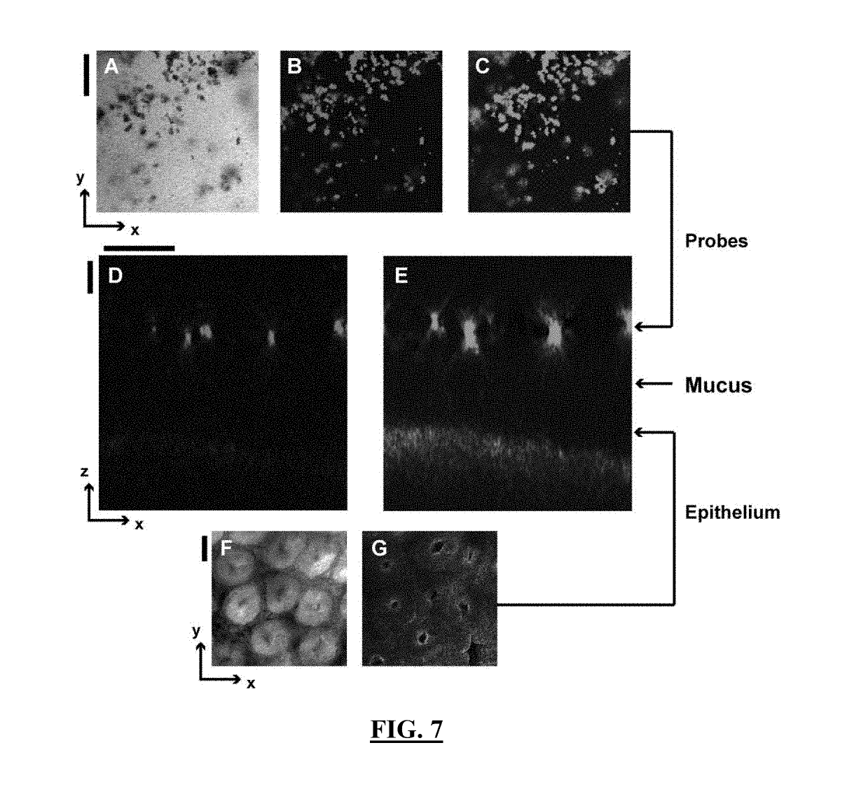

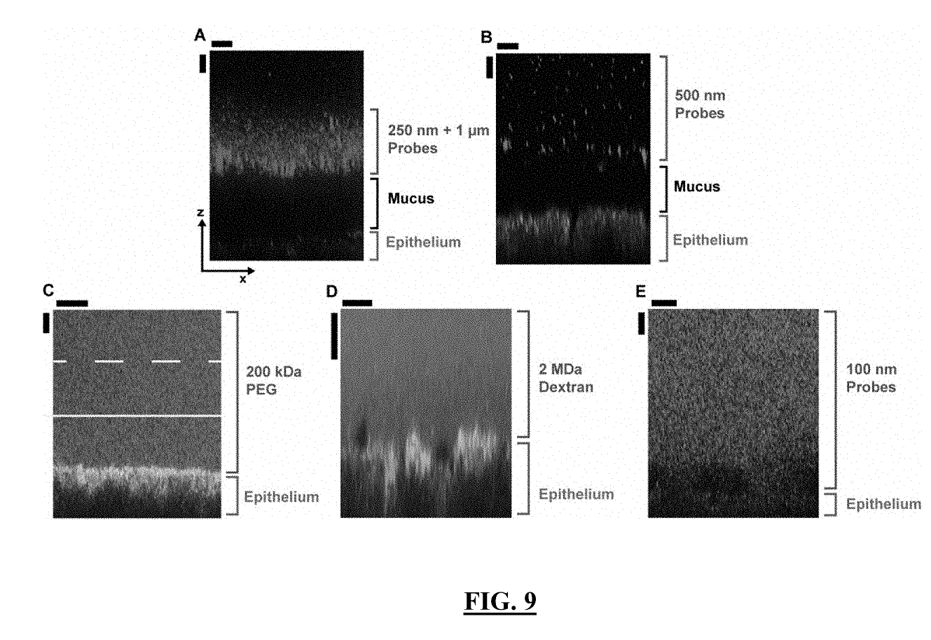

To identify the luminal surface of the mucus hydrogel, a solution of 1 μm diameter microparticle probes was deposited onto the explant surface. These probes did not penetrate, but instead settled on top of, the mucus hydrogel, indicating that they were larger than its mesh size (FIG. 2, Panel C). Previous studies have validated that this region of probe exclusion corresponds to the adherent mucus hydrogel (14, 18, 21, 22); it was further confirmed using lectin staining (FIG. 6). Measuring the distance between the excluded probes (FIG. 7) and the underlying epithelial surface thus provided a measure of the mucus thickness, 75±30 μm (mean±SD), consistent with the distance measured when imaged using FC oil and consistent with other reported measurements (3). Hydrogel thickness did not change appreciably over an observation time of 2.5 h. similar results were also found using probes of other sizes (FIG. 9, Panel A-B): all probes 250 nm in diameter or larger were excluded from the mucus, and yielded comparable mucus thickness values (FIG. 2, Panel E). By contrast, probes 100 nm in diameter or smaller (FIG. 9, Panel C-D) penetrated the mucus and reached the underlying epithelium, indicating that they were smaller than the mesh size (FIG. 2, Panel B and E). It is concluded that the mesh size of the adherent mucus hydrogel was between 100-250 nm, in good agreement with measurements of the mesh size of other mucus hydrogels (23, 24).

Having established a method for characterizing mucus hydrogel structure ex vivo, the influence of polymers was tested next. A solution of the same PEG was placed onto the explant surface, and the mucus hydrogel thickness was continually monitored using the deposited microparticles.

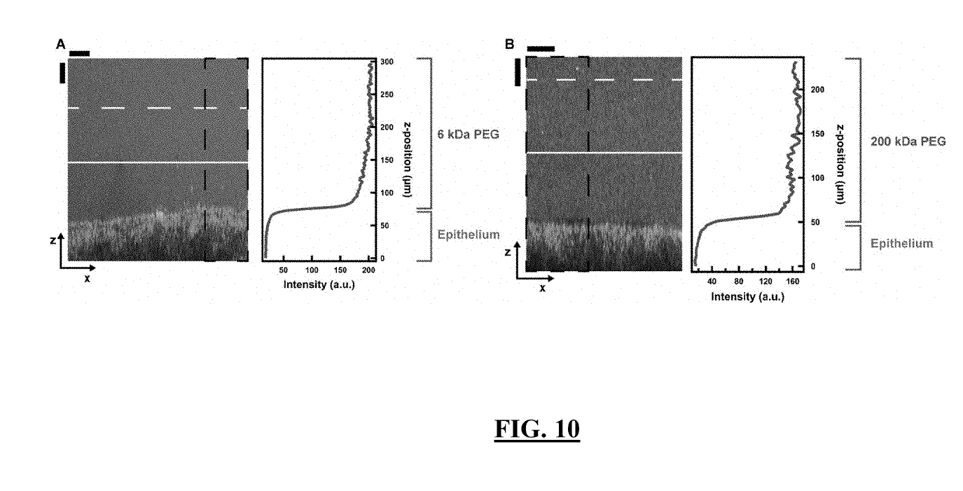

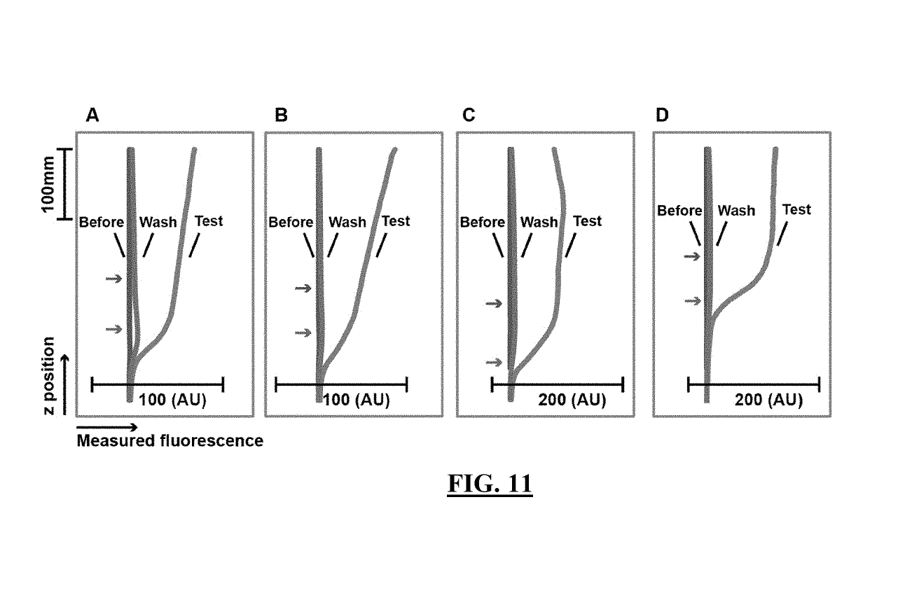

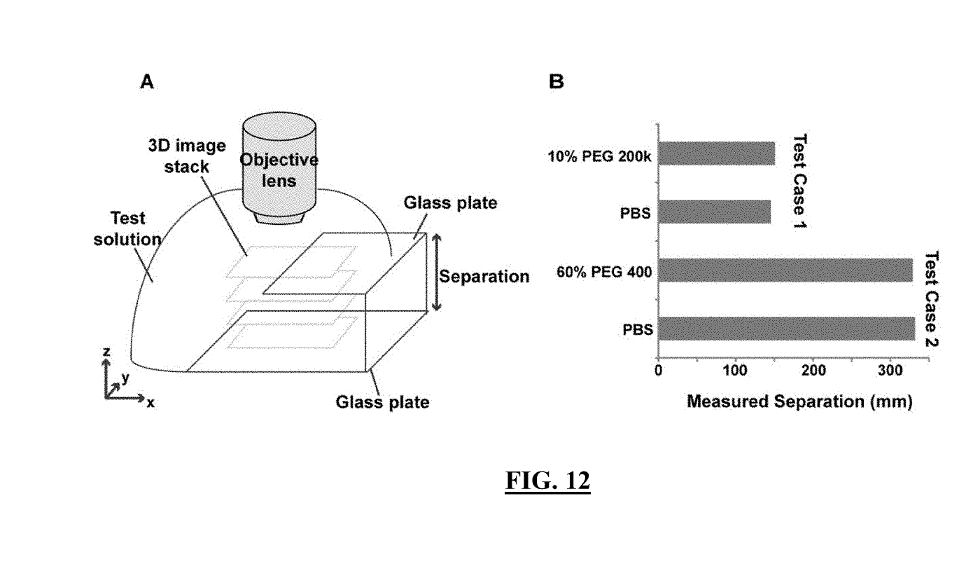

The PEG penetrated the mucus and reached the underlying epithelium (FIG. 10) and this penetration was reversible, suggesting that strong PEG-mucus chemical interactions—such as complexation, which can play a role under different conditions than those explored here were absent (FIG. 11). Nevertheless, the mucus hydrogel compressed by approximately 50-60% of its initial thickness within ˜5-20 min (FIG. 2, Panel E), and the level of compression appeared to be stable over an observation time of at least ˜100 min. It was verified that any optical effects induced by the polymer solution did not appreciably affect the z measurements (FIG. 12). Interestingly, compression was at least partly reversible; the mucus hydrogel re-expanded to approximately 90% of its original thickness after PEG was removed by washing the explant. These findings suggest that the polymer-induced compression observed in the FC oil experiments can be reproduced and investigated further ex vivo.

### Example 11

### Experiments Using Liquid Fraction of Colonic Contents

SPF and GF mice were fed ad libitum on either a standard chow diet, 5% w/v sucrose in 1× PBS, or 5% w/v sucrose with 7% w/v PEG 200 k in 1× PBS

Immediately after euthanizing a mouse, its colonic contents was collected in a polypropylene spin column with a 30 μm pore size filter (Thermo Scientific Pierce, Waltham, MA, USA), always kept on ice, and centrifuged at 17,000 g for 100 min at 4° C. The liquid supernatant from the collection tube was then collected. The liquid fraction thus obtained from multiple mice, both male and female, 3-4 months in age, was combined to obtain enough sample for the experiments, and stored aliquots at −20° C. until experimental use.

For each of the experiments shown in FIG. 4, Panel C; a washed explant was incubated with 1 μm microparticles and used two-photon microscopy to first measure the initial, washed mucus thickness.

The frozen liquid fraction of colonic contents was then thawed, 100 μL of it was gently deposited on the exposed luminal explant surface, and re-imaged to measure the change in mucus thickness. Successive 3D stacks were then obtained to verify that the thickness did not change in time over a time period of ˜10-30 minutes. Multiple 3D stacks were collected at different fields of view on the same tissue explant, and for different tissue explants obtained from multiple mice.

The difference between the SPF and GF chromatograms in FIG. 14, Panels A-B suggested that, as expected (94-98), the GF contents were enriched in polymers of higher molecular weight compared to the SPF contents, and [1-5] that these polymers were comparable in size to ˜200-700 kDa pullulan standards.

It was found that the SPF contents did not appreciably compress colonic mucus, indicating that any residual polymers present in the SPF contents (after microbial degradation) were insufficient to compress the hydrogel; this result is also consistent with the observation that SPF mice and mice maintained on a sucrose diet had colonic mucus hydrogels of comparable thickness (FIG. 1, P=0.3). By contrast, it was found that the GF contents compressed colonic mucus by ≈70% of its initial washed thickness, for washed explants obtained from either SPF or GF mice (FIG. 4, Panel C). This finding indicates that gut microbes, by modifying the polymeric composition of intestinal contents, can actively modulate the compression state of the colonic mucus hydrogel (FIG. 4, Panel D).

### Example 12

### Flory-Huggins Polymer Induced Compression of a Hydrogel Model

Control of the volume mucus hydrogel by polymers such as PEG can be described according to the Flory-Huggins theory of polymer solutions as exemplified below. This is because adherent mucus is a hydrogel with a network (4, 36, 50) comprised of MUC2 proteins having alternating hydrophilic, densely-glycosylated regions, which make up the strands of the hydrogel network, and hydrophobic, non-glycosylated regions, which help to cross-link the network, which is also cross-linked via physical entanglements and disulfide bonds (51, 52).

The mucus was therefore modeled as a cross-linked hydrogel swollen in a good solvent. For simplicity, this hydrogel was treated as being structurally isotropic; the model does not incorporate any possible supramolecular structuring of the colonic mucus hydrogel (36). An assumption was made that the mucus behaves as an elastic gel; while hydrogels, including colonic mucus, are known to be viscoelastic—they relax stresses over long times—the reversibility of the observed polymer-induced compression suggests that the colonic mucus is elastic on the timescale of the experiments. Moreover, this assumption has been successfully used to describe the compression of synthetic hydrogels (29, 30).

First, the total free energy of the ternary solvent-mucus-polymer system, G, was calculated given by the sum of the elastic free energy, Gₑₗ—which accounts for deformations of the individual mucus network strands, thus inhibiting the unphysical case of full mixing of the mucins and solvent—and the free energy of mixing the polymer and the solvent with the mucus hydrogel, Gₘ. The buffered aqueous solutions are characterized by a Debye screening length 0.7 nm, over two orders of magnitude smaller than the hydrogel mesh size; therefore electrostatic effects were not consider (53-57). The total change in free energy can thus be written as

ΔG=ΔGₘ+ΔGₑₗ (Eq. 11)

and ΔGₘ is given by the Flory-Huggins (29, 37, 38) free energy of mixing,

$$Δ {G}_{m} = RT ( {\sum }_{i} {n}_{i} ln {v}_{i} + {\sum }_{i < j} {n}_{i} {v}_{j} {χ}_{ij} ) \qquad ( Eq . 12 )$$

where R is the gas constant, T is the temperature, nᵢ is the number of moles of species i, vᵢ is the volume fraction of species i, and χᵢⱼ is the Flory-Huggins interaction parameter between species i and j; here, solvent, mucus and free polymers are denoted as i=S, M, P, respectively. To describe the free energy of elastic deformation, rubber elasticity was used, assuming affine deformation of the network (29, 37):

$$Δ {G}_{el} = \frac{3}{2} \frac{RT}{{N}_{M} {V}_{S}} [ {( \frac{{v}_{M}^{0}}{{v}_{M}} )}^{2 / 3} - 1 - {ln ( \frac{{v}_{M}^{0}}{{v}_{M}} )}^{1 / 3} ] \qquad ( Eq . 13 )$$

where v_{M} is the mucus hydrogel volume fraction, v_{M}⁰ is the mucus hydrogel volume fraction in its initial preparation state, V_{S} is the molar volume of the solvent, and N_{M} is the average number of mucin Kuhn segments, the stiff segments making up each mucin network strand, between cross-links of the network. More sophisticated forms of the elastic free energy would be interesting to explore in future work; it is noted that the exact choice of the elastic energy may not impact the calculated hydrogel compression trends considerably (28, 29).

At equilibrium, the chemical potentials of both the solvent and the free polymer, μ_{S}=∂G/∂n_{S} and μ_{P}≡∂G/∂n_{P}, must be equal inside and outside of the mucus network:

μ_{S}ⁱⁿ=μ_{S}^{out} (Eq. 14)

μ_{P}ⁱⁿ=μ_{P}^{out} (Eq. 15)

wherein n_{P} and n_{S} are the respective numbers of moles.

By substituting equations 2 and 3 into equation 1, and differentiating with respect to the number moles of solvent and free polymer, equations 1-4 representing the central result of the Flory-Huggins model are obtained and have been successfully used to describe polymer-induced compression of synthetic hydrogels (29). These equations are also subject to the constraints v_{S}ⁱⁿ+v_{M}+v_{P}ⁱⁿ=1 and v_{S}^{out}+φ=1.

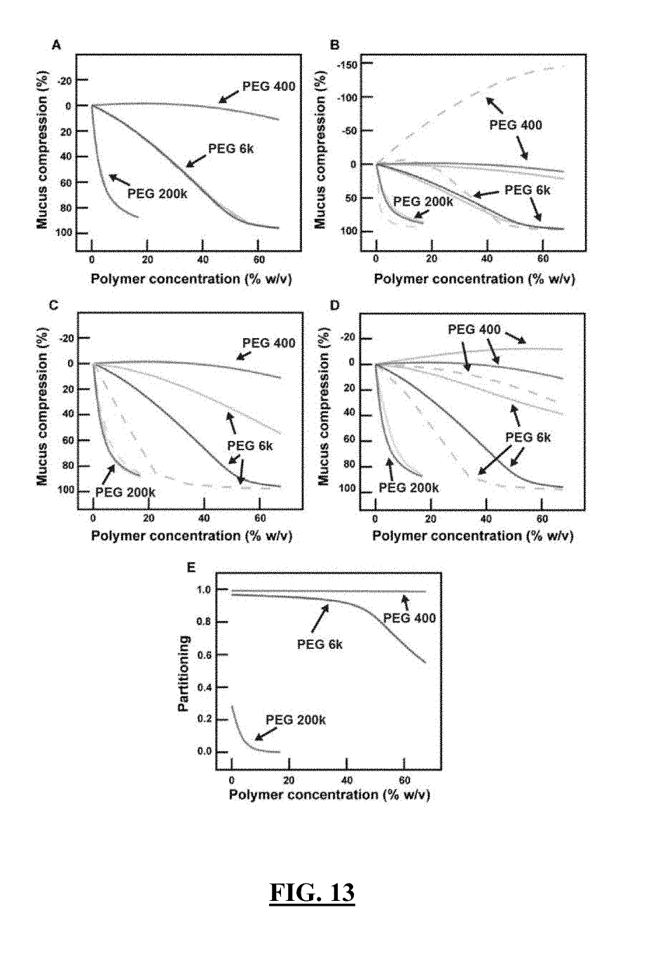

Firstly the polymer-free case (φ=0) was treated, which describes the initial swollen state of the mucus hydrogel. The system is described by Eq. 1 with μ_{S}ⁱⁿ=μ_{S}^{out}=0 and v_{P}ⁱⁿ=0; this provided us with a relationship between v_{M}⁰, χ_{SM}, N_{M}, and the mucus volume fraction in this initial swollen state, which is denoted as v_{M}^{s}. Direct measurements of v_{M}^{s} are lacking; a value of v_{M}^{s}=0.01 is chosen, well within in the range of estimates (58-62) of the volume fraction of swollen mucus, and tested the sensitivity of our results to variations in the numerical parameters used, with the constraint relating v_{M}⁰, χ_{SM}, N_{M}, and v_{M}^{s} (FIG. 13).

As a simplifying assumption, v_{M}⁰ was taken to be approximately equal to the mucin volume fraction when initially packed in secretory granules, before being released into the intestinal lumen to form the swollen, cross-linked adherent hydrogel. It is noted that the packed mucus within the granules is condensed by high concentrations of Ca²⁺ which, upon mucus expulsion, is likely diluted away; however, it is speculated that many of the crosslinks formed within the granules via other interactions (e.g. physical entanglements and disulfide bonds) can remain, due to the close physical proximity of the mucins to each other.

Moreover, it was found in the sensitivity analysis (FIG. 13) that the results are only weakly sensitive to the value of v_{M}⁰. A value v_{M}⁰=0.13 was therefore chosen, within the range of published measurements (63-65) for mucin and other similar secretory granules. Water is expected to be a good solvent for the mucin network strands, due to the preponderance of hydroxyl, carboxyl and sulfate groups in the glycosylated domains; χ_{SM}=0 was therefore chosen. N_{M} was estimated using published measurements in two different ways. In the first approach, measured values (58, 66-69) of the MUC2 radius of gyration, R_{g,M} , and Kuhn length, b_{M}, were used and combined with the relationship for mucus strands swollen in a good solvent (38, 61, 70, 71), R_{g,M}≈b_{M}N_{M}^{3/5}. In the second approach, the direct measurements of the mucus hydrogel mesh size, combined with the published measurements of b_{M}, was used to estimate N_{M}. In both cases, it was found N_{M}≈20-10,000. The values of v_{M}⁰, χ_{SM}, and v_{M}^{s}, together with Eq. 1, yielded N_{M}≈1000, in this estimated range; N_{M}=1000 was therefore chosen. Again, qualitatively similar results for different values of N_{M} were found (FIG. 13).

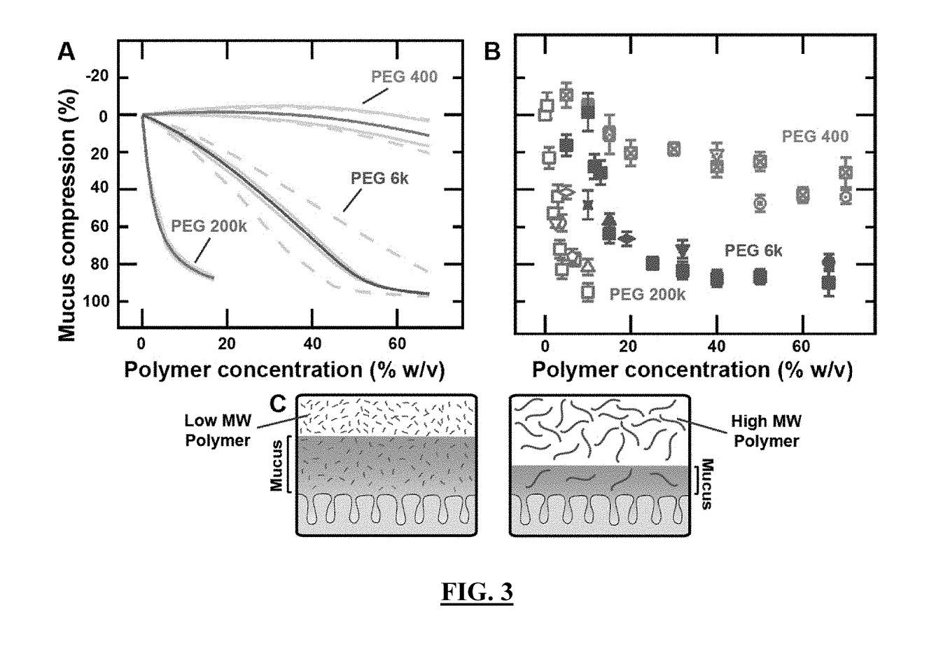

Next, how added polymer (φ>0) changed the extent to which the mucus hydrogel is swollen, and therefore, its equilibrium thickness, was investigated, Eqs. 4-5 were numerically solved for v_{M} and v_{P}^{m}, varying φ; this yielded the curves presented in FIG. 3, Panel A. The cases where the added polymer is PEG 400, 6 k, or 200 k, as used in the experiments were focused. The number of segments of each PEG, y, was taken to be the number of PEG Kuhn segments, and estimated (38) using the relationship R_{g,P}≈b_{P}y^{α}, where R_{g,P} and bₚ are the PEG radius of gyration and Kuhn length, respectively, by choosing α=0.58, consistent with the measured range (38, 72-76) α=0.537-0.588. Published measurements (77-79) yield b_{P}≈0.76-1.8 nm; b_{P}=1.28 nm was therefore chosen, in this range. R_{g,P} was estimated using the measurements of the PEG 400, 6 k, and 200 k hydrodynamic radii, and converted to radii of gyration using the Kirkwood-Riseman relationship (80-82). The relationship between R_{g,P}, b_{P}, and y thus yielded y=1, 4, and 146 for PEG 400, 6 k, and 200 k, respectively, which were used for the main simulations (FIG. 3, Panel A). Based on published measurements for PEG (29, 83), χ_{SP}=0.45 was set. The chemical interactions between PEG and mucins are thought to be slightly attractive or neutral. χ_{MP} was therefore estimated to be between 0 and 0.5, and chose χ_{MP}=0.3.

This Flory-Huggins framework has been successfully applied to qualitatively describe polymer-induced compression of a number of synthetic hydrogels (29, 30, 84-88). It is a simple mean-field theory, does not take into account correlations between monomers, and assumes affine deformation of a homogeneous gel. However, similar behavior between the two, using parameters that are consistent with experimentally measured values, were observed. In particular, the Flory-Huggins calculations showed that the free polymer does induce compression of the network, even though in the calculations the polymer could penetrate into the mucus hydrogel, and the trends observed experimentally are qualitatively similar to those predicted by the model.

Moreover, it was found that polymers of higher molecular weights required a lower monomer volume fraction to compress the network consistent with our experimental observations. One reason for this is the entropic penalty paid by PEG to penetrate the mucus; because this penalty is larger for larger polymers, they are more likely to be excluded from the mucus hydrogel, and therefore can compress it more by elevating the difference between external and internal osmotic pressure. Consistent with this expectation, it was found that the higher molecular weight PEG was more likely to be excluded from the mucus hydrogel (FIG. 13, Panel E).

More sophisticated modeling could build on the work presented here by incorporating effects such as structuring of the colonic mucus hydrogel (36), viscoelastic relaxation of the mucus network, chemical adhesion (39) or electrostatic interactions, or polymer complex formation. For example, PEG has been observed to form complexes with polycarboxylic acids (29, 89-93), via hydrogen bonding between the ether oxygen of PEG and un-dissociated carboxylic groups; similar effects could play a role in our experimental system. It is noted, however, that at the physiological pH explored in our work, the carboxyl groups found on the sialic acid residues of mucins are negatively charged (50, 52) and complexation is unlikely (FIG. 11).

### Example 13

### Flory-Huggins Polymer-Induced Compression of Colonic Mucous Hydrogel

Large non-penetrating polymers have been used to osmotically compress synthetic hydrogels (25) and even the periciliary brush after mucus removal in the mammalian lung (26). However, the possibility that even polymers small enough to penetrate a hydrogel could compress it was first recognized by Brochard in 1981 (27), and was subsequently investigated both theoretically and experimentally (28, 29). In this case, hydrogel compression arises from a combination of enthalpic and entropic effects. For example, the polymers can reduce the effective solvent quality of the hydrogel environment, due to enthalpic interactions with the hydrogel network strands, forcing the hydrogel to reduce its hydrated volume and compress.

Another effect arises from the free energy penalty associated with penetrating the hydrogel mesh: this can lead to an elevated polymer concentration, and therefore, an elevated osmotic pressure, outside the hydrogel, which similarly forces the hydrogel to compress. Clarifying the role of these, and other, different effects remains unresolved, even for the case of synthetic hydrogels; however, such effects can be described collectively using the classic Flory-Huggins theory of polymer solutions (28, 29). We therefore asked whether this physical framework could also describe polymer-induced compression of the colonic mucus hydrogel. Indeed, while the predictions of this theory have been experimentally verified using a few model synthetic hydrogels (29, 30), its applicability to the more complex case of biological hydrogels like colonic mucus is unclear. One signature of this form of compression is its tunability: more concentrated polymer solutions should induce more hydrogel compression (28, 29). Consistent with this prediction, it was found that mucus compression was tunable by PEG concentration (green points, FIG. 3, Panel B).

To test the applicability of Flory-Huggins theory, the same theoretical framework (29) was used to describe the experimental system. The mucus was first modeled as a swollen, cross-linked, hydrogel. Then, how the addition of polymers changes the extent to which the mucus hydrogel is swollen and its equilibrium thickness was considered. Simplifying assumption (29, 30) was made that the mucus behaves as an elastic gel on the timescale of our experiments, even though hydrogels, including colonic mucus, are known to be viscoelastic—they relax stresses over long times. This assumption is supported by the observations that the hydrogel thickness remained stable in either the uncompressed or polymer-induced compressed states (over observation times of at least ˜100 min). It is further supported by the reversibility of the observed compression. The total free energy of the ternary solvent-mucus-polymer system, G, was then calculated as the sum of the elastic free energy, which accounts for deformations of the individual mucus network strands, and the free energy of mixing the polymer and the solvent with the mucus hydrogel. This total free energy was then used to calculate the chemical potentials of both the added PEG and the solvent, μ_{P}≡∂G/∂n_{P} and μ_{S}≡∂G/∂n_{S}, respectively, both inside and outside of the mucus network; n_{P} and n_{S} are the respective numbers of moles.

At thermodynamic equilibrium, μ_{S}ⁱⁿ=μ_{S}^{out} and μ_{P}ⁱⁿ=μ_{P}^{out}; these equalities enabled to numerically calculate the equilibrium mucus thickness for a given PEG concentration (details of calculations, parameters used, and sensitivity to parameters are described in the previous examples). Consistent with the experimental observations, the Flory-Huggins model predicted that exposure to PEG compresses the adherent mucus hydrogel. Moreover, the model predicted (green curve, FIG. 3, Panel A) a similar dependence of mucus compression on PEG concentration as measured in the experiments using microparticles (green points, FIG. 3, Panel B).

Another key prediction of the model is that the extent of mucus compression should depend on the polymer molecular weight: for a given PEG concentration, smaller polymers should compress the mucus hydrogel less (FIG. 3, Panel A). One intuitive explanation for this is the free energy penalty paid by PEG to penetrate the mucus, which is smaller for smaller polymers; thus, even though they can exert a larger osmotic pressure, smaller polymers are less likely to be excluded from the mucus hydrogel (FIG. 13, Panel E), and are expected to compress it less (FIG. 3, Panel C). To test this prediction, the extent of mucus compression induced by two smaller polymers, PEG 6 k and PEG 400, was measured. These polymers again compressed the mucus hydrogel within 5 min, and the compression level appeared to be stable over an observation time of up to several hours.

Despite the mean-field nature of the Flory-Huggins model, which is not expected to capture the full complexity of the experiments, qualitative similarities between the calculations (FIG. 3, Panel A) and the experimental data (FIG. 3, Panel B) were observed. Similar results were also found for varying values of the model parameters (FIG. 3, Panel A, FIG. 13, Panel A-D). Moreover, the observed compression was similar for mice of different genders and strains, for washed explants originating from germ-free or microbe-colonized mice, for different buffers, in the presence and the absence of Mg²⁺ ions, for buffers also containing protease inhibitor, for experiments performed at 22° C. or 37° C., and for a similar, but charged, polymer, demonstrating that the results were not an artifact of the choice of the animal model or details of experimental conditions. The similarity between the theoretical predictions and the experimental data suggests that Flory-Huggins theory provides a physical description of the concentration and molecular weight-dependence of the polymer-induced compression of colonic mucus, and provides a foundation for more sophisticated modeling to better characterize the full complexity of this phenomenon.

### Example 14

### Microbes Can Modulate Mucus Compression

Given the diversity of polymers abundant in fruits, vegetables, and food additives, dietary polymers were tested next whether they also compress colonic mucus. Three common dietary polymers, dextrin, pectin and pullulan, were tested.

Exposure to each of these polymer solutions caused the colonic mucus hydrogel to compress in a concentration-dependent manner (FIG. 4, Panel A). Moreover, as with PEG, for a given polymer concentration, the larger polymers, pectin and pullulan, compressed the mucus more than the smaller polymer, dextrin. These observations demonstrate that, similar to the case of PEG, dietary polymers present in the gut can also induce mucus compression in a manner that depends on the physical properties of the polymers themselves.

Given the results indicating that mucus compression can depend on the polymer molecular weight, it was hypothesized that microbial degradation of polymers into smaller fragments (6, 7) may actively modulate compression in vivo. Indeed, it was found that while pectin strongly compressed the colonic mucus hydrogel (FIG. 4, Panel A, blue points), a small molecule, acetate—a typical product of pectin degradation and fermentation by gut microbes—did not (500 mM acetate compressed the mucus only by ≈10%). Moreover, using the wash-free FC oil methodology as in FIG. 1, it was found that the adherent mucus of germ-free (GF) mice was only ≈25% as thick as that of specific-pathogen-free (SPF) mice in vivo (FIG. 4, Panel B), consistent with previous observations (3, 31). Thicker SPF mucus was previously attributed solely to altered mucus secretion by the host in response to the presence of microbes, and not to the difference in polymeric composition of the gut fluid.

Given the results, however, it was hypothesized that mucus compression by intestinal polymers may also contribute to this phenomenon: these polymers remain intact in GF mice, which lack the gut microbiota that normally degrade these polymers into smaller non-compressing fragments. In agreement with this hypothesis, washing the GF explant with excess cold saline, which should dilute out any polymers present in the sample, restored the mucus to the thickness observed in SPF mice (FIG. 4, Panel B). This result was surprising, because it could not have been the result of a host response to the presence of microbes.

To further test the effect of intestinal polymers on mucus compression, the liquid fractions of the colonic contents of GF and SPF mice were isolated and analyzed. As expected (FIG. 14), the GF contents were enriched in higher molecular weight polymers compared to the SPF contents[1-5], reflecting polymeric degradation by the SPF gut microbiota. It was therefore predicted that the GF contents would compress colonic mucus more than the SPF contents. In agreement with this prediction, while SPF contents did not appreciably compress colonic mucus, the GF contents compressed colonic mucus by ≈70% of its initial washed thickness, for washed explants obtained from either SPF or GF mice (FIG. 4, Panel C). This finding indicates that gut microbes, by modifying the polymeric composition of intestinal contents, can actively modulate the compression state of the colonic mucus hydrogel (FIG. 4, Panel D).

The data show that the extent of compression is strongly dependent on polymer concentration and molecular weight; this behavior is remarkably similar to the compression of synthetic hydrogels, which is known to arise from a combination of enthalpic and entropic effects. The role played by these different effects remains to be elucidated, even for the case of simple synthetic hydrogels. However, the data suggest that, similar to the synthetic case, polymer-induced compression of mucus—a complex biological hydrogel—can be described using Flory-Huggins theory. The results thus motivate further work studying the physics underlying hydrogel compression, and the theoretical description presented here provides a basis for more sophisticated biophysical modeling that could incorporate effects such as non-isotropic structure of the mucus network (36), viscoelastic relaxation of the mucus hydrogel, or electrostatic interactions. This could lead to new strategies for designing polymer-based therapeutics to controllably and predictably alter the morphology of gut mucus. Moreover, the examples herein described provide a general biophysical framework for investigating similar, previously overlooked, polymer-induced effects in other biological hydrogels, such as airway mucus, nasal mucus, cervico-vaginal mucus, or extracellular matrix in tissues.

### Example 15

### Polymeric Compositions are Subjected to Physiological Constraints

The osmotic pressure in the dilute regime is molecular weight dependent. Specifically for a given polymer concentration, the osmotic pressure can reduce for polymers of higher molecular weight. In some cases, the polymeric composition can consist of high molecular weight polymers chosen to have a target total osmotic pressure less than a physiological threshold of 0.74 MPa.

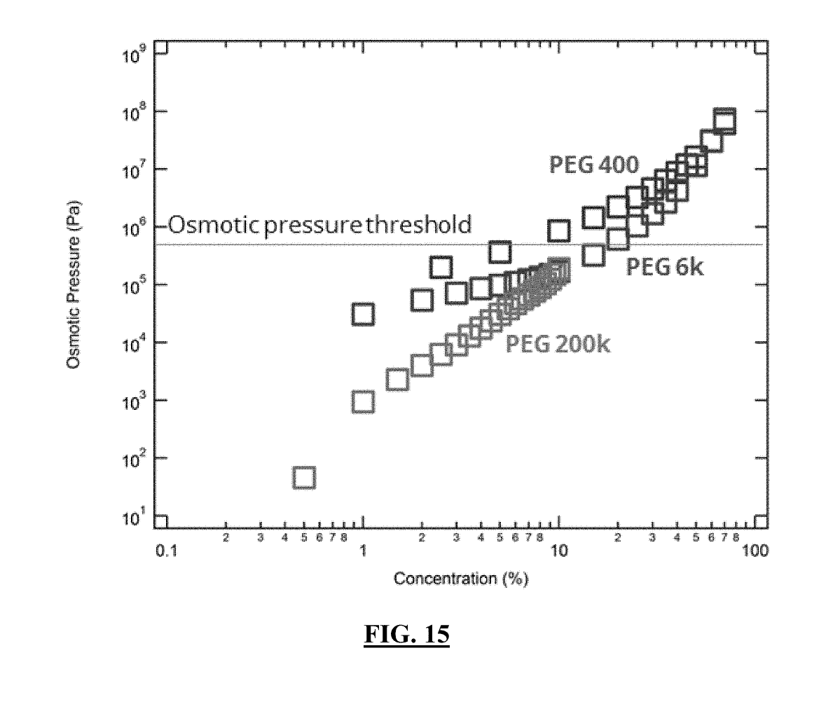

FIG. 15 shows published measurements of the osmotic pressure of PEG 400, PEG 6 k, and PEG 200 k solutions. Under the above discussed physiological threshold, PEG 400 can have a concentration lower than 7%, PEG 6 k can have a concentration lower than 20% and PEG 200 k can have a concentration lower than 20%.

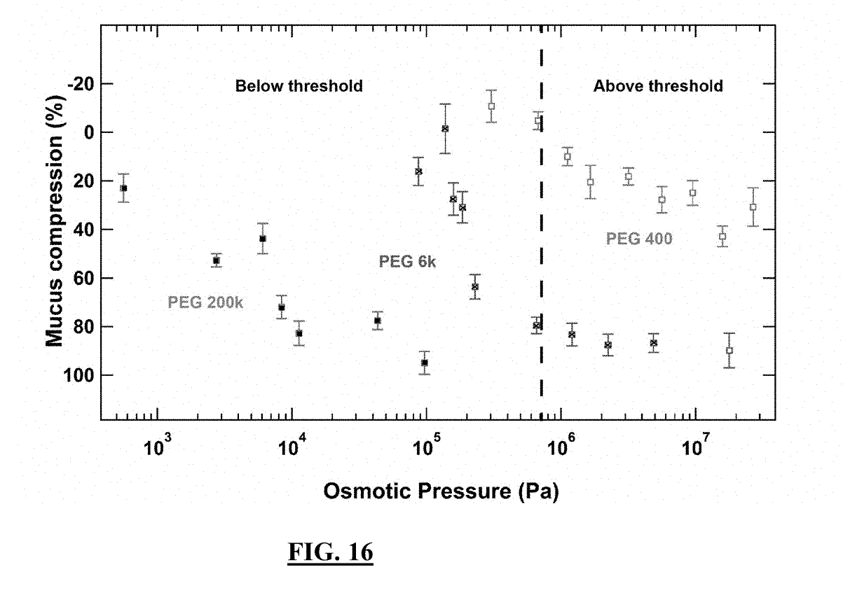

FIG. 16 shows experimental data for PEG 400, PEG 6000, and PEG 200 k, with the % compression as a function of the total osmotic pressure (from literature measurements) of each polymer solution. The data on the left side of the dashed line are below the 0.7 MPa threshold and the data on the right side are above the 0.7 MPa threshold.

As one demonstration of this phenomenon, a solution of 1.69% w/v PEO (polyethylene oxide) of average molecular weight 5 MDa in lx phosphate buffered saline was prepared. Deposited 1-um microparticles were used to measure the thickness of murine colonic mucus hydrogel ex vivo, before and after exposure to this solution, as previously described. It is observed that the mucus hydrogel thickness reduces from an initial thickness of 66 microns to 25 microns, corresponding to 62% compression of the hydrogel. The osmotic pressure of this polymeric composition solution is expected to be approximately 2 kPa, less than the 0.7 MPa threshold that will induce osmotic diarrhea or osmotic stress in a host.

### Example 16

### Flory-Huggins Model Under Physiological Constraints

The control of the hydrogel volume by polymeric composition can be described according to the Flory-Huggins model of polymer solutions when under physiological constraints. In such cases, compression is defined as a reduction in the hydrogel volume to a volume less than or equal to 90% of its original volume, under the constraint that the polymer solution is chosen such that when it contacts the hydrogel at the target site or in transit through the host to the target site, is isotonic to or hypertonic to blood plasma, i.e. less than 0.74 MPa.

The calculations based on the Flory-Huggins model show that in some cases, polymers composed in a polymeric composition with a molecular weight of 400 Da, 6 kDa, and 200 kDa have a number of Kuhn segment of 1, 4, and 146, respectively. A compression of a hydrogel can be obtained by providing the polymeric composition comprising such polymers at a concentration of 10-20% w/v for y=4 and at a concentration of 0.05-20% w/v for y=146 when the other parameters in Eq. 6-7 are set as follows: Nₘ=1000, Xₛₘ=0, v⁰ₘ=0.13, v^{s}ₘ=0.01, Xₛₚ=0.45, and Xₘₚ=0.3 or Nₘ=628, Xₛₘ=0, v⁰ₘ=0.07, v^{s}ₘ=0.01, Xₛₚ=0.45, and Xₘₚ=0.3 or Nₘ=2026, Xₛₘ=0, v⁰ₘ=0.35, v^{s}ₘ=0.01, Xₛₚ=0.45, and Xₘₚ=0.3 or Nₘ=715, Xₛₘ=−0.2, v⁰ₘ=0.13, v^{s}ₘ=0.01, Xₛₚ=0.45, and Xₘₚ=0.3 or Nₘ=1247, Xₛₘ=0.1, v⁰ₘ=0.13, v^{s}ₘ=0.01, Xₛₚ=0.45, and Xₘₚ=0.3 or Nₘ=833, Xₛₘ=−0.1, v⁰ₘ=0.13, v^{s}ₘ=0.01, Xₛₚ=0.45, Xₘₚ=0.3. A compression was not obtained for y=1 at any tested concentration.

In some cases, a compression of a hydrogel can be obtained by providing the polymeric composition comprising such polymers at a concentration of 0.05-20% w/v for y=146 when the parameters in Eqs. 6-7 are set as follows: Nₘ=9425, Xₛₘ=0.45, v⁰ₘ=0.13, v^{s}ₘ=0.01, Xₛₚ=0.45 and Xₘₚ=0.3 or Nₘ=1000, Xₛₘ=0, v⁰ₘ=0.13, v^{s}ₘ=0.01, Xₛₚ=0.45 and Xₘₚ=0. A compression was not obtained for y=1 or y=4 at any tested concentration.

In some cases, a compression of a hydrogel can be obtained by providing the polymeric composition comprising such polymers at a concentration of 5-20% w/v for y=4 and at a concentration of 0.05-20% w/v for y=146 when the other parameters in Eq. 6-7 are set as follows: Nₘ=1000, Xₛₘ=0, v⁰ₘ=0.13, v^{s}ₘ=0.01, Xₛₚ=0.45 and Xₘₚ=0.5 or Nₘ=1000, Xₛₘ=0, v⁰ₘ=0.13, v^{s}ₘ=0.01, Xₛₚ=0.45 and Xₘₚ=0.4. A compression was not obtained for y=1 at any tested concentration.

In some cases, a compression of a hydrogel can be obtained by providing the polymeric composition comprising such polymers at a concentration of 15-20% w/v for y=4 and at a concentration of 0.05-20% w/v for y=146 when the other parameters in Eq. 6-7 are set as follows: Nₘ=1000, Xₛₘ=0, v⁰ₘ=0.13, v^{s}ₘ=0.01, Xₛₚ=0.45 and Xₘₚ=0.2. A compression was not obtained for y=1 at any tested concentration.

In some cases, polymers composed in a polymeric composition with a molecular weight of 400 Da, 6 kDa, and 200 kDa have a number of Kuhn segment of 1, 2, and 76, respectively. A compression of a hydrogel can be obtained by providing the polymeric composition comprising such polymers at a concentration of 0.05-20% w/v for y=76 when the other parameters in Eq. 6-7 are set as follows: Nₘ=1000, Xₛₘ=0, v⁰ₘ=0.13, v^{s}ₘ=0.01, Xₛₚ=0.45 and Xₘₚ=0.3. A compression was not obtained for y=1 and y=2.

In some cases, polymers composed in a polymeric composition with a molecular weight of 400 Da, 6 kDa, and 200 kDa have a number of Kuhn segment of 1, 11, and 611, respectively. A compression of a hydrogel can be obtained by providing the polymeric composition comprising such polymers at a concentration of 2-20% w/v for y=11 at a concentration of 0.05-20% w/v for y=611 when the other parameters in Eq. 6-7 are set as follows: Nₘ=1000, Xₛₘ=0, v⁰ₘ=0.13, v^{s}ₘ=0.01, Xₛₚ=0.45 and Xₘₚ=0.3. A compression was not obtained for y=1.

In summary, the polymeric composition and related methods and systems regulating the structure of biological hydrogels, are based on the features of biological hydrogel as a responsive biomaterial and reveal a mechanism of biological hydrogel reconstruction that can be integrated into the design and investigation involving therapeutic polymers, dietary fibers, and fiber-degrading gut microbes.

The examples set forth above are provided to give those of ordinary skill in the art a complete disclosure and description of how to make and use the embodiments of the materials, compositions, systems and methods of the disclosure, and are not intended to limit the scope of what the inventors regard as their disclosure. Those skilled in the art will recognize how to adapt the features of the exemplified methods to additional polymeric compositions and hydrogels in according to various embodiments and scope of the claims.

All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

In particular, it will be understood that various modifications may be made without departing from the spirit and scope of the present disclosure. Accordingly, other embodiments are within the scope of the following claims.

### REFERENCES

1. Cone R A (2009) Barrier properties of mucus. Adv Drug Deliv Rev 61(2):75-85.
2. Atuma C, Strugala V, Allen A, Holm L (2001) The adherent gastrointestinal mucus gel layer: thickness and physical state in vivo. Am J Physiol Liver Physiol 280(5):G922-G929.

3. Johansson M E V, et al. (2008) The inner of the two Muc2 mucin-dependent mucus layers in colon is devoid of bacteria. Proc Nalt Acad Sci USA 105(39):15064-15069.
4. Johansson M E V, Larsson J M H, Hansson G C (2011) The two mucus layers of colon are organized by the MUC2 mucin, whereas the outer layer is a legislator of host-microbial interactions. Proc Natl Acad Sci USA 108:4659-4665.
5. khanvilkar K (2001) Drug transfer through mucus. Adv Drug Deliv Rev 48(2-3):173-193.
6. Macfarlane S, Macfarlane G T, Cummings J H (2006) Review article: prebiotics in the gastrointestinal tract. Aliment Pharmacol Ther 24(5):701-714.
7. Hooper L V, Midtvedt T, Gordon J I (2002) How host-microbial interactions shape the nutrient environment of the mammalian intestine. Annu Rev Nutr 22:283-307.
8. DiPalma J A, DeRidder P H, Orlando R C, Kolts B E, Cleveland M V (2000) A randomized, placebo-controlled, multicenter study of the safety and efficacy of a new polyethylene glycol laxative. Am J Gastroenterol 95(2):446-450.
9. Dipalma J A, Brady C E (1989) Colon cleansing for diagnostic and surgical procedures: polyethylene glycol-electrolyte lavage solution. Am J Gastroenterol 84(9):1008-1016.
10. Barcelo A, et al. (2000) Mucin secretion is modulated by luminal factors in the isolated vascularly perfused rat colon. Gut 46(2):218-224.
11. Brownlee I A, Havler M E, Dettmar P W, Adrian A, Pearson J P (2003) Colonic mucus: secretion and turnover in relation to dietary fibre intake. Proc Nutr Soc 62(1):245-249.
12. Shimotoyodome A, Meguro S, Hase T, Tokimitsu I, Sakata T (2001) Sulfated polysaccharides, but not cellulose, increase colonic mucus in rats with loperamide-induced constipation. Dig Dis Sci 46(7):1482-1489.
13. Lidell M E, Moncada D M, Chadee K, Hansson G C (2006) Entamoeba histolytica cysteine proteases cleave the MUC2 mucin in its C-terminal domain and dissolve the protective colonic mucus gel. Proc Natl Acad Sci USA 103(24):9298-303.
14. Johansson M E V, et al. (2014) Bacteria penetrate the normally impenetrable inner colon mucus layer in both murine colitis models and patients with ulcerative colitis. Gut 63(2):281-291.
15. Bergstrom K S B, et al. (2010) Muc2 protects against lethal infectious colitis by disassociating pathogenic and commensal bacteria from the colonic mucosa. PLoS Pathog 6(5). doi:10.1371/j ournal.ppat.1000902.
16. Olmsted S S, et al. (2001) Diffusion of macromolecules and virus-like particles in human cervical mucus. Biophys J81(4):1930-1937.
17. Lai S K, Wang Y Y, Cone R, Wirtz D, Hanes J (2009) Altering mucus rheology to “solidify” human mucus at the nanoscale. PLoS One 4(1):6.
18. Johansson M E V, et al. (2010) Bacteria penetrate the inner mucus layer before inflammation in the dextran sulfate colitis model. PLoS One 5(8). doi:10.1371/journal.pone.0012238.
19. Johansson M E V, et al. (2015) Normalization of Host Intestinal Mucus Layers Requires Long-Term Microbial Colonization. Cell Host Microbe:1-11.
20. Valentine M T, et al. (2004) Colloid surface chemistry critically affects multiple particle tracking measurements of biomaterials. Biophys J86(6):4004-4014.
21. Jakobsson H E, et al. (2015) The composition of the gut microbiota shapes the colon mucus barrier. EMBO Rep 16(2):164-177.
22. Gustafsson J K, et al. (2012) An ex vivo method for studying mucus formation, properties, and thickness in human colonic biopsies and mouse small and large intestinal explants. Am J Physiol Gastrointest Liver Physiol 302(4):G430-8.
23. Matsui H, et al. (2006) A physical linkage between cystic fibrosis airway surface dehydration and Pseudomonas aeruginosa biofilms. Proc Natl Acad Sci USA 103(48):18131-18136.
24. Lai S K, et al. (2007) Rapid transport of large polymeric nanoparticles in fresh undiluted human mucus. Proc Natl Acad Sci USA 104(5):1482-1487.
25. Boyer R F (1945) Deswelling of gels by high polymer solutions. J Chem Phys 13(9):363-372.
26. Button B, et al. (2012) A periciliary brush promotes the lung health by separating the mucus layer from airway epithelia. Science 337(6097):937-941.
27. Brochard F (1981) Polymer networks swollen by a homopolymer solution. J Phys 42(3):505-511.
28. Vasilevskaya V V, Khokhlov A R (1992) Swelling and collapse of polymer gel in polymer solutions and melts. Macromolecules 25(1):384-390.
29. kayaman N, Okay O, Baysal B (1997) Phase transition of polyacrylamide gels in PEG solutions. Polym Gels Networks 5:167-184.
30. Saunders B R, Crowther H M, Vincent B (1997) Poly[(methyl methacrylate)-co-(methacrylic acid)] microgel particles: swelling control using pH, cononsolvency, and osmotic deswelling. Macromolecules 30(3):482-487.
31. Li H, et al. (2015) The outer mucus layer hosts a distinct intestinal microbial niche. Nat Commun 6:8292.
32. kobayashi K, Ogata H, Morikawa M, Iijima S, Harada N, Yoshida T, Brown W R, Inoue N, Hamada Y, Ishii H, Watanabe M H T (2002) Distribution and partial characterisation of IgG Fc. Gut 51:169-177.
33. Zindl C L, et al. (2013) IL-22-producing neutrophils contribute to antimicrobial defense and restitution of colonic epithelial integrity during colitis. Proc Natl Acad Sci USA 110(31):12768-73.
34. Heazlewood C K, et al. (2008) Aberrant mucin assembly in mice causes endoplasmic reticulum stress and spontaneous inflammation resembling ulcerative colitis. PLoS Med 5(3):0440-0460.
35. Engevik M a., et al. (2015) Human Clostridium difficile infection: altered mucus production and composition. Am J Physiol—Gastrointest Liver Physiol 308(6):G510-G524.
36. Ambort D, et al. (2012) Calcium and pH-dependent packing and release of the gel-forming MUC2 mucin. Proc Natl Acad Sci 109(15):5645-5650.
37. Flory P J (1953) Principles of Polymer Chemistry (Cornell University Press, Ithaca, N.Y.).
38. Rubinstein M, Colby R H (2003) Polymer Physics (OUP Oxford, New York).
39. Wang Y-Y, et al. (2008) Addressing the PEG Mucoadhesivity Paradox to Engineer Nanoparticles that “Slip” through the Human Mucus Barrier. Angew Chemie-International Ed 47(50):9726-9729.
40. Lotfy S (2009) Controlling degradation of low-molecular-weight natural polymer “dextrin” using gamma irradiation. Int J Biol Macromol 44(1):57-63.
41. White D R, Hudson P, Adamson J T (2003) Dextrin characterization by high-performance anion-exchange chromatography--pulsed amperometric detection and size-exclusion chromatography—multi-angle light scattering—refractive index detection. J Chromatogr A 997(1-2):79-85.
42. Pal S, Nasim T, Patra A, Ghosh S, Panda A B (2010) Microwave assisted synthesis of polyacrylamide grafted dextrin (Dxt-g-PAM): Development and application of a novel polymeric flocculant. Int J Biol Macromol 47(5):623-631.
43. Sun J, Zhao R, Zeng J, Li G, Li X (2010) Characterization of destrins with different dextrose equivalents. Molecules 15(8):5162-5173.
44. kim J H, Kim M R, Lee J H, Lee J W, Kim S K (2000) Production of high molecular weight pullulan by Aureobasidium pullulans using glucosamine. Biotechnol Lett 22(12):987-990.
45. Buliga G S, Brant D A (1987) Temperature and molecular weight dependence of the unperturbed dimensions of aqueous pullulan. Int J Biol Macromol 9(2):71-76.
46. kato T, Katsuki T, Takahashi A (1984) Static and dynamic solution properties of pullulan in a dilute solution. Macromolecules 17(1):1726-1730.
47. Lazaridou A, Roukas T, Biliaderis C G, Vaikousi H (2002) Characterization of pullulan produced from beet molasses by Aureobasidium pullulans in a stirred tank reactor under varying agitation. Enzyme Microb Technol 31(1-2):122-132.
48. Wiley B J, et al. (1993) Control of molecular weight distribution of the biopolymer pullulan produced by Aureobasidium pullulans. J Environ Polym Degrad 1(1):3-9.
49. Stephen A M, Phillips G O (2006) Food Polysaccharides and their Applications (CRC Press, Boca Raton).
50. Mestecky J, et al. (2005) Mucosal Immunology (Elsevier Science, San Diego).
51. Verdugo P (2012) Supramolecular dynamics of mucus. Cold Spring Harb Perspect Med 2(11). doi:10.1101/cshperspect.a009597.
52. Ambort D, Johansson M E V, Gustafsson J K, Ermund A, Hansson G C (2012) Perspectives on mucus properties and formation- lessons from the biochemical world. Cold Spring Harb Perspect Med 2(11). doi:10.1101/cshperspect.a014159.
53. Rubinstein M, Colby R H, Dobrynin A V. (1994) Dynamics of semidilute polyelectrolyte solutions. Phys Rev Lett 73(20):2776-2779.
54. Li H (2010) Smart Hydrogel Modelling (Springer-Verlag Berlin Heidelberg, New York).
55. Sing C E, Zwanikken J W, de la Cruz M O (2013) Effect of ion-ion correlations on polyelectrolyte gel collapse and reentrant swelling. Macromolecules 46(12):5053-5065.
56. Philippova O E, Rumyantsev A M, Kramarenko E Y, Khokhlov A R (2013) New type of swelling behavior upon gel ionization: Theory vs experiment. Macromolecules 46(23):9359-9367.
57. Dobrynin A V., Rubinstein M (2005) Theory of polyelectrolytes in solutions and at surfaces. Prog Polym Sci 30(11):1049-1118.
58. Perez-Vilar J (2007) Mucin granule intraluminal organization. Am J Respir Cell Mol Biol 36(2):183-190.
59. Lai S K, Wang Y-Y, Wirtz D, Hanes J (2009) Micro- and macrorheology of mucus. Adv Drug Deliv Rev 61(2):86-100.
60. Georgiades P, Pudney P D, Thornton D J, Waigh T A (2014) Particle tracking microrheology of purified gastrointestinal mucins. Biopolymers 101(4):366-377.
61. Cai L-H (2012) Structure and Function of Airway Surface Layer of the Human Lungs and Mobility of Probe Particles in Complex Fluids. Dissertation (University of North Carolina, Chapel Hill).
62. Szycher M (1991) High Performance Biomaterials: A Complete Guide to Medical and Pharmaceutical Applications (Taylor & Francis, Lancaster).
63. Lentle R G, Janssen P W M (2011) Mucins. The Physical Processes of Digestion (Springer Science & Buisness Media, 2011).
64. Sobota J A, Ferraro F, Back N, Eipper B A, Mains R E (2006) Not all secretory granules are created equal: partitioning of soluble content proteins. Mol Biol Cell 17:5038-5052.
65. Quillin M L, Matthews B W (2000) Accurate calculation of the density of proteins. Acta Crystallogr Sect D Biol Crystallogr 56(7):791-794.
66. Shogren R, Gerken T A, Jentoft N (1989) Role of glycosylation on the conformation and chain dimensions of O-linked glycoproteins: light-scattering studies of ovine submaxillary mucin. Biochemistry 28(13):5525-5536.
67. Aksoy N, Sheehan J (2002) Determination of polydispersity of a human colonic mucus glycoprotein using rate-zonal centrifugation and laser light-scattering. Turk J Med Sci 32:373-378.
68. Mikkelsen A, Stokke B T, Christensen B E, Elgsaeter A (1985) Flexibility and length of human bronchial mucin studied using low-shear viscometry, birefringence relaxation analysis, and electron microscopy. Biopolymers 24(9):1683-1704.
69. Jentoft N (1990) Why are proteins O-glycosylated? Trends Biochem Sci 15(8):291-294.
70. Bansil R, Stanley H E, Lamont J (1995) Mucin biophysics. Annu Rev Physiol 57:635-657.
71. Sheehan J K, et al. (2000) Physical characterization of the MUCSAC mucin: a highly oligomeric glycoprotein whether isolated from cell culture or in vivo from respiratory mucous secretions. Biochem J347:37-44.
72. Prasitnok K, Wilson MR (2013) A coarse-grained model for polyethylene glycol in bulk water and at a water/air interface. Phys Chem Chem Phys 15(40):17093-17104.
73. Fischer J, Paschek D, Geiger A, Sadowski G (2008) Modeling of aqueous poly (oxyethylene) solutions . 2 . Mesoscale simulations. J Phys Chem B:13561-13571.
74. kawaguchi S, et al. (1997) Aqueous solution properties of oligo- and poly(ethylene oxide) by static light scattering and intrinsic viscosity. Polymer (Guildf) 38(12):2885-2891.
75. Selser C, et al. (1991) Asymptotic behavior and long-range interactions in aqueous solutions of poly(ethylene oxide). Macromolecules 24:5943-5947.
76. Ziebacz N, Wieczorek SA, Kalwarczyk T, Fialkowski M, Holyst R (2011) Crossover regime for the diffusion of nanoparticles in polyethylene glycol solutions: influence of the depletion layer. Soft Matter 7(16):7181.
77. kienberger F, et al. (2000) Static and dynamical properties of single poly (ethylene glycol) molecules investigated by force spectroscopy. Single Mol 1(2):123-128.
78. Poon W C K, Andelman D (2006) Soft Condensed Matter Physics in Molecular and Cell Biology (CRC Press, Boca Raton).
79. Waters D J, et al. (2010) Morphology of photopolymerized end-linked poly (ethylene glycol) hydrogels by small-angle X-ray scattering. Macromolecules 43(16):6861-6870.
80. Dondi F, Guiochon G (2012) Theoretical Advancement in Chromatography and Related Separation Techniques (Springer Netherlands, Ferrara).
81. Tanford C (1961) Physical Chemistry of Macromolecules (Wiley, New York).
82. Mijnlieff P F, Wiegel F W (1978) Intrinsic viscosity and friction coefficient of polymer molecules in solution: Porous sphere model. J Polym Sci Polym Phys Ed 16(2):245-263.
83. Brandup J, Immergut E (1975) Polymer Handbook (John Wiley). 2nd Ed.
84. Momii T, Nose T (1989) Concentration-dependent collapse of polymer gels in solution of incompatible polymers. Macromolecules 22(3):1384-1389.
85. Ishidao T, Akagi M, Sugimoto H, Iwai Y, Arai Y (1993) Swelling behaviors of poly(N-isopropylacrylamide) gel in poly(ethylene glycol)-water mixtures. Macromolecules 26(26):7361-7362.
86. Ishidao T, et al. (1995) Swelling equilibria of poly(N-isopropylacrylamide) gel in glucose and starch aqueous solution. Fluid Phase Equilib 104:119-129.
87. Ito T, Yamazaki M, Ohnishi S (1989) Poly(ethylene glycol)-induced shrinkage of Sephadex gel. A model system for quantitative analysis of osmoelastic coupling. Biophys J 56(4):707-711.
88. Kiefer J, Naser M, Kamel A, Carnali J (1993) Osmotic deswelling of microgels by linear polyelectrolytes. Colloid Polym Sci 271(3):253-261.
89. Adachi H, Nishi S, Kotaka T (1982) Structure and Mechanical Properties of Sequential Interpenetrating Polymer Networks II. Complex-Forming Polyoxyethylene: Poly(acrylic acid) Systems. Polym J 14(12):985-992.
90. Nishi S, Kotaka T (1985) Complex-Forming Poly(oxyehtylene):Poly(acrylic acid) Interpenetrating Polymer Networks. 1. Preparation, Structure and Viscoelastic Properties. Macromolecules 18(8):1519-1525.
91. Chen H-L, Morawetz H (1982) Kinetics of Polymer Complex Interchange in Poly(acrylic acid)-Poly(oxyethylene) Solutions. Macromolecules 15:1445-1447.
92. Bednar B, Morawetz H, Shafer J (1984) Kinetics of the Cooperative Complex Formation and Dissociation of Poly(acrylic acid) and Poly(oxyethylene). Macromolecules 17:1634-1636.
93. Philippova O E, Karibyants N S, Starodubtzev S G (1994) Conformational Changes of Hydrogels of Poly(methacrylic acid) Induced by Interaction with Poly(ethylene glycol). Macromolecules 27(9):2398-2401.
94. Sonnenburg J L (2005) Glycan foraging in vivo by an intestine-adapted bacterial symbiont. Science 307(5717):1955-1959.
95. Loesche W J (1969) Effect of bacterial contamination on cecal size and cecal contents of gnotobiotic rodents. J Bacteriol 99(2):520-526.
96. Hoverstad T, Midtvedt T (1986) Short-chain fatty acids in germfree mice and rats. J Nutr 116(9):1772-1776.
97. Heneghan J (2012) Germfree Research: Biological Effect of Gnotobiotic Environments (Elsevier Science, New York).
98. Spiro R G (1970) Glycoproteins. Annu Rev Biochem 39(1):599-638.
99. Stepto, R. F., Dispersity in polymer science (IUPAC Recommendations 2009). Pure and Applied Chemistry, 2009. 81(2): p. 351-353.
100. Lai, S. K., et al., Nanoparticles reveal that human cervicovaginal mucus is riddled with pores larger than viruses. Proceedings of the National Academy of Sciences, 2010. 107(2): p. 598-603.
101. Lai, S. K., et al., Altering mucus rheology to “solidify” human mucus at the nanoscale. Plos One, 2009. 4(1): p. 1-6.
102. Gipson, I. K., et al., The Amount of MUCSB Mucin in Cervical Mucus Peaks at Midcycle 1. The Journal of Clinical Endocrinology & Metabolism, 2001. 86(2): p. 594-600.
103. Button, B., et al., A periciliary brush promotes the lung health by separating the mucus layer from airway epithelia. Science, 2012. 337(6097): p. 937-941.
104. kvietys, Peter R., Ahmed Yaqinuddin, and Wael Al Kattan. “Gastrointestinal Mucosal Defense System.” Colloquium Series on Integrated Systems Physiology: From Molecule to Function to Disease. Vol. 6. No. 5. Morgan & Claypool Life Sciences, 2014.
105. Bansil, R., et al., The influence of mucus microstructure and rheology in Helicobacter pylori infection. Frontiers in Immunology, 2013. 4(October): p. 1-12.
106. Lai, S. K., et al., Micro- and macrorheology of mucus. Advanced Drug Delivery Reviews, 2009. 61(2): p. 86-100.
107. Yeh, Yu Jie, Lydia Yu Li Law, and Chin Leong Lim. “Gastrointestinal response and endotoxemia during intense exercise in hot and cool environments.” European journal of applied physiology 113.6 (2013): 1575-1583.
108. Lim, Chin Leong. “Unusually low plasma concentration of lipopolysaccharide following 160-km race.” Brain, behavior, and immunity 21.4 (2007): 514.
109. Nieman, David C., et al. “Ibuprofen use, endotoxemia, inflammation, and plasma cytokines during ultramarathon competition.” Brain, behavior, and immunity 20.6 (2006): 578-584.
110. Makino, S., et al. “Immunomodulatory effects of polysaccharides produced by Lactobacillus delbrueckii ssp. bulgaricus OLL1073R-1.” Journal of dairy science 89.8 (2006): 2873-2881.
111. Arihara, Keizo, Takahiro Toba, and Susumu Adachi. “Immunofluorescence microscopic studies on distribution of Lactobacillus kefiranofaciens and Lactobacillus kefir in kefir grains.” International journal of food microbiology 11.2 (1990): 127-134.
112. Yuan, Y., et al. “Optimum duration of regimens for Helicobacter pylori eradication (Review).” (2013).
113. Hampson, F. C., et al. “Alginate rafts and their characterisation.” International journal of pharmaceutics 294.1 (2005): 137-147.
114. Donnarumma, Giovanna, et al. “Lactobacillus crispatus L1: high cell density cultivation and exopolysaccharide structure characterization to highlight potentially beneficial effects against vaginal pathogens.” BMC microbiology 14.1 (2014): 137.
115. Brandrup, J., et al., Polymer handbook. Vol. 1999. 1999: Wiley New York.

1. A method to control an overall volume of a biological hydrogel, the method comprising
contacting the biological hydrogel with one or more polymers of a molecular weight from 100 Da to 5 MDa at a concentration from 0.05-80% w/v, the molecular weight and the concentration of the one or more polymers selected to obtain a change in the overall volume of the biological hydrogel according to a Flory-Huggins model.

2. The method of claim 1, wherein the one or more polymer, the molecular weight and the concentration are selected by
numerically solving the Flory-Huggins model for one or more set overall volumes of the biological hydrogel and the one or more polymers;
providing a look-up table connecting the one or more set overall volumes of the biological hydrogel, with molecular weights and concentrations of the one or more polymers based on parameters of the Flory-Huggins model related to the one or more polymers and associated with the one or more set overall volumes of the biological hydrogel in the numerically solved Flory-Huggins model; and
selecting a specific combination of concentrations and molecular weights of the polymer corresponding to a specific set overall volume.

3. The method of claim 2, further comprising
identifying a percentage compression or decompression based on the numeric solution of the Flory-Huggins model;
providing a look-up table connecting the one or more set overall volumes of the biological hydrogel, with molecular weights and concentrations of the one or more polymers based on parameters of the Flory-Huggins model related to the one or more polymers and associated with the identified percentage compression or decompression of the biological hydrogel based on the numerically solved Flory-Huggins model; and
selecting a specific combination of concentrations and molecular weights of the polymer corresponding to a specific percent compression and/or percent decompression.

4. The method of claim 1, wherein the Flory Huggins model comprises the following equations
$$\begin{matrix}\frac{{μ}_{S}^{i n}}{RT} = \frac{1}{{N}_{M}} ( {v}_{M}^{1 / 3} {v}_{M}^{{0}^{2 / 3}} - \frac{{v}_{M}}{2} ) + ln {v}_{S}^{i n} + 1 - {v}_{S}^{i n} - \frac{{v}_{P}^{i n}}{y} + ( {χ}_{SM} {v}_{M} + {χ}_{SP} {v}_{P}^{i n} ) ( 1 - {v}_{S}^{i n} ) - {χ}_{MP} {v}_{M} {v}_{P}^{i n} & ( Eq . 1 ) \\ \frac{{μ}_{S}^{out}}{RT} = ln ( 1 - ϕ ) + ϕ ( 1 - \frac{1}{y} ) + {χ}_{SM} {ϕ}^{2} & ( Eq . 2 ) \\ \frac{{μ}_{P}^{i n}}{yRT} = \frac{1}{{N}_{M}} ( {v}_{M}^{1 / 3} {v}_{M}^{{0}^{2 / 3}} - \frac{{v}_{M}}{2} ) + \frac{1}{y} ln {v}_{P}^{i n} + \frac{1}{y} ( 1 - {v}_{P}^{i n} ) - {v}_{S}^{i n} + ( {χ}_{SP} {v}_{S}^{i n} + {χ}_{MP} {v}_{M} ) ( 1 - {v}_{P}^{i n} ) - {χ}_{SM} {v}_{S}^{i n} {v}_{M} & ( Eq . 3 ) \\ \frac{{μ}_{P}^{out}}{yRT} = \frac{1}{y} ln ϕ - 1 + ϕ + \frac{1}{y} ( 1 - ϕ ) + {{χ}_{SM} ( 1 - ϕ )}^{2} & ( Eq . 4 )\end{matrix}$$

5. The method of claim 4, wherein the Flory Huggins model comprises the following equations
$$\begin{matrix} \frac{1}{{N}_{M}} ( {v}_{M}^{{S}^{1 / 3}} {v}_{M}^{{0}^{2 / 3}} - \frac{{v}_{M}^{S}}{2} ) + ln ( 1 - {v}_{M}^{S} ) + {v}_{M}^{S} + {χ}_{SM} {v}_{M}^{{S}^{2}} = 0 & ( Eq . 5 ) \\ \frac{1}{{N}_{M}} ( {v}_{M}^{1 / 3} {v}_{M}^{{0}^{2 / 3}} - \frac{{v}_{M}}{2} ) + ln ( 1 - {v}_{M} - {v}_{P}^{i n} ) + {v}_{M} + {v}_{P}^{i n} - \frac{{v}_{P}^{i n}}{y} + ( {χ}_{SM} {v}_{M} + {χ}_{SP} {v}_{P}^{i n} ) ( {v}_{M} + {v}_{P}^{i n} ) - {χ}_{MP} {v}_{M} {v}_{P}^{i n} = ln ( 1 - ϕ ) + ϕ ( 1 - \frac{1}{y} ) + {χ}_{SM} {ϕ}^{2} & ( Eq . 6 ) \\ \frac{1}{{N}_{M}} ( {v}_{M}^{1 / 3} {v}_{M}^{{0}^{2 / 3}} - \frac{{v}_{M}}{2} ) + \frac{1}{y} ln {v}_{P}^{i n} + \frac{1}{y} ( 1 - {v}_{P}^{i n} ) - ( 1 - {v}_{M} - {v}_{P}^{i n} ) + ( {χ}_{SP} \cdot ( 1 - {v}_{M} - {v}_{P}^{i n} ) + {χ}_{MP} {v}_{M} ) ( 1 - {v}_{P}^{i n} ) - {χ}_{SM} ( 1 - {v}_{M} - {v}_{P}^{i n} ) {v}_{M} = \frac{1}{y} ln ϕ - 1 + ϕ + \frac{1}{y} ( 1 - ϕ ) + {{χ}_{SM} ( 1 - ϕ )}^{2} & ( Eq . 7 )\end{matrix}$$

6. The method of claim 1 wherein the Flory Huggins model further comprises the following equation
Compression %=100%×(1−v_{M}^{s}/v_{M})

7. The method of claim 1, wherein the one or more polymers have a molecular weight from 100 Da to 5 MDa at a concentration from 0.05-20% w/v.

8. The method of claim 1, wherein the one or more polymers have a molecular weight from 100 Da to 5 MDa at a concentration from 30-70% w/v.

9. The method of claim 1, wherein the one or more polymers have a molecular weight from 100 Da to 5 MDa at a concentration from 65-70% w/v.

10. The method of claim 1, wherein the one or more polymers have a molecular weight of about 200 kDa at a concentration from 0.05-20% w/v.

11. The method of claim 1, wherein the one or more polymers have a molecular weight 6 kDa at a concentration from 30-70% w/v.

12. The method of claim 1, wherein the one or more polymers have a molecular weight from 100 Da to 5 MDa at a concentration from 0.05-30% w/v.

13. A method of controlling an overall volume/thickness/mesh size of a biological hydrogel, the method comprising:
contacting the biological hydrogel with one or more polymers having a molecular weight from 100 Da to 5 MDa at a concentration from 0.05%-80% w/v, the molecular weight and the concentration selected to modify an osmotic pressure difference between an external osmotic pressure externally applied to an external surface of the biological hydrogel and an internal osmotic pressure internally applied to the external surface of the biological hydrogel.

14. The method of claim 13, wherein the molecular weight and the concentration of the one or more polymers are selected by
providing a look-up table connecting one or more concentrations of the one or more polymers with one or more corresponding external osmotic pressure, one or more corresponding internal osmotic pressure and/or one or more corresponding osmotic pressure difference for the biological hydrogel and
selecting from the look-up table the one or more concentrations of the one or more polymers associated with a desired external osmotic pressure, internal osmotic pressure and/or osmotic pressure difference.

15. The method of claim 13, wherein the osmotic pressure difference is provided by
detecting, for a given amount of the one or more polymers, a ratio between a concentration of the one or more polymers inside the biological hydrogel and a concentration of the one or more polymers outside the biological hydrogel ;
providing an internal osmotic pressure corresponding to the detected concentration of the one or more polymers inside the biological hydrogel and an external osmotic pressure corresponding to concentration of the one or more polymers outside the biological hydrogel; and
providing an osmotic pressure difference between the provided internal osmotic pressure and external osmotic pressure.

16. The method of claim 13, wherein the one or more polymers have a polymer size greater than a mesh size of the biological hydrogel.

17. The method of claim 13, wherein the one or more polymers have a molecular weight from 200 kDa to 5MDa and at a concentration from 0.05-20% w/v.

18. The method of claim 13, wherein the molecular weight and the concentration are selected to obtain a total osmotic pressure less than 0.74 MPa.

19. A method of compressing a biological hydrogel, comprising:
contacting the biological hydrogel with one or more polymers having a molecular weight from 100 Da to 5 MDa at a concentration from 0.05%-80% w/v,
the molecular weight and the concentration of the one or more polymers selected to obtain an osmotic pressure difference between an external osmotic pressure externally applied to an external surface of the biological hydrogel and an internal osmotic pressure internally applied to the external surface of the biological hydrogel,
the osmotic pressure difference equal to or greater than 10% of an elastic modulus of the biological hydrogel.

20. The method of claim 19, wherein the molecular weight and the concentration of the one or more polymers are selected to obtain a total osmotic pressure less than 0.74 MPa.

21. The method of claim 19, further comprising:
removing the polymeric composition from the hydrogel to decrease the osmotic pressure difference of the polymeric composition.

22. The method of claim 19, further comprising:
contacting microbes with the compressed biological hydrogel and/or the polymeric composition to obtain a decrease in the osmotic pressure difference of the polymeric composition, the microbes being capable of degrading the polymeric composition.

23. A method of compressing a colonic mucus hydrogel at a base state, comprising:
contacting the biological hydrogel with one or more polymers having a molecular weight from 100 Da to 5 MDa at a concentration from 0.05-80% w/v, the molecular weight and the concentration being selected to obtain an osmotic pressure difference equal to or greater than 10% of an elastic modulus of the biological hydrogel and a total osmotic pressure less than 0.74 MPa,
wherein the osmotic pressure difference is a difference between an external osmotic pressure externally applied to an external surface of the biological hydrogel and an internal osmotic pressure internally applied to the external surface of the biological hydrogel.

24. The method of claim 23, wherein the one or more polymers have a molecular weight from 200 kDa to 5MDa at a concentration from 0.05-2.0% w/v.

25. The method of claim 1, wherein the one or more polymers are amphiphilic.

26. The method of claim 1, wherein the one or more polymers comprises PEG having a molecular weight from 400Da to 200 kDa at a concentration from 2-80% w/v.

27. The method of claim 1, wherein the one or more polymers comprise a dietary fiber.

28. The method of claim 1, wherein the one or more polymers comprise a polysaccharide.

29. The method of claim 28, wherein the polysaccharide is selected from dextrin, pectin and pullulan.

30. The method of claim 1, wherein the biological hydrogel is a biological hydrogel selected from a group comprising mucus layer, extracellular matrix and biofilm extracellular polymeric substance.

31. The method of claim 30, wherein the mucus is selected from a group comprising colonic mucus, cervicovaginal mucus, airway mucus, and nasal mucus.

32. The method of claim 30, wherein the biological hydrogel has a mesh size from 100 to 250 nm.

33. The method of claim 1, wherein the contacting is performed in vivo by administrating the polymeric composition to an individual.

34. The method of claim 33, wherein the administrating is performed through administration routes selected from the group comprising oral ingestion, inhalation, intranasal, rectal and vaginal administration, topical application, intravenous injections and subcutaneous injections.

35. A polymeric composition to control a structure of a biological hydrogel, the polymeric composition comprising in a suitable vehicle, one or more polymers of a molecular weight from 100 Da to 5 MDa at a concentration from 0.05-80% w/v, the molecular weight and concentration of the one or more polymers selected to obtain a change in the overall volume, mesh size and/or thickness of the biological hydrogel according to the method of claim 1.

36. A system to control a structure of a biological hydrogel, the system comprising one or more polymeric compositions according to claim 35 and a look-up table connecting one or more molecular weight and/or one or more concentrations of one or more polymers in the polymeric composition with at least one of: one or more overall volumes, one or more mesh size and one or more thicknesses of the biological hydrogel.

37. A system to control a structure of a biological hydrogel, the system comprising one or more polymeric compositions according to claim 35 and a look-up table connecting one or more molecular weight and/or one or more concentrations of one or more polymers in the polymeric composition with a percent compression and/or a percent decompression of the biological hydrogel.

38. The system of claim 36, wherein when the biological hydrogel is a colonic mucus hydrogel, the system further comprises osmotic laxatives and/or antidiarrheal agents and/or antihelminthic drugs and/or antimicrobial drugs and/or anti-IBD drugs and/or anti-IBS drugs.

39. The system of claim 36, wherein when the biological hydrogel is a cervicovaginal or vaginal mucus hydrogel, the system further comprises personal lubricants and/or barrier contraceptives and/or spermicide compounds.

40. The system of claim 36, wherein when the biological hydrogel is a cervicovaginal or vaginal mucus hydrogel, the one or more polymeric compositions are provided by polymer-producing probiotics.

41. The system of claim 40, wherein the polymer-producing probiotics comprises Lactobacillus crispatus.
